(19) United States
(12) Patent Application Publication
(10) Pub. No.: US 2022/0093241 A1
(43) Pub. Date: Mar. 24, 2022

(54) CORRELATING INTERACTION EFFECTIVENESS TO CONTACT TIME USING SMART FLOOR TILES

(71) Applicant: SCANALYTICS, INC., Milwaukee, WI (US)

(72) Inventor: Joseph Scanlin, Milwaukee, WI (US)

(73) Assignee: SCANALYTICS, INC., Milwaukee, WI (US)

(21) Appl. No.: 17/544,752

(22) Filed: Dec. 7, 2021

Related U.S. Application Data
(63) Continuation-in-part of application No. 17/116,582, filed on Dec. 9, 2020.
Application No. 17/116,582 is a continuation-in-part of application No. 16/696,802, filed on Nov. 26, 2019, now Pat. No. 10,954,677.
(60) Provisional application No. 63/122,603, filed on Dec. 8, 2020.
(60) Provisional application No. 63/122,799, filed on Dec. 8, 2020.
(60) Provisional application No. 63/122,700, filed on Dec. 8, 2020.
(60) Provisional application No. 62/956,532, filed on Jan. 2, 2020.

(51) Int. Cl. G16H 40/20; E04F 15/10; G06V 20/40; G06V 20/52; G06N 20/00

(52) U.S. Cl. CPC G16H 40/20; E04F 15/105; G06N 20/00; G06V 20/46; G06V 20/52; E04F 15/107

(57) ABSTRACT

A method for correlating interaction effectiveness to contact time, the method including receiving first data pertaining to one or more first time and location events caused by a first object in a physical space, wherein the one or more first time and location events comprise one or more first times and one or more first locations of the first object in the physical space; receiving second data pertaining to one or more second time and location events caused by a second object in the physical space, wherein the one or more second time and location events comprise one or more second times and one or more second locations of the second object in the physical space; determining a interaction time between the first object and the second object; receiving interaction effectiveness data; and generating a time-effectiveness data point by associating the interaction effectiveness data with the interaction time.

### CROSS-REFERENCE TO RELATED APPLICATIONS

The present application claims priority to and the benefit of U.S. Provisional Patent Application No. 63/122,603, titled “CORRELATING INTERACTION EFFECTIVENESS TO CONTACT TIME USING SMART FLOR TILES” filed Dec. 8, 2020, and the present application is a continuation-in-part of U.S. Non-Provisional application Ser. No. 17/116,582, titled “PATH ANALYTICS OF PEOPLE IN A PHYSICAL SPACE USING SMART FLOOR TILES” filed Dec. 9, 2020, which claims priority to U.S. Provisional Application No. 62/956,532, titled “PREVENTION OF FALL EVENTS USING INTERVENTIONS BASED ON DATA ANALYTICS” filed Jan. 2, 2020, and which is a continuation-in-part of U.S. Non-Provisional application Ser. No. 16/696,802, titled “CONNECTED MOULDING FOR USE IN SMART BUILDING CONTROL” filed Nov. 26, 2019.

The present application claims priority to and the benefit of U.S. Provisional Patent Application No. 63/122,799, titled “ENVIRONMENT CONTROL USING MOULDING SECTIONS,” filed Dec. 8, 2020.

The present application claims priority to and the benefit of U.S. Provisional Patent Application No. 63/122,700, titled “SECURITY SYSTEM IMPLEMENTED IN A PHYSICAL SPACE USING SMART FLOOR TILES,” filed Dec. 8, 2020.

The contents of all of these applications are incorporated herein by reference in their entirety for all purposes.

### TECHNICAL FIELD

This disclosure relates to data analytics. More specifically, this disclosure relates to path analytics of people in a physical space using smart floor tiles.

### BACKGROUND

Practitioners, such as doctors, often have busy schedules and limited time available to talk with or treat patients. Pressure can exist to treat more patients, resulting in a lower time spent with each patient. However, it is understood that there is a benefit to doctors and other practitioners spending more time discussing health concerns with patients, discussing potential treatments with patients, and treating patients. After a certain point, however, the benefits of further interaction may be reduced substantially. Thus, there may be an ideal range of time for a practitioner to spend interacting with a patient to reach a maximum treatment effectiveness. This may change based on practitioners, medical conditions (physical or psychological) experienced, fields of medicine studied, or other relevant factors. Thus, it would be useful to have a way to correlate interaction time between patients and practitioners with treatment effectiveness.

Further, comfortable environments may include desired temperatures of a physical space for people to occupy. Different people may prefer different environments. Buildings may include conventional heating and cooling systems that attempt to provide a comfortable environment for people to occupy. Conventional heating and cooling systems may not control the environment of a physical space efficiently, accurately, and/or as desired by some people.

In addition, it may be desirable to track people as they move around certain physical spaces. For example, in a nursing home, a patient may have Alzheimer's disease or another neurodegenerative disease. Knowing the whereabouts of the patient may be important because the patient may forget where they are on their own as a symptom of their neurodegenerative disease. If the patient forgets where they are, and no one else knows where the patient is located, it may lead to an undesirable situation.

### SUMMARY

In one embodiment, a method for correlating interaction effectiveness to contact time is disclosed. The method includes receiving first data pertaining to one or more first time and location events caused by a first object in a first physical space, wherein the one or more first time and location events comprise one or more first times and one or more first locations of the first object in the first physical space; receiving second data pertaining to one or more second time and location events caused by a second object in the first physical space, wherein the one or more second time and location events comprise one or more second times and one or more second locations of the second object in the first physical space; based on the first data and the second data, determining a first interaction time between the first object and the second object; receiving first interaction effectiveness data pertaining to interaction effectiveness; and generating a first time-effectiveness data point by associating the first interaction effectiveness data with the first interaction time.

In one embodiment, a method for environment control using a moulding section is disclosed. The method includes receiving data from a sensor in the moulding section, determining, based on the data, whether a person is near the sensor, and determining an operating state of a device included in the moulding section. The device performs the environment control of a physical space in which the moulding section is located. Responsive to determining that the person is near the sensor and the operating state of the device, the method includes changing the device to operate in a second operating state to change a temperature of the physical space in which the moulding section is located.

In one embodiment, a method may include receiving data from a sensor in a smart floor tile, determining, based on the data, whether a person is present in a physical space including the smart floor tile, determining an operating state of a device included in a moulding section. The device performs environment control of the physical space in which the moulding section is located. Responsive to determining that the person is present in the physical space and the operating state of the device, the method may include changing the device to operate in a second operating state to change a temperature of the physical space.

In one embodiment, a method for performing an action based on a location of a person in a physical space is disclosed. The method includes receiving, from one or more smart floor tiles located in the physical space, data pertaining to the location of the person. The one or more smart floor tiles include one or more sensing devices capable of obtaining one or more pressure measurements, and the data includes the one or more pressure measurements. The method also includes determining, based on the data, a distance from the location of the person to a location of an object in the physical space, determining whether the distance from the location of the person to the location of the object satisfies a threshold distance, and responsive to determining the distances satisfies the threshold distance, transmitting, via a processing device, a control signal to a device to cause the device to perform an action. The device is distal from the processing device.

In one embodiment, a tangible, non-transitory computer-readable medium stores instructions that, when executed, cause a processing device to perform any operation of any method disclosed herein.

In one embodiment, a system includes a memory device storing instructions and a processing device communicatively coupled to the memory device. The processing device executes the instructions to perform any operation of any method disclosed herein.

Other technical features may be readily apparent to one skilled in the art from the following figures, descriptions, and claims.

### BRIEF DESCRIPTION OF THE DRAWINGS

For a detailed description of example embodiments, reference will now be made to the accompanying drawings in which:

FIGS. 1A-1E illustrate various example configurations of components of a system according to certain embodiments of this disclosure;

FIG. 2 illustrates an example component diagram of a moulding section according to certain embodiments of this disclosure;

FIG. 3 illustrates an example backside view of a moulding section according to certain embodiments of this disclosure;

FIG. 4 illustrates a network and processing context for smart building control according to certain embodiments of this disclosure;

FIG. 5 illustrates aspects of a smart floor tile according to certain embodiments of this disclosure;

FIG. 6 illustrates a master control device according to certain embodiments of this disclosure;

FIG. 7A illustrate an example of a method for generating a path of a person in a physical space using smart floor tiles according to certain embodiments of this disclosure;

FIG. 7B illustrates an example of a method continued from FIG. 7A according to certain embodiments of this disclosure;

FIG. 8 illustrates an example of a method for filtering paths of objects presented on a display screen according to certain embodiments of this disclosure;

FIG. 9 illustrates an example of a method for presenting a longest path of an object in a physical space according to certain embodiments of this disclosure;

FIG. 10 illustrates an example of a method for presenting amount of times objects spent at certain zones in a physical space according to certain embodiments of this disclosure;

FIG. 11 illustrates an example of a method for determining where to place objects based on paths of people according to certain embodiments of this disclosure;

FIG. 12 illustrates an example of a method for overlaying paths of objects based on criteria according to certain embodiments of this disclosure;

FIG. 13A illustrates an example user interface presenting paths of people in a physical space according to certain embodiments of this disclosure;

FIG. 13B illustrates an example user interface presenting a filtered path of a person in a physical space according to certain embodiments of this disclosure;

FIG. 13C illustrates an example user interface presenting information pertaining to paths of people in a physical space according to certain embodiments of this disclosure;

FIG. 13D illustrates an example user interface presenting other information pertaining to a path of a person in a physical space and a recommendation where to place an object in the physical space based on path analytics according to certain embodiments of this disclosure;

FIG. 14 illustrates an example computer system according to embodiments of this disclosure;

FIG. 15A illustrates an example of a method for generating a path of a person in a physical space using smart floor tiles according to certain embodiments of this disclosure;

FIG. 15B illustrates an example of a method continued from FIG. 15A according to certain embodiments of this disclosure;

FIG. 16A illustrates an example of a method for measuring correlations between treatment effectiveness and patient to practitioner contact time using smart floor tiles according to certain embodiments of this disclosure;

FIG. 16B illustrates an example of a method continued from FIG. 16A according to certain embodiments of this disclosure;

FIG. 17 illustrates an example of a physical space in which the method described in FIGS. 16A-16B can be applied according to certain embodiments of this disclosure;

FIG. 18 illustrates an example of a graphical user interface displaying a correlation between treatment effectiveness and patient to practitioner contact time according to certain embodiments of this disclosure;

FIGS. 100A-100E illustrate various example configurations of components of a system according to certain embodiments of this disclosure;

FIG. 200 illustrates an example component diagram of a moulding section according to certain embodiments of this disclosure;

FIG. 300 illustrates an example backside view of a moulding section according to certain embodiments of this disclosure;

FIG. 400 illustrates a network and processing context for smart building control using directional occupancy sensing and fall prediction/prevention 4

according to certain embodiments of this disclosure;

FIG. 500 illustrates aspects of a smart floor tile according to certain embodiments of this disclosure;

FIG. 600 illustrates a master control device according to certain embodiments of this disclosure;

FIG. 700A illustrate an example of a method for predicting a fall event according to certain embodiments of this disclosure;

FIG. 700B illustrates an example architecture including machine learning models to perform the method of FIG. 700A according to certain embodiments of this disclosure;

FIG. 800 illustrates example interventions according to certain embodiments of this disclosure;

FIG. 900 illustrates example parameters that may be monitored according to certain embodiments of this disclosure;

FIG. 1000 illustrates an example of a method for using gait baseline parameters to determine an amount of gait deterioration according to certain embodiments of this disclosure;

FIG. 1100 illustrates an example of a method for subtracting data associated with certain people from gait analysis according to certain embodiments of this disclosure;

FIG. 1200A-B illustrate an overhead view of an example for subtracting data associated with certain people from gait analysis according to certain embodiments of this disclosure;

FIG. 1300 illustrates an example of a method for controlling an environment using a moulding section based on data received from a sensor of the moulding section according to certain embodiments of this disclosure;

FIG. 1400 illustrates an example of a method for controlling an environment using a moulding section based on data received from a smart floor tile according to certain embodiments of this disclosure;

FIG. 1500 illustrates an example physical space having an environment controlled by a moulding section according to certain embodiments of this disclosure;

FIG. 1600 illustrates an example computer system according to embodiments of this disclosure.

FIGS. 2000A-2000E illustrate various example configurations of components of a system according to certain embodiments of this disclosure;

FIG. 3000 illustrates an example component diagram of a moulding section according to certain embodiments of this disclosure;

FIG. 4000 illustrates an example backside view of a moulding section according to certain embodiments of this disclosure;

FIG. 5000 illustrates a network and processing context for smart building control according to certain embodiments of this disclosure;

FIG. 6000 illustrates aspects of a smart floor tile according to certain embodiments of this disclosure;

FIG. 7000 illustrates a master control device according to certain embodiments of this disclosure;

FIG. 8000A illustrate an example of a method for generating a path of a person in a physical space using smart floor tiles according to certain embodiments of this disclosure;

FIG. 8000B illustrates an example of a method continued from FIG. 8000A according to certain embodiments of this disclosure;

FIG. 9000 illustrates an example of a method for filtering paths of objects presented on a display screen according to certain embodiments of this disclosure;

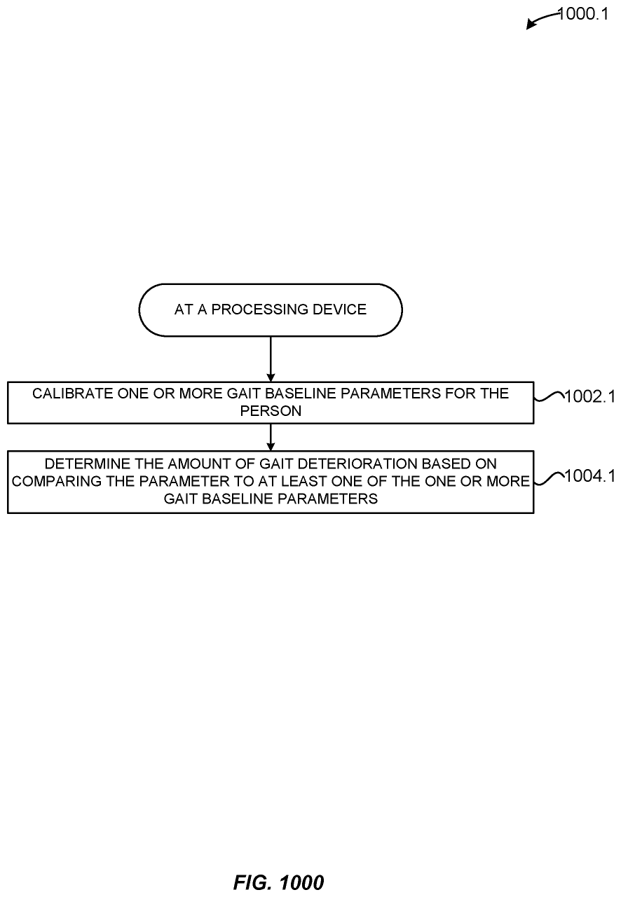

FIG. 10000 illustrates an example of a method for presenting a longest path of an object in a physical space according to certain embodiments of this disclosure;

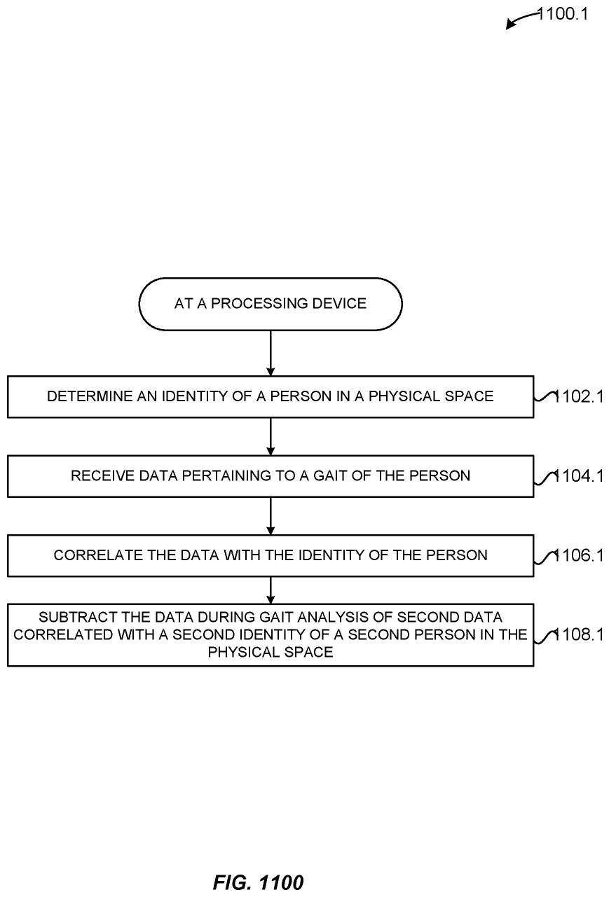

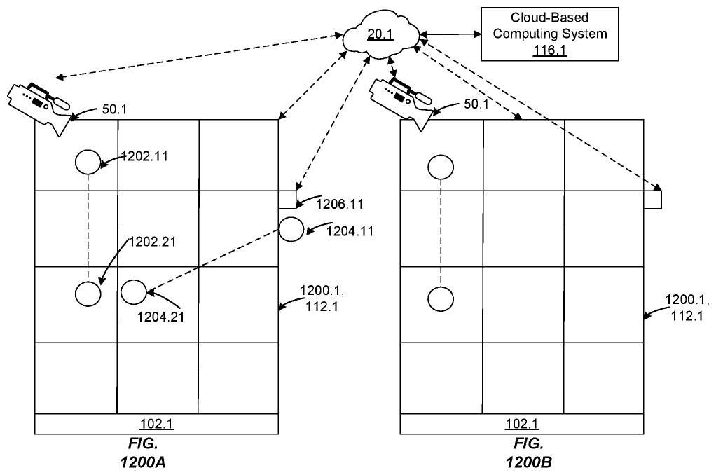

FIG. 11000 illustrates an example of a method for presenting amount of times objects spent at certain zones in a physical space according to certain embodiments of this disclosure;

FIG. 12000 illustrates an example of a method for determining where to place objects based on paths of people according to certain embodiments of this disclosure;

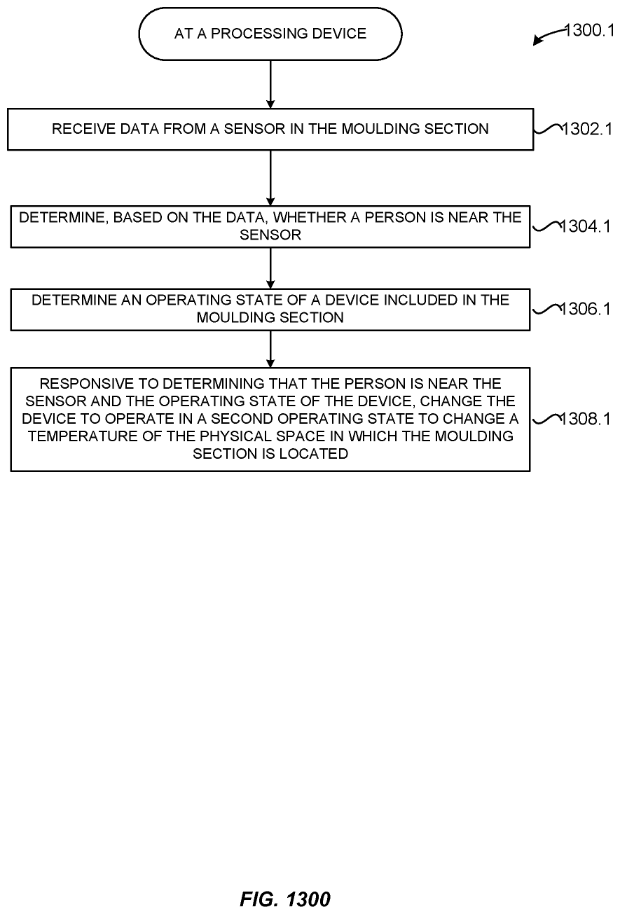

FIG. 13000 illustrates an example of a method for overlaying paths of objects based on criteria according to certain embodiments of this disclosure;

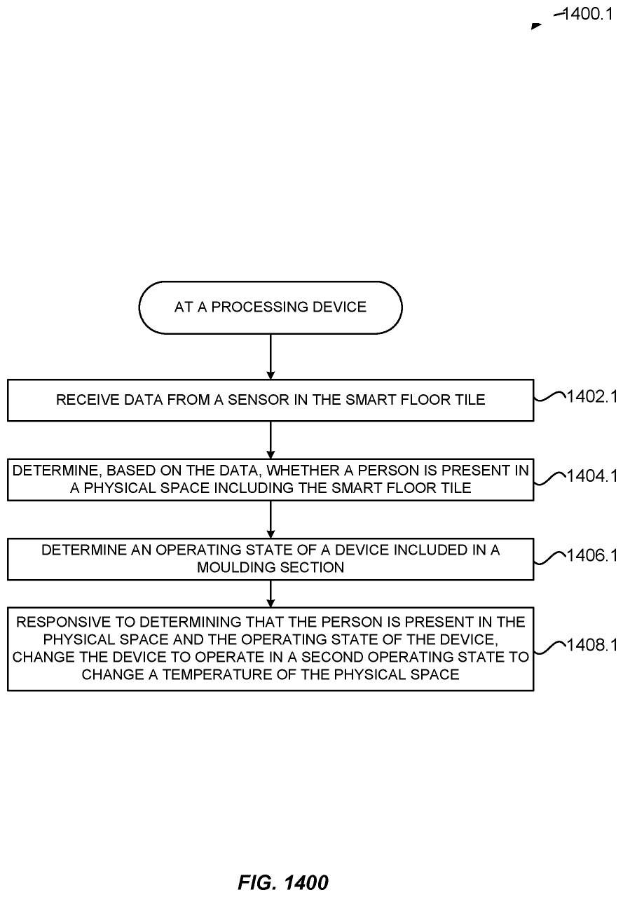

FIG. 14000A illustrates an example user interface presenting paths of people in a physical space according to certain embodiments of this disclosure;

FIG. 14000B illustrates an example user interface presenting a filtered path of a person in a physical space according to certain embodiments of this disclosure;

FIG. 14000C illustrates an example user interface presenting information pertaining to paths of people in a physical space according to certain embodiments of this disclosure;

FIG. 14000D illustrates an example user interface presenting other information pertaining to a path of a person in a physical space and a recommendation where to place an object in the physical space based on path analytics according to certain embodiments of this disclosure;

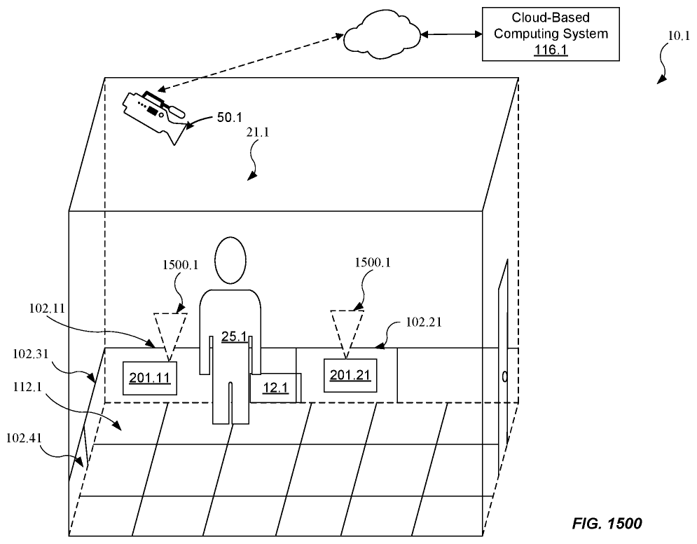

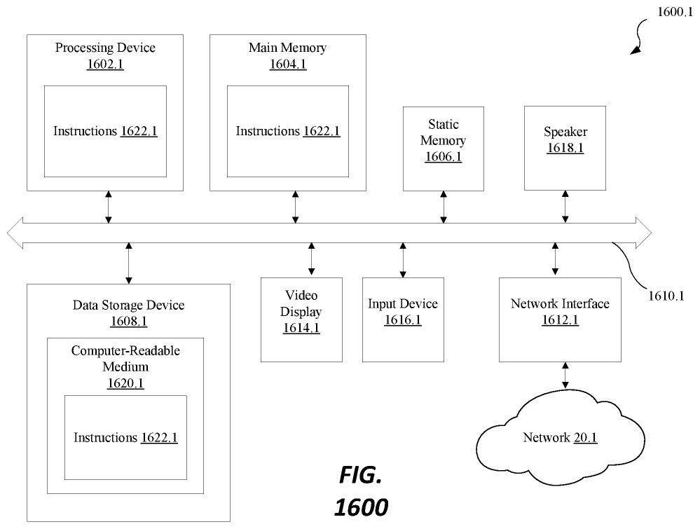

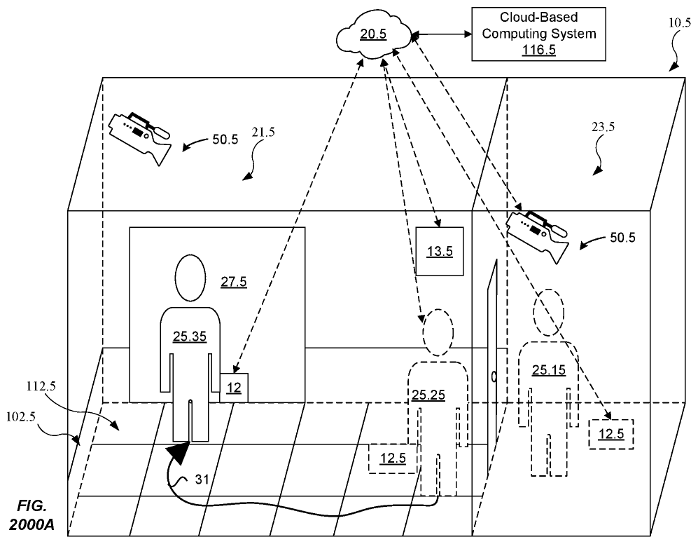

FIG. 15000 illustrates an example for performing, based on a location of a person, one or more actions using one or more devices according to certain embodiments of this disclosure;

FIG. 16000 illustrates an example of a method for performing an action based on a location of a person according to certain embodiments of this disclosure;

FIG. 17000 illustrates an example of a method for monitoring a path of a person after determining their location relative to an object according to certain embodiments of this disclosure;

FIG. 18000 illustrates an example of a method for determining, based on data received from moulding section and smart floor tiles, a distance from a location of a person to a location of an object according to certain embodiments of this disclosure; and

FIG. 19000 illustrates an example computer system according to embodiments of this disclosure.

### NOTATION AND NOMENCLATURE

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

Various terms are used to refer to particular system components. Different entities may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer or section from another region, layer or section. Terms such as “first,” “second,” and other numerical terms, when used herein, do not imply a sequence or order unless clearly indicated by the context. Thus, a first element, component, region, layer or section discussed below could be termed a second element, component, region, layer or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Spatially relative terms, such as “inner,” “outer,” “beneath,” “below,” “lower,” “above,” “upper,” “top,” “bottom,” and the like, may be used herein. These spatially relative terms can be used for ease of description to describe one element's or feature's relationship to another element(s) or feature(s) as illustrated in the figures. The spatially relative terms may also be intended to encompass different orientations of the device in use, or operation, in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptions used herein interpreted accordingly.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), solid state drives (SSDs), flash memory, or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

The term “moulding” may be spelled as “molding” herein.

### DETAILED DESCRIPTION

The following discussion is directed to various embodiments of the disclosed subject matter. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

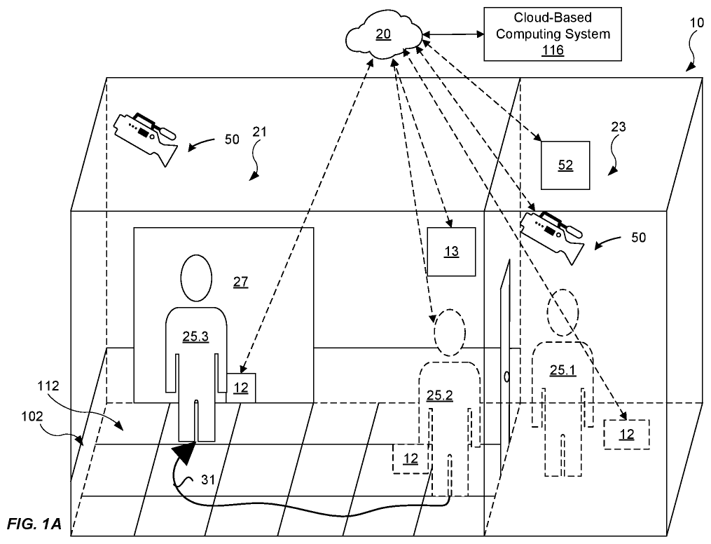

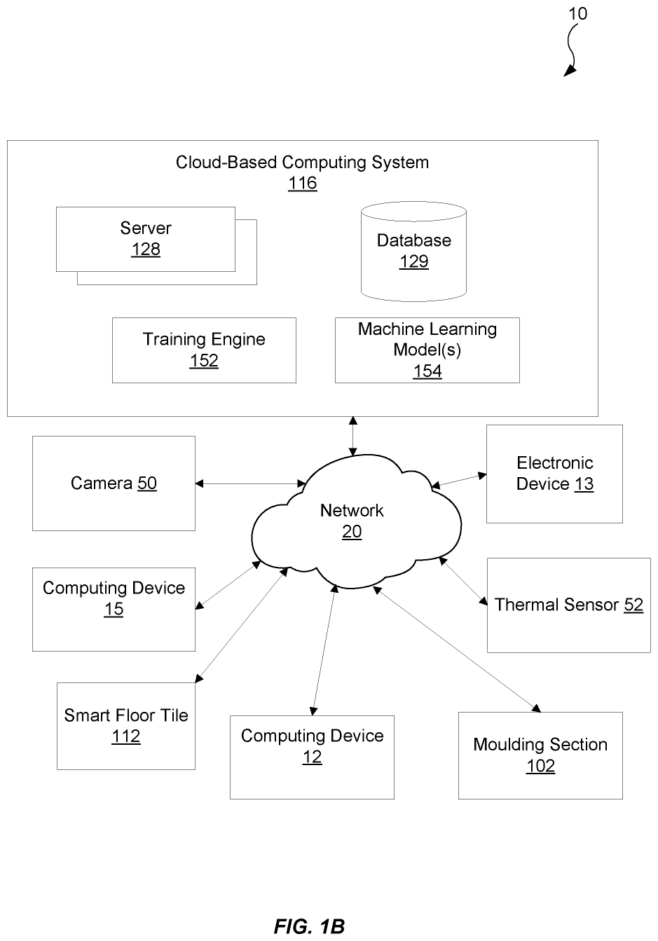

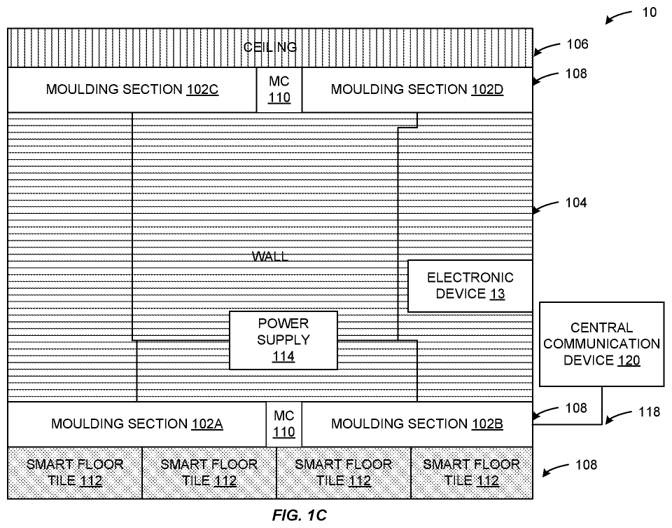

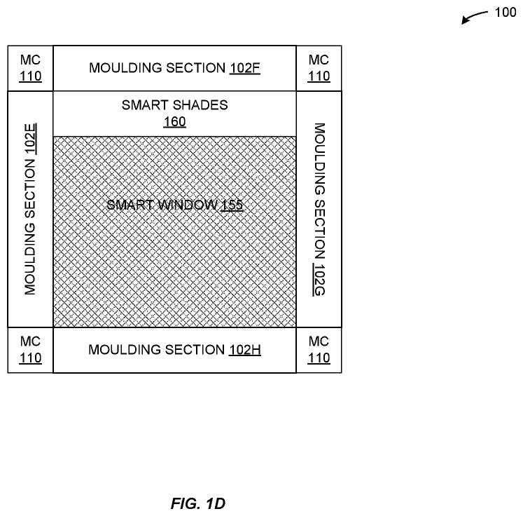

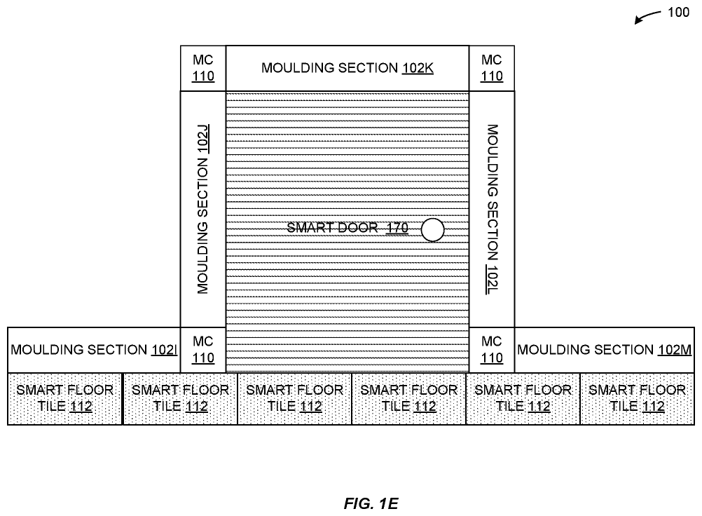

FIGS. 1A through 18, discussed below, and the various embodiments used to describe the principles of this disclosure in this patent document are by way of illustration only and should not be construed in any way to limit the scope of the disclosure.

Embodiments as disclosed herein relate to path analytics for objects in a physical space. For example, the physical space may be a hospital, nursing home, convention center, hotel, or any suitable physical space where people move (e.g., walk, use a wheel chair or motorized cart, etc.) around in a path. Certain locations may be more prone to foot traffic and/or more likely for people to attend due to their proximity to certain other objects (e.g., lobbies, bathrooms, food courts, entrances, exits, etc.). In some instances, certain locations may be more likely for people to attend based on the layout of the physical space and/or the way other locations are arranged in the physical space.

It may be desirable to engage in contact tracing of diseases and disease symptoms at certain locations. For example, it may be beneficial to determine the paths of people that have been or may in the future be determined to have been infected with an infectious disease. It may be desirable to determine the paths of the people in the physical space to better understand which locations are at a higher risk for transmission of diseases. It may be desirable to understand the amounts of time that certain people spend in certain locations or talking to certain people in order to determine the risk of transmission in an interaction. The path analytics may enable determining where to locate certain services in order to reduce risk of transmission of infectious diseases. For example, it may be desirable to separate particularly popular vendors in food courts to spread out the crowds. It may also be desirable to understand where people tend to gather without following social distancing guidelines in order to direct security or supervisory personnel to break up groups or enforce social distancing guidelines. To that end, it may be beneficial to determine the paths of people and which locations in a physical space are more likely to be attended to enable contact tracing or recommend solutions or actions to take in order to reduce the probability of transmission of infectious diseases.

To enable path analytics, some embodiments of the present disclosure may utilize smart floor tiles that are disposed in a physical space where people may move around. For example, the smart floor tiles may be installed in a floor of a convention hall where vendors display objects at booths in certain zones, in a hospital, or in a nursing home. The smart floor tiles may be capable of measuring data (e.g., pressure) associated with footsteps of the people and transmitting the measured data to a cloud-based computing system that analyzes the measured data. In some embodiments, moulding sections, a thermal sensor, and/or a camera may be used to measure the data and/or supplement the data measured by the smart floor tiles. The accuracy of the measurements pertaining to the path of the people may be improved using the smart floor tiles as they measure the physical pressure of the footsteps of the person to track the path of the person and/or other gait characteristics (e.g., width of feet, speed of gait, amount of time spent at certain locations, etc.).

Further, the paths of the people may be correlated with other information, such as job titles of the people, age of the people, gender of the people, employers of the people, detected temperatures of the people, observed labored breathing, and the like. This information may be retrieved from a third party data source and/or data source internal to the cloud-based computing system (e.g., a thermal camera or sensor). For example, the cloud-based computing system may be communicatively coupled with one or more web services (e.g., application programming interfaces) that provide the information to the cloud-based computing system.

The paths that are generated for the people may be overlaid on a virtual representation of the physical space including and/or excluding graphics representing the zones, booths located in the zones, and/or objects displayed in the booths in the physical space. All of the paths of all of the people that move around the physical space during an event, for example, may be overlaid on each other on a user interface presented on a computing device. In some embodiments, a user may select to filter the paths that are presented to just paths of people having a certain job title, to a longest path, to paths that indicate the people visited certain booths, to paths that spent a certain amount of time at a particular zone and/or booth, and the like. The filtering may be performed using any suitable criteria. Accordingly, the disclosed techniques may improve the user's experience using a computing device because an improved user interface that presents desired paths may be provided to the user such that path analytics are enhanced.

The enhanced path analytics may enable the user to make a better determination regarding the layout of facilities. Further, in some embodiments, the cloud-based computing system may analyze the paths and provide contact tracing of people or other living creatures (e.g., a cat or dog, both of which could be potential disease vectors in the physical space. For example, if a person has an elevated temperature, then the cloud-based computing system may recommend that certain other people that person has been in contact with be tested or quarantined.

Barring unforeseeable changes in human locomotion, humans can be expected to generate measurable interactions with buildings through their footsteps on buildings' floors. In some embodiments the smart floor tiles may help realize the potential of a “smart building” by providing, amongst other things, control inputs for a building's environmental control systems using directional occupancy sensing based on occupants' interaction with building surfaces, including, without limitation, floors, interaction with a physical space including their location relative to moulding sections, and climate and airflow systems. Such environmental control systems could act to isolate at risk individuals to reduce the probability of transmission (i.e., by reducing stagnant air around at-risk persons or by placing at-risk persons in isolated air circuits).

The moulding sections, may include a crown moulding, a baseboard, a shoe moulding, a door casing, and/or a window casing, that are located around a perimeter of a physical space. The moulding sections may be modular in nature in that the moulding sections may be various different sizes and the moulding sections may be connected with moulding connectors. The moulding connectors may be configured to maintain conductivity between the connected moulding sections. To that end, each moulding section may include various components, such as electrical conductors, sensors, processors, memories, network interfaces, and so forth that enable communicating data, distributing power, obtaining moulding section sensor data, and so forth. The moulding sections may use various sensors to obtain moulding section sensor data including the location of objects in a physical space as the objects move around the physical space. The moulding sections may use moulding section sensor data to determine a path of the object in the physical space and/or to control other electronic devices (e.g., smart shades, smart windows, smart doors, HVAC system, smart lights, and so forth) in the smart building. Accordingly, the moulding sections may be in wired and/or wireless communication with the other electronic devices. Further, the moulding sections may be in electrical communication with a power supply. The moulding sections may be powered by the power supply and may distribute power to smart floor tiles that may also be in electrical communication with the moulding sections.

A camera may provide a livestream of video data and/or image data to the cloud-based computing system. The camera may be a thermal camera capable of detecting temperatures of objects. The data from the camera may be used to identify certain people in a room and/or track the path of the people in the room. The data from the camera may be used to determine probability of a person being infected (e.g., elevated body temperature) with an infectious disease (e.g., COVID-19). Further, the data may be used to monitor one or more parameters pertaining to a gait of the person to aid in the path analytics. For example, facial recognition may be performed using the data from the camera to identify a person when they first enter a physical space and correlate the identity of the person with the person's path when the person begins to walk on the smart floor tiles.

The cloud-based computing system may monitor one or more parameters of the person based on the measured data from the smart floor tiles, the moulding sections, and/or the camera. The one or more parameters may be associated with the gait of the person and/or the path of the person. Based on the one or more parameters, the cloud-based computing system may determine paths of people in the physical space. The cloud-based computing system may perform any suitable analysis of the paths of the people.

In addition, a technical problem may include determining, from a distal location, when people are in contact with each other and/or within a certain proximity to each other in a physical space. This technical problem is exacerbated if the people in the physical space are not carrying a mobile device that is capable of providing location services. Even when the people are carrying mobile devices, the quality of a signal (e.g., WiFi or cellular) may be poor, which may lead to faulty or inaccurate determinations of whether the people come within a certain proximity to each other.

In addition, a technical problem may include determining, from a distal time and location, how long of an interaction has occurred between two objects (e.g., a doctor and a patient). This technical problem is exacerbated if the people in the physical space are not carrying a mobile device that is capable of providing location services. Even when the people are carrying mobile devices, the quality of a signal (e.g., WiFi or cellular) may be poor, which may lead to faulty or inaccurate determinations of whether the people come within a certain proximity to each other. This technical problem is further exacerbated if the use of cameras or other recording devices are undesirable for any number of reasons (e.g., network bandwidth usage of cameras, technical difficulties and processing power required to properly determine proximity of two objects on a camera, privacy concerns associated with doctor and patient discussions, etc.).

Accordingly, in some embodiments, the present disclosure may provide a technical solution by enabling accurately determining (e.g., via a distal location using a server) when people are in contact with each other and/or within a certain proximity to each other in a physical space. To enable such accurate determination, some embodiments include using measured data from the smart floor tiles, the moulding sections, and/or the camera. Further, thermal data obtained from a thermal sensor in the physical space may determine a temperature of each of the people in the physical space to determine if they exhibit a symptom of a particular disease. The thermal data may be used alone or in conjunction with the measured data to perform a preventative action.

Turning now to the figures, FIGS. 1A-1E illustrate various example configurations of components of a system 10 according to certain embodiments of this disclosure. FIG. 1A visually depicts components of the system in a first room 21 and a second room 23 and FIG. 1B depicts a high-level component diagram of the system 10. For purposes of clarity, FIGS. 1A and 1B are discussed together below.

The first room 21, in this example, is a building that a person 25 is visiting. The first room 21 may be any suitable room that includes a floor capable of being equipped with smart floor tiles 112, moulding sections 102, a camera 50, and/or a thermal sensor 52. The second room 23, in this example, is a entry station or lobby.

When the person initially arrives at the building, the person 25.1 may check in and/or register for entry to the first room 21. As depicted, the person may carry a computing device 12, which may be a smartphone, a laptop, a tablet, a pager, a card, or any suitable computing device. The person 25.1 may use the computing device 12 to check in to the building. For example, the person may 25.1 may swipe the computing device 12 or place it next to a reader that extracts data and sends the data to the cloud-based computing system 116. The data may include an identity of the person 25.1. The reception of the data at the cloud-based computing system 116 may be referred to as an initiation event of a path of an object (e.g., person 25.1) in the physical space (e.g., first room 21) at a first time in a time series. In some embodiments, a camera 50 may send data to the cloud-based computing system 116 that performs facial recognition techniques to determine the identity of the person 25.1. In some embodiments, the thermal sensor 50 may send data to the cloud-based computing system 116 that performs temperature checks against a reference temperature value to determine the probability that the person 25.1 may be infected. Receiving the data from the camera 50 and/or the thermal sensor 52 may also be referred to as an initiation event herein.

Subsequently to the initiation event occurring, the cloud-based computing system 116 may receive data from a first smart floor tile 112 that the person 25.2 steps on at a second time (subsequent to the first time in the time series). The data from the first smart floor tile 112 may occur at a location event that includes an initial location of the person in the physical space. The cloud-based computing device may correlate the initiation event and the initial location to generate a starting point of a path of the person 25.2 in the first room 21.

The person 25.3 may walk around the first room 21 to visit a target location 27. The smart floor tiles 112 may be continuously or continually transmitting measurement data to the cloud-based computing system 116 as the person 25.3 walks from the entrance of the first room 21 to the target location 27. The cloud-based computing system 116 may generate a path 31 of the person 25.3 through the first room 21.

The first room 21 may also include at least one electronic device 13, which may be any suitable electronic device, such as a smart thermostat, smart vacuum, smart light, smart speaker, smart electrical outlet, smart hub, smart appliance, smart television, etc.

Each of the smart floor tiles 112, moulding sections 102, camera 50, thermal sensor 52, computing device 12, and/or electronic device 13 may be capable of communicating, either wirelessly and/or wired, with the cloud-based computing system 116 via a network 20. As used herein, a cloud-based computing system refers, without limitation, to any remote or distal computing system accessed over a network link. Each of the smart floor tiles 112, moulding sections 102, camera 50, computing device 12, and/or electronic device 13 may include one or more processing devices, memory devices, and/or network interface devices.

The network interface devices of the smart floor tiles 112, moulding sections 102, camera 50, thermal sensor 52, computing device 12, and/or electronic device 13 may enable communication via a wireless protocol for transmitting data over short distances, such as Bluetooth, ZigBee, near field communication (NFC), etc. Additionally, the network interface devices may enable communicating data over long distances, and in one example, the smart floor tiles 112, moulding sections 102, camera 50, thermal sensor 52, computing device 12, and/or electronic device 13 may communicate with the network 20. Network 20 may be a public network (e.g., connected to the Internet via wired (Ethernet) or wireless (WiFi)), a private network (e.g., a local area network (LAN), wide area network (WAN), virtual private network (VPN)), or a combination thereof.

The computing device 12 may be any suitable computing device, such as a laptop, tablet, smartphone, or computer. The computing device 12 may include a display that is capable of presenting a user interface. The user interface may be implemented in computer instructions stored on a memory of the computing device 12 and/or computing device 15 and executed by a processing device of the computing device 12. The user interface may be a stand-alone application that is installed on the computing device 12 or may be an application (e.g., website) that executes via a web browser.

The user interface may be generated by the cloud-based computing system 116 and may present various paths of people in the first room 21 on the display screen. The user interface may include various options to filter the paths of the people based on criteria. Also, the user interface may present recommended locations for certain objects in the first room 21. The user interface may be presented on any suitable computing device. For example, computing device 15 may receive and present the user interface to a person interested in the path analytics provided using the disclosed embodiments. The computing device 15 may be any suitable computing device, such as a laptop, tablet, smartphone, or computer.

In some embodiments, the cloud-based computing system 116 may include one or more servers 128 that form a distributed, grid, and/or peer-to-peer (P2P) computing architecture. Each of the servers 128 may include one or more processing devices, memory devices, data storage, and/or network interface devices. The servers 128 may be in communication with one another via any suitable communication protocol. The servers 128 may receive data from the smart floor tiles 112, moulding sections 102, the camera 50, and/or the thermal sensor 52 and monitor a parameter pertaining to a gait of the person 25 based on the data. For example, the data may include pressure measurements obtained by a sensing device in the smart floor tile 112 or temperature of the person 25. The pressure measurements may be used to accurately track footsteps of the person 25, walking paths of the person 25, gait characteristics of the person 25, walking patterns of the person 25 throughout each day, and the like. The servers 128 may determine an amount of gait deterioration based on the parameter. The servers 128 may determine whether a propensity for a fall event for the person 25 satisfies a threshold propensity condition based on (i) the amount of gait deterioration satisfying a threshold deterioration condition, or (ii) the amount of gait deterioration satisfying the threshold deterioration condition within a threshold time period. If the propensity for the fall event for the person 25 satisfies the threshold propensity condition, the servers 128 may select one or more interventions to perform for the person 25 to prevent the fall event from occurring and may perform the one or more selected interventions. The servers 128 may use one or more machine learning models 154 trained to monitor the parameter pertaining to the gait of the person 25 based on the data, determine the amount of gait deterioration based on the parameter, and/or determine whether the propensity for the fall event for the person satisfies the threshold propensity condition.

In some embodiments, the cloud-based computing system 116 may include a training engine 152 and/or the one or more machine learning models 154. The training engine 152 and/or the one or more machine learning models 154 may be communicatively coupled to the servers 128 or may be included in one of the servers 128. In some embodiments, the training engine 152 and/or the machine learning models 154 may be included in the computing device 12, computing device 15, and/or electronic device 13.

The one or more of machine learning models 154 may refer to model artifacts created by the training engine 152 using training data that includes training inputs and corresponding target outputs (correct answers for respective training inputs). The training engine 152 may find patterns in the training data that map the training input to the target output (the answer to be predicted), and provide the machine learning models 154 that capture these patterns. The set of machine learning models 154 may comprise, e.g., a single level of linear or non-linear operations (e.g., a support vector machine [SVM]) or a deep network, i.e., a machine learning model comprising multiple levels of non-linear operations. Examples of such deep networks are neural networks including, without limitation, convolutional neural networks, recurrent neural networks with one or more hidden layers, and/or fully connected neural networks.

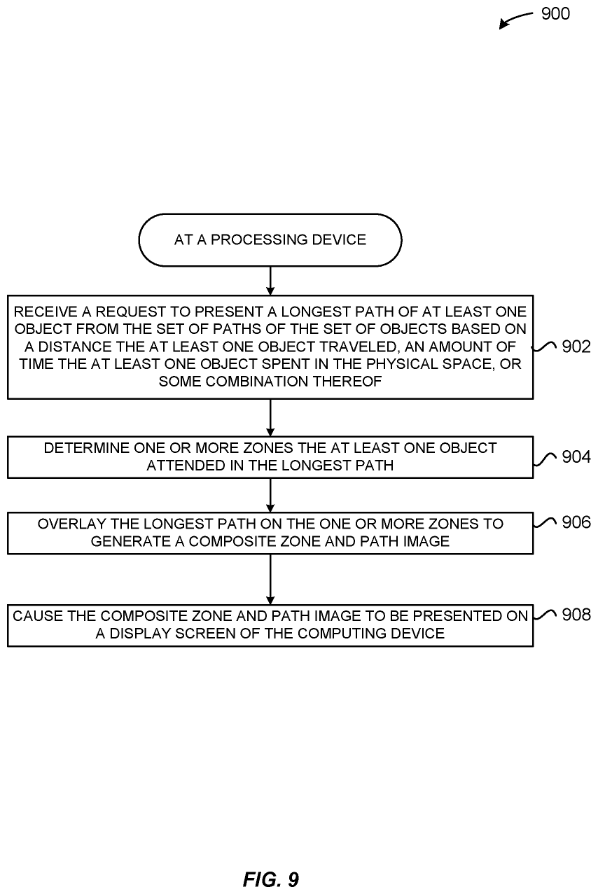

In some embodiments, the training data may include inputs of parameters (e.g., described below with regards to FIG. 9), variations in the parameters, variations in the parameters within a threshold time period, or some combination thereof and correlated outputs of locations of objects to be placed in the first room 21 based on the parameters. That is, in some embodiments, there may be a separate respective machine learning model 154 for each individual parameter that is monitored. The respective machine learning model 154 may output a recommended location for an object based on the parameters (e.g., amount of time people spend at certain locations, paths of people, etc.).

In some embodiments, the cloud-based computing system 116 may include a database 129. The database 129 may store data pertaining to paths of people (e.g., a visual representation of the path, identifiers of the smart floor tiles 112 the person walked on, the amount of time the person stands on each smart floor tile 112 (which may be used to determine an amount of time the person spends at certain booths), and the like), identities of people, recorded temperatures of people, job titles of people, employers of people, age of people, gender of people, residential information of people, and the like. In some embodiments, the database 129 may store data generated by the machine learning models 154, such as recommended locations for objects in the first room 21. Further, the database 129 may store information pertaining to the first room 21, such as the type and location of objects displayed in the first room 21, the booths included in the first room 21, the zones (e.g., boundaries) including the locations the first room (e.g., food courts, bathrooms, etc.) and the like. The database 129 may also store information pertaining to the smart floor tile 112, moulding section 102, the camera 50, and/or the thermal sensor 52, such as device identifiers, addresses, locations, and the like. The database 129 may store paths for people that are correlated with an identity of the person 25. The database 129 may store a map of the first room 21 including the smart floor tiles 112, moulding sections 102, camera 50, any booths 27, and so forth. The database 129 may store video data of the first room 21. The training data used to train the machine learning models 154 may be stored in the database 129.

The camera 50 may be any suitable camera capable of obtaining data including video and/or images and transmitting the video and/or images to the cloud-based computing system 116 via the network 20. The camera 50 may be a thermal (i.e., infrared) camera. The data obtained by the camera 50 may include timestamps for the video and/or images. In some embodiments, the cloud-based computing system 116 may perform computer vision to extract high-dimensional digital data from the data received from the camera 50 and produce numerical or symbolic information. The numerical or symbolic information may represent the parameters monitored pertaining to the path of the person 25 monitored by the cloud-based computing system 116. The video data obtained by the camera 50 may be used for facial recognition of the person 25.

The thermal sensor 52 may be any suitable device (including a thermal camera) capable of detecting temperature information and transmitting the temperature information to the cloud-based computing system 116 via the network 20. The data obtained by the temperature sensor 52 may include timestamps for the video and/or images.

FIGS. 1C-1E depict various example configurations of smart floor tiles 112, and/or moulding sections 102 according to certain embodiments of this disclosure. FIG. 1C depicts an example system 10 that is used in a physical space of a smart building (e.g., care facility). The depicted physical space includes a wall 104, a ceiling 106, and a floor 108 that define a room. Numerous moulding sections 102A, 102B, 102C, and 102D are disposed in the physical space. For example, moulding sections 102A and 102B may form a baseboard or shoe moulding that is secured to the wall 108 and/or the floor 108. Moulding sections 102C and 102D may for a crown moulding that is secured to the wall 108 and/or the ceiling 106. Each moulding section 102A may have different shapes and/or sizes.

The moulding sections 102 may each include various components, such as electrical conductors, sensors, processors, memories, network interfaces, and so forth. The electrical conductors may be partially or wholly enclosed within one or more of the moulding sections. For example, one electrical conductor may be a communication cable that is partially enclosed within the moulding section and exposed externally to the moulding section to electrically couple with another electrical conductor in the wall 108. In some embodiments, the electrical conductor may be communicably connected to at least one smart floor tile 112. In some embodiments, the electrical conductor may be in electrical communication with a power supply 114. In some embodiments, the power supply 114 may provide electrical power that is in the form of mains electricity general-purpose alternating current. In some embodiments, the power supply 114 may be a battery, a generator, or the like.

In some embodiments, the electrical conductor is configured for wired data transmission. To that end, in some embodiments the electrical conductor may be communicably coupled via cable 118 to a central communication device 120 (e.g., a hub, a modem, a router, etc.). Central communication device 120 may create a network, such as a wide area network, a local area network, or the like. Other electronic devices 13 may be in wired and/or wireless communication with the central communication device 120. Accordingly, the moulding section 102 may transmit data to the central communication device 120 to transmit to the electronic devices 13. The data may be control instructions that cause, for example, an the electronic device 13 to change a property. In some embodiments, the moulding section 102A may be in wired and/or wireless communication connection with the electronic device 13 without the use of the central communication device 120 via a network interface and/or cable. The electronic device 13 may be any suitable electronic device capable of changing an operational parameter in response to a control instruction.

In some embodiments, the electrical conductor may include an insulated electrical wiring assembly. In some embodiments, the electrical conductor may include a communications cable assembly. The moulding sections 102 may include a flame-retardant backing layer. The moulding sections 102 may be constructed using one or more materials selected from: wood, vinyl, rubber, fiberboard, metal, plastic, and wood composite materials.

The moulding sections may be connected via one or more moulding connectors 110. A moulding connector 110 may enhance electrical conductivity between two moulding sections 102 by maintaining the conductivity between the electrical conductors of the two moulding sections 102. For example, the moulding connector 110 may include contacts and its own electrical conductor that forms a closed circuit when the two moulding sections are connected with the moulding connector 110. In some embodiments, the moulding connectors 110 may include a fiber optic relay to enhance the transfer of data between the moulding sections 102. It should be appreciated that the moulding sections 102 are modular and may be cut into any desired size to fit the dimensions of a perimeter of a physical space. The various sized portions of the moulding sections 102 may be connected with the moulding connectors 110 to maintain conductivity.

Moulding sections 102 may utilize a variety of sensing technologies, such as proximity sensors, optical sensors, membrane switches, pressure sensors, and/or capacitive sensors, to identify instances of an object proximate or located near the sensors in the moulding sections and to obtain data pertaining to a gait of the person 25. Proximity sensors may emit an electromagnetic field or a beam of electromagnetic radiation (infrared, for instance), and identify changes in the field or return signal. The object being sensed may be any suitable object, such as a human, an animal, a robot, furniture, appliances, and the like. Sensing devices in the moulding section may generate moulding section sensor data indicative of gait characteristics of the person 25, location (presence) of the person 25, the timestamp associated with the location of the person 25, and so forth.

The moulding section sensor data may be used alone or in combination with tile impression data generated by the smart floor tiles 112 and/or image data generated by the camera 50 to perform path analytics for people. For example, the moulding section sensor data may be used to determine a control instruction to generate and to transmit to an electric device 13 and/or the smart floor tile 102A. The control instruction may include changing an operational parameter of the electronic device 13 based on the moulding section sensor data. The control instruction may include instructing the smart floor tile 112 to reset one or more components based on an indication in the moulding section sensor data that the one or more components is malfunctioning and/or producing faulty results. Further, the moulding sections 102 may include a directional indicator (e.g., light) that emits different colors of light, intensities of light, patterns of light, etc. based on path analytics of the cloud-based computing system 116.

In some embodiments, the moulding section sensor data can be used to verify the impression tile data and/or image data of the camera 50 is accurate for generating and analyzing paths of people. Such a technique may improve accuracy of the path analytics. Further, if the moulding section sensor data, the impression tile data, and/or the image data do not align (e.g., the moulding section sensor data does not indicate a path of a person and impression tile data indicates a path of the person), then further analysis may be performed. For example, tests can be performed to determine if there are defective sensors at the corresponding smart floor tile 112 and/or the corresponding moulding section 102 that generated the data. Further, control actions may be performed such as resetting one or more components of the moulding section 102 and/or the smart floor tile 112. In some embodiments, preference to certain data may be made by the cloud-based computing system 116. For example, in one embodiment, preference for the impression tile data may be made over the moulding section sensor data and/or the image data, such that if the impression tile data differs from the moudling section sensor data and/or the image data, the impression tile data is used to perform path analytics.

FIG. 1D illustrates another configuration of the moulding sections 102. In this example, the moulding sections 102E-102H surround a border of a smart window 155. The moulding sections 102 are connected via the moulding connector 110. As may be appreciated, the modular nature of the moulding sections 102 with the moulding connectors 110 enables forming a square around the window. Other shapes may be formed using the moulding sections 102 and the moulding connectors 110.

The moulding sections 102 may be electrically and/or communicably connected to the smart window 155 via electrical conductors and/or interfaces. The moulding sections 102 may provide power to the smart window 155, receive data from the smart window 155, and/or transmit data to the smart window 155. One example smart window includes the ability to change light properties using voltage that may be provided by the moulding sections 102. The moulding sections 102 may provide the voltage to control the amount of light let into a room based on path analytics. For example, if the moulding section sensor data, impression tile data, and/or image data indicates a portion of the first room 21 includes a lot of people, the cloud-based computing system 116 may perform an action by causing the moulding sections 102 to instruct the smart window 155 to change a light property to allow light into the room. In some instances the cloud-based computing system 116 may communicate directly with the smart window 155 (e.g., electronic device 13).

In some embodiments, the moulding sections 102 may use sensors to detect when the smart window 155 is opened. The moulding sections 102 may determine whether the smart window 155 opening is performed at an expected time (e.g., when a home owner is at home) or at an unexpected time (e.g., when the home owner is away from home). The moulding sections 102, the camera 50, and/or the smart floor tile 112 may sense the occupancy patterns of certain objects (e.g., people) in the space in which the moulding sections 102 are disposed to determine a schedule of the objects. The schedule may be referenced when determining if an undesired opening (e.g., break-in event) occurs and the moulding sections 102 may be communicatively to an alarm system to trigger the alarm when the certain event occurs.

The schedule may also be referenced when determining a medical condition of the person 25. For example, if the schedule indicates that the person 25 went to the bathroom a certain number of times (e.g., 10) within a certain time period (e.g., 1 hour), the cloud-based computing system 116 may determine that the person has a urinary tract infection (UTI) and may perform an intervention, such as transmitting a message to the computing device 12 of the person 25. The message may indicate the potential UTI and recommend that the person 25 schedules an appointment with a medical personnel.

As depicted, at least moulding section 102F is electrically and/or communicably coupled to smart shades 160. Again, the cloud-based computing system 116 may cause the moulding section 102F to control the smart shades 160 to extend or retract to control the amount of light let into a room. In some embodiments, the cloud-based computing system 116 may communicate directly with the smart shades 160.

FIG. 1E illustrates another configuration of the moulding sections 102 and smart floor tiles 112. In this example, the moulding sections 102E-102H surround a majority of a border of a smart door 170. The moulding sections 102J, 102K, and 102L and/or the smart floor tile 112 may be electrically and/or communicably connected to the smart door 170 via electrical conductors and/or interfaces. The moulding sections 102 and/or smart floor tiles 112 may provide power to the smart door 170, receive data from the smart door 170, and/or transmit data to the smart door 170. In some embodiments, the moulding sections 102 and/or smart floor tiles 112 may control operation of the smart door 170. For example, if the moulding section sensor data and/or impression tile data indicates that no one is present in a house for a certain period of time, the moulding sections 102 and/or smart floor tiles 112 may determine a locked state of the smart door 170 and generate and transmit a control instruction to the smart door 170 to lock the smart door 170 if the smart door 170 is in an unlocked state.

In another example, the moulding section sensor data, impression tile data, and/or the image data may be used to generate gait profiles for people in a smart building (e.g., care facility). When a certain person is in the room near the smart door 170, the cloud-based computing device 116 may detect that person's presence based on the data received from the smart floor tiles, moulding sections 102, and/or camera 50. In some embodiments, if the person 25 is detected near the smart door 170, the cloud-based computing system 116 may determine whether the person 25 has a particular medical condition (e.g., alzheimers) and/or a flag is set that the person should not be allowed to leave the smart building. If the person is detected near the smart door 170 and the person 25 has the particular medical condition and/or the flag set, then the cloud-based computing system 116 may cause the moulding sections 102 and/or smart floor tiles 112 to control the smart door 170 to lock the smart door 170. In some embodiments, the cloud-based computing system 116 may communicate directly with the smart door 170 to cause the smart door 170 to lock.

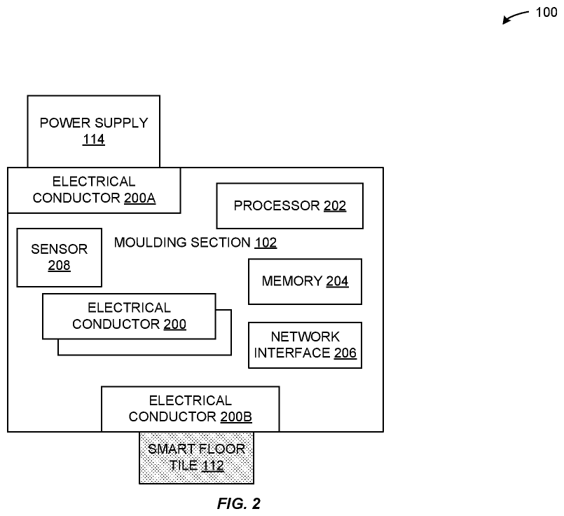

FIG. 2 illustrates an example component diagram of a moulding section 102 according to certain embodiments of this disclosure. As depicted, the moulding section 102 includes numerous electrical conductors 200, a processor 202, a memory 204, a network interface 206, and a sensor 208. More or fewer components may be included in the moulding section 102. The electrical conductors may be insulated electrical wiring assemblies, communications cable assemblies, power supply assemblies, and so forth. As depicted, one electrical conductor 200A may be in electrical communication with the power supply 114, and another electrical conductor 200B may be communicably connected to at least one smart floor tile 112.

In various embodiments, the moulding section 102 further comprises a processor 202. In the non-limiting example shown in FIG. 2, processor 202 is a low-energy microcontroller, such as the ATMEGA328P by Atmel Corporation. According to other embodiments, processor 202 is the processor provided in other processing platforms, such as the processors provided by tablets, notebook or server computers.

In the non-limiting example shown in FIG. 2, the moulding section 102 includes a memory 204. According to certain embodiments, memory 204 is a non-transitory memory containing program code to implement, for example, generation and transmission of control instructions, networking functionality, the algorithms for generating and analyzing locations, presence, paths, and/or tracks, and the algorithms for performing path analytics as described herein.

Additionally, according to certain embodiments, the moulding section 102 includes the network interface 206, which supports communication between the moulding section 102 and other devices in a network context in which smart building control using directional occupancy sensing and path analytics is being implemented according to embodiments of this disclosure. In the non-limiting example shown in FIG. 2, network interface 206 includes circuitry 635 for sending and receiving data using Wi-Fi, including, without limitation at 900 MHz, 2.8 GHz and 5.0 GHz. Additionally, network interface 206 includes circuitry, such as Ethernet circuitry 640 for sending and receiving data (for example, smart floor tile data) over a wired connection. In some embodiments, network interface 206 further comprises circuitry for sending and receiving data using other wired or wireless communication protocols, such as Bluetooth Low Energy or Zigbee circuitry. The network interface 206 may enable communicating with the cloud-based computing device 116 via the network 20.

Additionally, according to certain embodiments, network interface 206 which operates to interconnect the moulding device 102 with one or more networks. Network interface 206 may, depending on embodiments, have a network address expressed as a node ID, a port number or an IP address. According to certain embodiments, network interface 206 is implemented as hardware, such as by a network interface card (NIC). Alternatively, network interface 206 may be implemented as software, such as by an instance of the java.net.NetworkInterface class. Additionally, according to some embodiments, network interface 206 supports communications over multiple protocols, such as TCP/IP as well as wireless protocols, such as 3G or Bluetooth. Network interface 206 may be in communication with the central communication device 120 in FIG. 1.

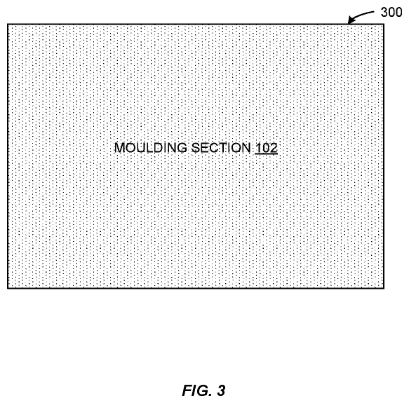

FIG. 3 illustrates an example backside view 300 of a moulding section 102 according to certain embodiments of this disclosure. As depicted by the dots 300, the backside of the moulding section 102 may include a fire-retardant backing layer positioned between the moulding section 102 and the wall to which the moulding section 102 is secured.

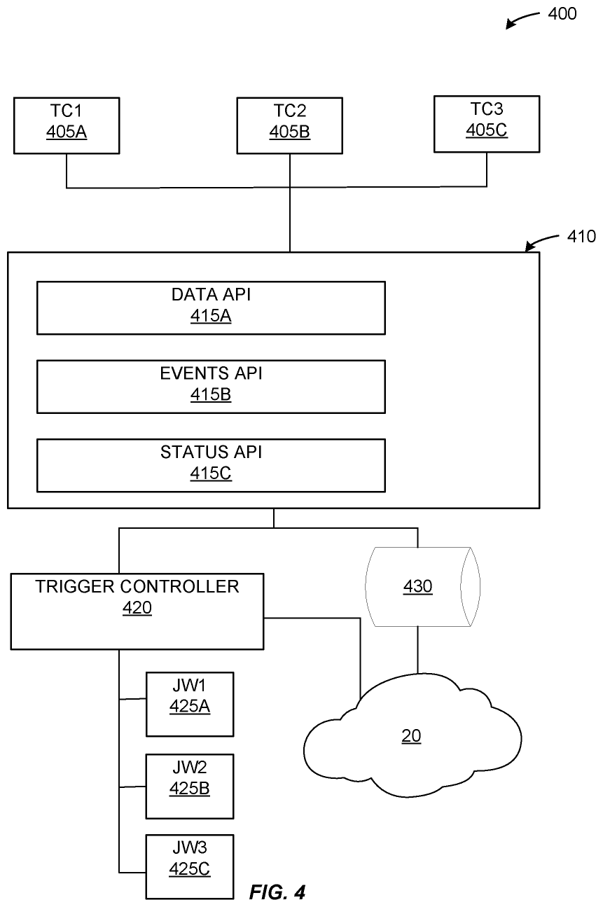

FIG. 4 illustrates a network and processing context 400 for smart building control using directional occupancy sensing and path analytics according to certain embodiments of this disclosure. The embodiment of the network context 400 shown in FIG. 4 is for illustration only and other embodiments could be used without departing from the scope of the present disclosure.

In the non-limiting example shown in FIG. 4, a network context 400 includes one or more tile controllers 405A, 405B and 405C, an API suite 410, a trigger controller 420, job workers 425A-425C, a database 430 and a network 435.

According to certain embodiments, each of tile controllers 405A-405C is connected to a smart floor tile 112 in a physical space. Tile controllers 405A-405C generate floor contact data (also referred to as impression tile data herein) from smart floor tiles in a physical space and transmit the generated floor contact data to API suite 410. In some embodiments, data from tile controllers 405A-405C is provided to API suite 410 as a continuous stream. In the non-limiting example shown in FIG. 4, tile controllers 405A-405C provide the generated floor contact data from the smart floor tile to API suite 410 via the internet. Other embodiments, wherein tile controllers 405A-405C employ other mechanisms, such as a bus or Ethernet connection to provide the generated floor data to API suite 410 are possible and within the intended scope of this disclosure.

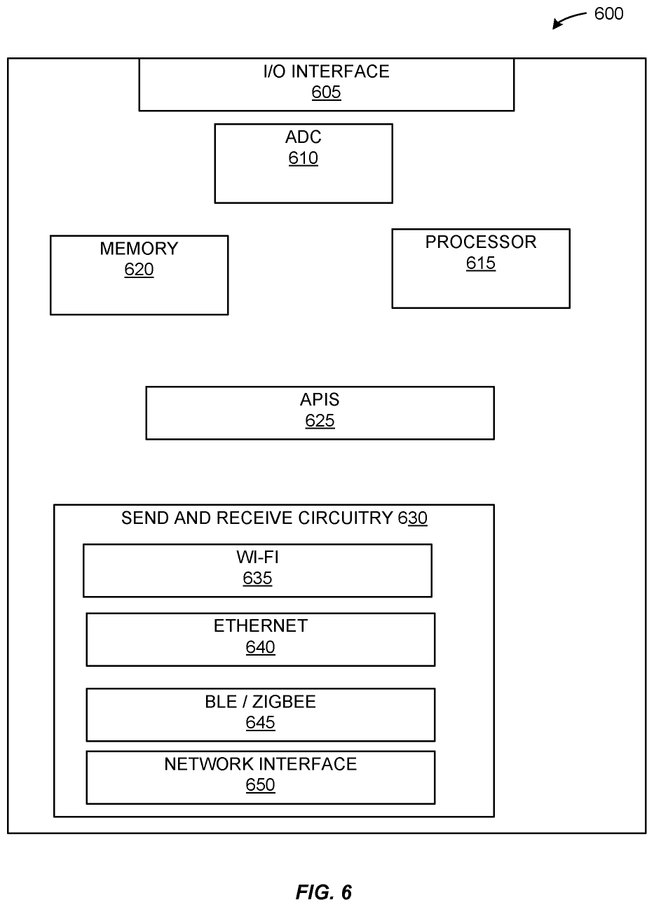

According to some embodiments, API suite 410 is embodied on a server 128 in the cloud-based computing system 116 connected via the internet to each of tile controllers 405A-405C. According to some embodiments, API suite is embodied on a master control device, such as master control device 600 shown in FIG. 6 of this disclosure. In the non-limiting example shown in FIG. 4, API suite 410 comprises a Data Application Programming Interface (API) 415A, an Events API 415B and a Status API 215C.

In some embodiments, Data API 415A is an API for receiving and recording tile data from each of tile controllers 405A-405C. Tile events include, for example, raw, or minimally processed data from the tile controllers, such as the time and data a particular smart floor tile was pressed and the duration of the period during which the smart floor tile was pressed. According to certain embodiments, Data API 415A stores the received tile events in a database such as database 430. In the non-limiting example shown in FIG. 4, some or all of the tile events are received by API suite 410 as a stream of event data from tile controllers 405A-405C, Data API 415A operates in conjunction with trigger controller 420 to generate and pass along triggers breaking the stream of tile event data into discrete portions for further analysis.

According to various embodiments, Events API 415B receives data from tile controllers 405A-405C and generates lower-level records of instantaneous contacts where a sensor of the smart floor tile is pressed and released.

In the non-limiting example shown in FIG. 4, Status API 415C receives data from each of tile controllers 405A-405C and generates records of the operational health (for example, CPU and memory usage, processor temperature, whether all of the sensors from which a tile controller receives inputs is operational) of each of tile controllers 405A-405C. According to certain embodiment, status API 415C stores the generated records of the tile controllers' operational health in database 430.

According to some embodiments, trigger controller 420 operates to orchestrate the processing and analysis of data received from tile controllers 405A-405C. In addition to working with data API 415A to define and set boundaries in the data stream from tile controllers 405A-405C to break the received data stream into tractably sized and logically defined “chunks” for processing, trigger controller 420 also sends triggers to job workers 425A-425C to perform processing and analysis tasks. The triggers comprise identifiers uniquely identifying each data processing job to be assigned to a job worker. In the non-limiting example shown in FIG. 4, the identifiers comprise: 1.) a sensor identifier (or an identifier otherwise uniquely identifying the location of contact); 2.) a time boundary start identifying a time in which the smart floor tile went from an idle state (for example, an completely open circuit, or, in the case of certain resistive sensors, a baseline or quiescent current level) to an active state (a closed circuit, or a current greater than the baseline or quiescent level); and 3.) a time boundary end defining the time in which a smart floor tile returned to the idle state.

In some embodiments, each of job workers 425A-425C corresponds to an instance of a process performed at a computing platform, (for example, cloud-based computing system 116 in FIG. 1) for determining paths and performing an analysis of the paths (e.g., such as filtering paths based on criteria, recommending a location of an object based on the paths, predicting a propensity for a fall event and performing an intervention based on the propensity). Instances of processes may be added or subtracted depending on the number of events or possible events received by API suite 410 as part of the data stream from tile controllers 405A-205C. According to certain embodiments, job workers 425A-425C perform an analysis of the data received from tile controllers 405A-405C, the analysis having, in some embodiments, two stages. A first stage comprises deriving footsteps, and paths, or tracks, from impression tile data. A second stage comprises characterizing those footsteps, and paths, or tracks, to determine gait characteristics of the person 25. The paths and/or gait characteristics may be presented to an online dashboard (in some embodiments, provided by a UI on an electronic device, such as computing device 12 or 15 in FIG. 1) and to generate control signals for devices (e.g., the computing devices 12 and/or 15, the electronic device 15, the moulding sections 102, the camera 50, and/or the smart floor tile 112 in FIG. 1) controlling operational parameters of a physical space where the smart floor impression tile data were recorded.

In the non-limiting example shown in FIG. 4, job workers 425A-425C perform the constituent processes of a method for analyzing smart floor tile impression tile data and/or moulding section sensor data to generate paths, or tracks. In some embodiments, an identity of the person 25 may be correlated with the paths or tracks. For example, if the person scanned an ID badge when entering the physical space, their path may be recorded when the person takes their first step on a smart floor tile and their path may be correlated with an identifier received from scanning the badge. In this way, the paths of various people may be recorded (e.g., in a convention hall). This may be beneficial if certain people have desirable job titles (e.g., chief executive officer (CEO), vice president, president, etc.) and/or work at desirable client entities. For example, in some embodiments, the path of a CEO may be tracked during a convention to determine which booths the CEO stopped at and/or an amount of time the CEO spent at each booth. Such data may be used to determine where to place certain booths in the future. For example, if a booth was visited by a threshold number of people having a certain title for a certain period of time, a recommendation may be generated and presented that recommends relocating the booth to a location in the convention hall that is more easily accessible to foot traffic. Likewise, if it is determined that a booth has poor visitation frequency based on the paths, or tracks, of attendees at the convention, a recommendation may be generated to relocate the booth to another location that is more easily accessible to foot traffic. In some embodiments, the machine learning models 154 may be trained to determine the paths, or tracks, of the people having various job titles and working for desired client entities, analyze their paths (e.g., which location the people visited, how long the people visited those locations, etc.), and generate recommendations.

According to certain embodiments, the method comprises the operations of obtaining impression image data, impression tile data, and/or moulding section sensor data from database 430, cleaning the obtained image data, impression tile data, and/or moulding section sensor data and reconstructing paths using the cleaned data. In some embodiments, cleaning the data includes removing extraneous sensor data, removing gaps between image data, impression tile data, and/or moulding section sensor data caused by sensor noise, removing long image data, impression tile data, and/or moulding section sensor data caused by objects placed on smart floor tiles, by objects placed in front of moulding sections, by objects stationary in image data, by defective sensors, and sorting image data, impression tile data, and/or moulding section sensor data by start time to produce sorted image data, impression tile data, and/or moulding section sensor data. According to certain embodiments, job workers 425A-425C perform processes for reconstructing paths by implementing algorithms that first cluster image data, impression tile data, and/or moulding section sensor data that overlap in time or are spatially adjacent. Next, the clustered data is searched, and pairs of image data, impression tile data, and/or moulding section sensor data that start or end within a few milliseconds of one another are combined into footsteps and/or locations of the object, which are then linked together to form footsteps and/or locations. Footsteps and/or locations are further analyzed and linked to create paths.

According to certain embodiments, database 430 provides a repository of raw and processed image data, smart floor tile impression tile data, and/or moulding section sensor data, as well as data relating to the health and status of each of tile controllers 405A-405C and moulding sections 102. In the non-limiting example shown in FIG. 4, database 430 is embodied on a server machine communicatively connected to the computing platforms providing API suite 410, trigger controller 420, and upon which job workers 425A-425C execute. According to some embodiments, database 430 is embodied on the cloud-based computing system 116 as the database 129.

In the non-limiting example shown in FIG. 4, the computing platforms providing trigger controller 420 and database 430 are communicatively connected to one or more network(s) 20. According to embodiments, network 20 comprises any network suitable for distributing impression tile data, image data, moulding section sensor data, determined paths, determined gait deterioration of a parameter, determine propensity for a fall event, and control signals (e.g., interventions) based on determined propensities for fall events, including, without limitation, the internet or a local network (for example, an intranet) of a smart building.

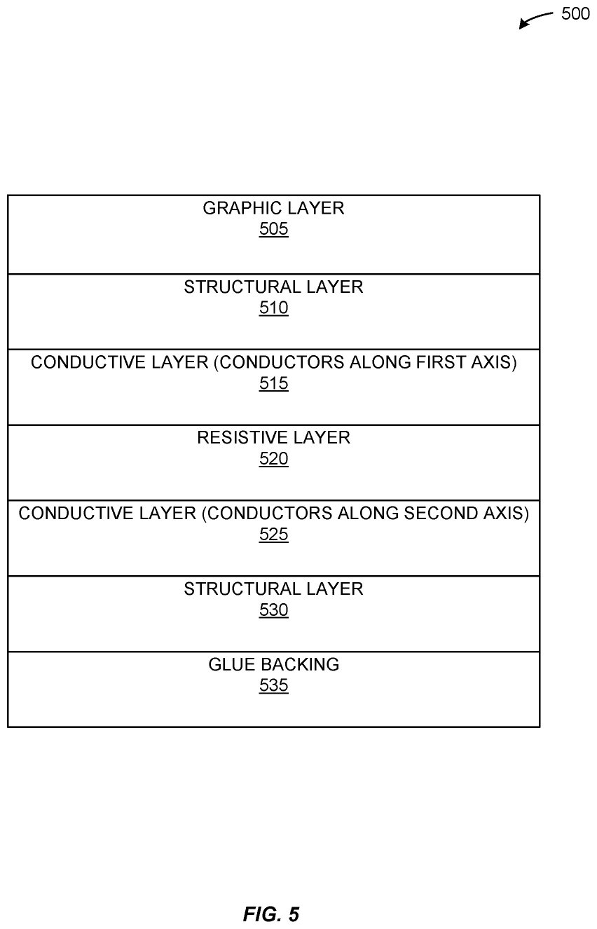

Smart floor tiles utilizing a variety of sensing technologies, such as membrane switches, pressure sensors and capacitive sensors, to identify instances of contact with a floor are within the contemplated scope of this disclosure. FIG. 5 illustrates aspects of a resistive smart floor tile 500 according to certain embodiments of the present disclosure. The embodiment of the resistive smart floor tile 500 shown in FIG. 5 is for illustration only and other embodiments could be used without departing from the scope of the present disclosure.

In the non-limiting example shown in FIG. 5, a cross section showing the layers of a resistive smart floor tile 500 is provided. According to some embodiments, the resistance to the passage of electrical current through the smart floor tile varies in response to contact pressure. From these changes in resistance, values corresponding to the pressure and location of the contact may be determined. In some embodiments, resistive smart floor tile 500 may comprise a modified carpet or vinyl floor tile, and have dimensions of approximately 2′×2′.

According to certain embodiments, resistive smart floor tile 500 is installed directly on a floor, with graphic layer 505 comprising the top-most layer relative to the floor. In some embodiments, graphic layer 505 comprises a layer of artwork applied to smart floor tile 500 prior to installation. Graphic layer 505 can variously be applied by screen printing or as a thermal film.

According to certain embodiments, a first structural layer 510 is disposed, or located, below graphic layer 505 and comprises one or more layers of durable material capable of flexing at least a few thousandths of an inch in response to footsteps or other sources of contact pressure. In some embodiments, first structural layer 510 may be made of carpet, vinyl or laminate material.

According to some embodiments, first conductive layer 515 is disposed, or located, below structural layer 510. According to some embodiments, first conductive layer 515 includes conductive traces or wires oriented along a first axis of a coordinate system. The conductive traces or wires of first conductive layer 515 are, in some embodiments, copper or silver conductive ink wires screen printed onto either first structural layer 510 or resistive layer 520. In other embodiments, the conductive traces or wires of first conductive layer 515 are metal foil tape or conductive thread embedded in structural layer 510. In the non-limiting example shown in FIG. 5, the wires or traces included in first conductive layer 515 are capable of being energized at low voltages on the order of 5 volts. In the non-limiting example shown in FIG. 5, connection points to a first sensor layer of another smart floor tile or to tile controller are provided at the edge of each smart floor tile 500.

In various embodiments, a resistive layer 520 is disposed, or located, below conductive layer 515. Resistive layer 520 comprises a thin layer of resistive material whose resistive properties change under pressure. For example, resistive layer 320 may be formed using a carbon-impregnated polyethylete film.

In the non-limiting example shown in FIG. 5, a second conductive layer 525 is disposed, or located, below resistive layer 520. According to certain embodiments, second conductive layer 525 is constructed similarly to first conductive layer 515, except that the wires or conductive traces of second conductive layer 525 are oriented along a second axis, such that when smart floor tile 500 is viewed from above, there are one or more points of intersection between the wires of first conductive layer 515 and second conductive layer 525. According to some embodiments, pressure applied to smart floor tile 500 completes an electrical circuit between a sensor box (for example, tile controller 425 as shown in FIG. 4) and smart floor tile, allowing a pressure-dependent current to flow through resistive layer 520 at a point of intersection between the wires of first conductive layer 515 and second conductive layer 525. The pressure-dependent current may represent a measurement of pressure and the measurement of pressure may be transmitted to the cloud-based computing system 116.

In some embodiments, a second structural layer 530 resides beneath second conductive layer 525. In the non-limiting example shown in FIG. 5, second structural layer 530 comprises a layer of rubber or a similar material to keep smart floor tile 500 from sliding during installation and to provide a stable substrate to which an adhesive, such as glue backing layer 535 can be applied without interference to the wires of second conductive layer 525.

The foregoing description is purely descriptive and variations thereon are contemplated as being within the intended scope of this disclosure. For example, in some embodiments, smart floor tiles according to this disclosure may omit certain layers, such as glue backing layer 535 and graphic layer 505 described in the non-limiting example shown in FIG. 5.

According to some embodiments, a glue backing layer 535 comprises the bottom-most layer of smart floor tile 500. In the non-limiting example shown in FIG. 5, glue backing layer 535 comprises a film of a floor tile glue.

FIG. 6 illustrates a master control device 600 according to certain embodiments of this disclosure. FIG. 6 illustrates a master control device 600 according to certain embodiments of this disclosure. The embodiment of the master control device 600 shown in FIG. 6 is for illustration only and other embodiments could be used without departing from the scope of the present disclosure.

In the non-limiting example shown in FIG. 6, master control device 600 is embodied on a standalone computing platform connected, via a network, to a series of end devices (e.g., tile controller 405A in FIG. 4) in other embodiments, master control device 600 connects directly to, and receives raw signals from, one or more smart floor tiles (for example, smart floor tile 500 in FIG. 5). In some embodiments, the master control device 600 is implemented on a server 128 of the cloud-based computing system 116 in FIG. 1B and communicates with the smart floor tiles 112, the moulding sections 102, the camera 50, the computing device 12, the computing device 15, and/or the electronic device 13.

According to certain embodiments, master control device 600 includes one or more input/output interfaces (I/O) 605. In the non-limiting example shown in FIG. 6, I/O interface 605 provides terminals that connect to each of the various conductive traces of the smart floor tiles deployed in a physical space. Further, in systems where membrane switches or smart floor tiles are used as mat presence sensors, I/O interface 605 electrifies certain traces (for example, the traces contained in a first conductive layer, such as conductive layer 515 in FIG. 5) and provides a ground or reference value for certain other traces (for example, the traces contained in a second conductive layer, such as conductive layer 525 in FIG. 5). Additionally, I/O interface 605 also measures current flows or voltage drops associated with occupant presence events, such as a person's foot squashing a membrane switch to complete a circuit, or compressing a resistive smart floor tile, causing a change in a current flow across certain traces. In some embodiments, I/O interface 605 amplifies or performs an analog cleanup (such as high or low pass filtering) of the raw signals from the smart floor tiles in the physical space in preparation for further processing.

In some embodiments, master control device 600 includes an analog-to-digital converter (“ADC”) 610. In embodiments where the smart floor tiles in the physical space output an analog signal (such as in the case of resistive smart floor tile), ADC 610 digitizes the analog signals. Further, in some embodiments, ADC 610 augments the converted signal with metadata identifying, for example, the trace(s) from which the converted signal was received, and time data associated with the signal. In this way, the various signals from smart floor tiles can be associated with touch events occurring in a coordinate system for the physical space at defined times. While in the non-limiting example shown in FIG. 6, ADC 610 is shown as a separate component of master control device 600, the present disclosure is not so limiting, and embodiments wherein ADC 610 is part of, for example, I/O interface 605 or processor 615 are contemplated as being within the scope of this disclosure.

In various embodiments, master control device 600 further comprises a processor 615. In the non-limiting example shown in FIG. 6, processor 615 is a low-energy microcontroller, such as the ATMEGA328P by Atmel Corporation. According to other embodiments, processor 615 is the processor provided in other processing platforms, such as the processors provided by tablets, notebook or server computers.

In the non-limiting example shown in FIG. 6, master control device 600 includes a memory 620. According to certain embodiments, memory 620 is a non-transitory memory containing program code to implement, for example, APIs 625, networking functionality and the algorithms for generating and analyzing paths described herein.

Additionally, according to certain embodiments, master control device 600 includes one or more Application Programming Interfaces (APIs) 625. In the non-limiting example shown in FIG. 6, APIs 625 include APIs for determining and assigning break points in one or more streams of smart floor tile data and/or moulding section sensor data and defining data sets for further processing. Additionally, in the non-limiting example shown in FIG. 6, APIs 625 include APIs for interfacing with a job scheduler (for example, trigger controller 420 in FIG. 4) for assigning batches of data to processes for analysis and determination of paths. According to some embodiments, APIs 625 include APIs for interfacing with one or more reporting or control applications provided on a client device. Still further, in some embodiments, APIs 625 include APIs for storing and retrieving image data, smart floor tile data, and/or moulding section sensor data in one or more remote data stores (for example, database 430 in FIG. 4, database 129 in FIG. 1B, etc.).

According to some embodiments, master control device 600 includes send and receive circuitry 630, which supports communication between master control device 600 and other devices in a network context in which smart building control using directional occupancy sensing is being implemented according to embodiments of this disclosure. In the non-limiting example shown in FIG. 6, send and receive circuitry 630 includes circuitry 635 for sending and receiving data using Wi-Fi, including, without limitation at 900 MHz, 2.8 GHz and 5.0 GHz. Additionally, send and receive circuitry 630 includes circuitry, such as Ethernet circuitry 640 for sending and receiving data (for example, smart floor tile data) over a wired connection. In some embodiments, send and receive circuitry 630 further comprises circuitry for sending and receiving data using other wired or wireless communication protocols, such as Bluetooth Low Energy or Zigbee circuitry.

Additionally, according to certain embodiments, send and receive circuitry 630 includes a network interface 650, which operates to interconnect master control device 600 with one or more networks. Network interface 650 may, depending on embodiments, have a network address expressed as a node ID, a port number or an IP address. According to certain embodiments, network interface 650 is implemented as hardware, such as by a network interface card (NIC). Alternatively, network interface 650 may be implemented as software, such as by an instance of the java.net.NetworkInterface class. Additionally, according to some embodiments, network interface 650 supports communications over multiple protocols, such as TCP/IP as well as wireless protocols, such as 3G or Bluetooth.

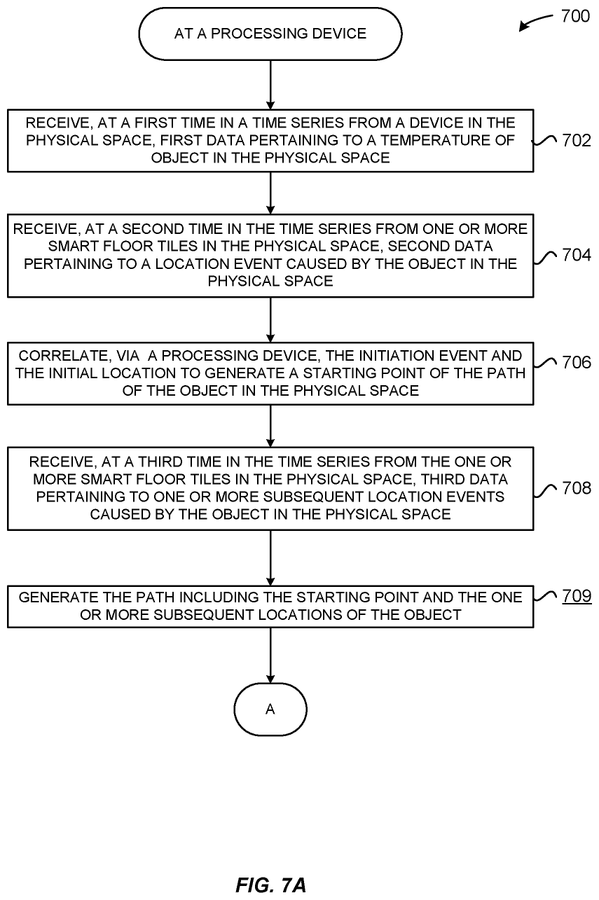

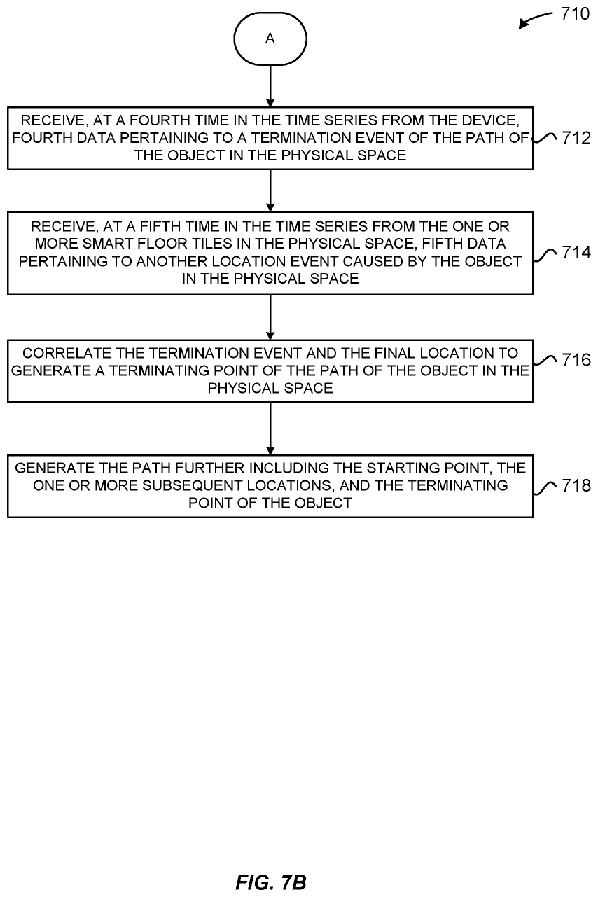

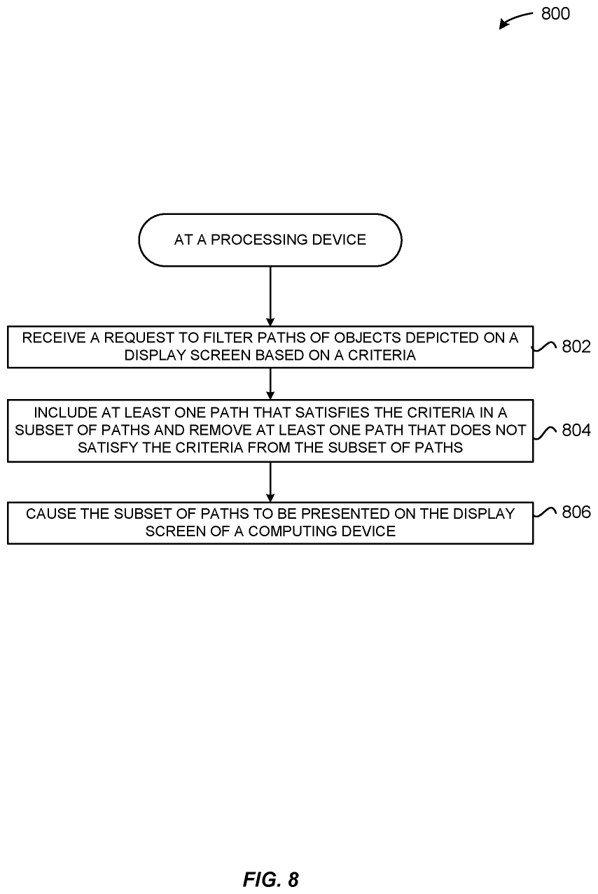

FIG. 7A illustrate an example of a method 700 for generating a path of a person in a physical space using smart floor tiles 112 according to certain embodiments of this disclosure. The method 700 may be performed by processing logic that may include hardware (circuitry, dedicated logic, etc.), software, or a combination of both. The method 700 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 700. The method 700 may be implemented as computer instructions stored on a memory device and executable by the one or more processors. In certain implementations, the method 700 may be performed by a single processing thread. Alternatively, the method 700 may be performed by two or more processing threads, each thread implementing one or more individual functions, routines, subroutines, or operations of the methods.

At block 702, the processing device may receive, at a first time in a time series, from a device (e.g., camera 50, reader device, etc.) in a physical space (first room 21), first data pertaining to an initiation event of the path of the object (e.g., person 25) in the physical space. The first data may include an identity of the person, employment position of the person in an entity, a job title of the person, an entity identity that employs the person, a gender of the person, an age of the person, a timestamp of the data, a temperature of the person, and the like. The initiation event may correspond to the person checking in for an event being held in the physical space. In some embodiments, when the device is a camera 50, the processing device may perform facial recognition techniques using facial image data received from the camera 50 to determine an identity of the person. The processing device may obtain information pertaining to the person based on the identity of the person. The information may include an entity for which the person works, an employment position of the person within the entity, or some combination thereof.

At block 704, the processing device may receive, at a second time in the time series from one or more smart floor tiles 112 in the physical space, second data pertaining to a location event caused by the object in the physical space. The location event may include an initial location of the object in the physical space. The initial location may be generated by one or more detected forces at the one or more smart floor tiles 112. The second data may be impression tile data received when the person steps onto a first smart floor tile 112 in the physical space. In some embodiments, the person may be standing on the first smart floor tile 112 when the initiation event occurs. That is, the initiation event and the location event may occur contemporaneously at substantially the same time in the time series. In some embodiments, the first time and the second time may differ less than a threshold period of time, or the first time and the second time may be substantially the same. The location event may include data pertaining to the one or more smart tiles 112 the object pressed, such as an identifier of the one or more smart floor tiles 112, a timestamp of when the one or more smart floor tiles 112 changed from an idle state to an active state, a duration of being in the active state, and the like.

At block 706, the processing device may correlate the initiation event and the initial location to generate a starting point of a path of the object in the physical space. In some embodiments, the starting point may be overlaid on a virtual representation of the physical space and the path of the object may be generated and presented in real-time or near real-time as the object moves around the physical space.

At block 708, the processing device may receive, at a third time in the time series from the one or more smart floor tiles 112 in the physical space, third data pertaining to one or more subsequent location events caused by the object in the physical space. The one or more subsequent location events may include one or more subsequent locations of the object in the physical space. The one or more subsequent location events may include data pertaining to the one or more smart tiles 112 the object pressed, such as an identifier of the one or more smart floor tiles 112, a timestamp of when the one or more smart floor tiles 112 changed from an idle state to an active state, a duration of being in the active state, and the like.

At block 709, the processing device may generate the path of the object including the starting point and the one or more subsequent locations of the object.

FIG. 7B illustrates an example of a method 710 continued from FIG. 7A according to certain embodiments of this disclosure. The method 710 may be performed by processing logic that may include hardware (circuitry, dedicated logic, etc.), software, or a combination of both. The method 710 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 710. The method 710 may be implemented as computer instructions stored on a memory device and executable by the one or more processors. In certain implementations, the method 710 may be performed by a single processing thread. Alternatively, the method 710 may be performed by two or more processing threads, each thread implementing one or more individual functions, routines, subroutines, or operations of the methods.

At block 712, the processing device may receive, at a fourth time in the time series from a device (e.g., camera 50, reader, etc.), fourth data pertaining to a termination event of the path of the object in the physical space.

At block 714, the processing device may receive, at a fifth time in the time series from the one or more smart floor tiles 112 in the physical space, fifth data pertaining to another location event caused by the object in the physical space. The another location event may correspond to when the user leaves the physical space (e.g., by checking out with a badge or any electronic device). The another location event may include a final location of the object in the physical space. The another location event may include data pertaining to the one or more smart tiles 112 the object pressed, such as an identifier of the one or more smart floor tiles 112, a timestamp of when the one or more smart floor tiles 112 changed from an idle state to an active state, a duration of being in the active state, and the like.

At block 716, the processing device may correlate the termination event and the final location to generate a terminating point of the path of the object in the physical space.

At block 718, the processing device may generate the path using the starting point, the one or more subsequent locations, and the terminating point of the object. Block 718 may result in the full path of the object in the physical space. The full path may be presented on a user interface of a computing device.

In some embodiments, the processing device may generate a second path for a second person in the physical space. The processing device may generate an overlay image by overlaying the path of the first person with the second path of the second object in a virtual representation of the physical space. The different paths may be represented using different or the same visual elements (e.g., color, boldness, etc.). The processing device may cause the overlay image to be presented on a computing device.

FIG. 8 illustrates an example of a method 800 for filtering paths of objects presented on a display screen according to certain embodiments of this disclosure. The method 800 may be performed by processing logic that may include hardware (circuitry, dedicated logic, etc.), software, or a combination of both. The method 800 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 800. The method 800 may be implemented as computer instructions stored on a memory device and executable by the one or more processors. In certain implementations, the method 800 may be performed by a single processing thread. Alternatively, the method 800 may be performed by two or more processing threads, each thread implementing one or more individual functions, routines, subroutines, or operations of the methods.

At block 802, the processing device may receive a request to filter paths of objects depicted on a user interface of a display screen based on a criteria. The criteria may be employment position, job title, entity identity for which people work, gender, age, or some combination thereof.

At block 804, the processing device may include at least one path that satisfies the criteria in a subset of paths and remove at least one path that does not satisfy the criteria from the subset of paths. For example, if the user selects to view paths of people having a manager position, the processing device may include the paths of all manager positions and remove other paths of people that do not have the manager position.

At block 806, the processing device may cause the subset of paths to be presented on the display screen of a computing device. The subset of paths may provide an improved user interface that increases the user's experience using the computing device because it includes only the desired paths of people in the physical area. Further, computing resources may be reduced by generating the subset of paths because fewer paths may be generated based on the criteria. Also less data may be transmitted over the network to the computing device displaying the subset because there are fewer paths in the subset based on the criteria.

FIG. 9 illustrates an example of a method 900 for presenting a longest path of an object in a physical space according to certain embodiments of this disclosure. The method 900 may be performed by processing logic that may include hardware (circuitry, dedicated logic, etc.), software, or a combination of both. The method 900 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 900. The method 900 may be implemented as computer instructions stored on a memory device and executable by the one or more processors. In certain implementations, the method 900 may be performed by a single processing thread. Alternatively, the method 900 may be performed by two or more processing threads, each thread implementing one or more individual functions, routines, subroutines, or operations of the methods.

At block 902, the processing device may receive a request to present a longest path of at least one object from the set of paths of the set of objects (e.g., people) based on a distance at least one object traveled, an amount of time the at least one object spent in the physical space, or some combination thereof.

At block 904, the processing device may determine one or more zones the at least one object attended in the longest path. The one or more zones may be determined using a virtual representation of the physical space and selecting the zones including smart floor tiles 112 through which the path of the at least one object traversed.

At block 906, the processing device may overlay the longest path of the at least one object on the one or more zones to generate a composite zone and path image.

At block 908, the processing device may cause the composite zone and path image to be presented on a display screen of the computing device. In some embodiments, the shortest path may also be selected and presented on the display screen. The longest path and the shortest path may be presented concurrently. In some embodiments, any suitable length of path in any combination may be selected and presented on a virtual representation of the physical space as desired.

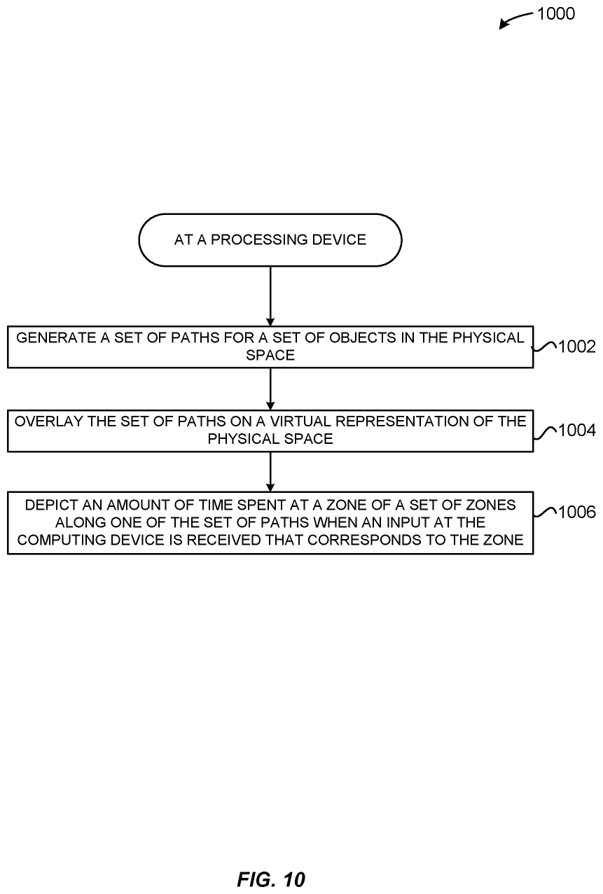

FIG. 10 illustrates an example of a method 1000 for presenting amount of times objects spent at certain zones in a physical space according to certain embodiments of this disclosure. The method 1000 may be performed by processing logic that may include hardware (circuitry, dedicated logic, etc.), software, or a combination of both. The method 1000 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1000. The method 1000 may be implemented as computer instructions stored on a memory device and executable by the one or more processors. In certain implementations, the method 1000 may be performed by a single processing thread. Alternatively, the method 1000 may be performed by two or more processing threads, each thread implementing one or more individual functions, routines, subroutines, or operations of the methods.

At block 1002, the processing device may generate a set of paths for a set of objects in the physical space. At block 1004, the processing device may overlay the set of paths on a virtual representation of the physical space.

At block 1006, the processing device may depict an amount of time spent at a zone of a set of zones along one of the set of paths when an input at the computing device is received that corresponds to the zone. In some embodiments, the user may select any point on the path of any person to determine the amount of time that person spent at a location at the selected point. Granular location and duration details may be provided using the data obtained via the smart floor tiles 112.

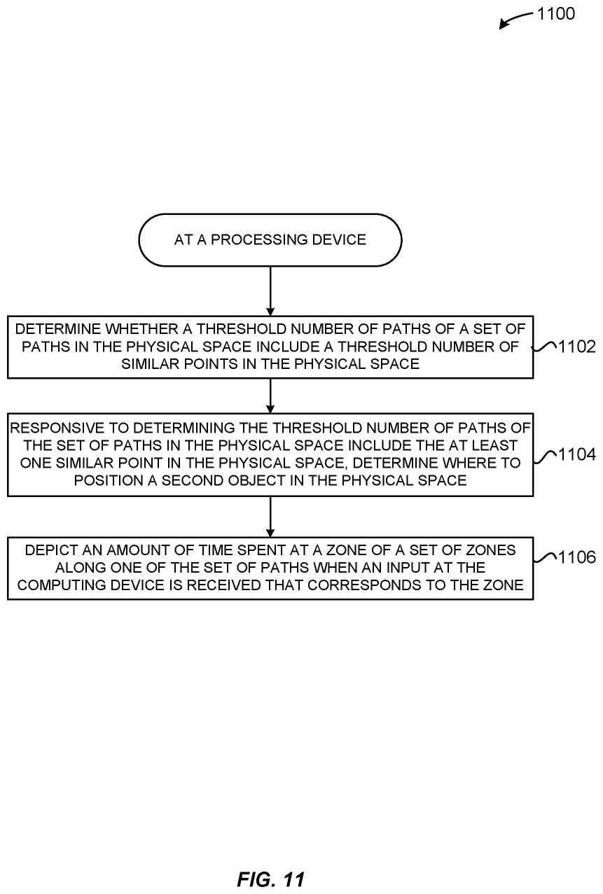

FIG. 11 illustrates an example of a method 1100 for determining where to place objects based on paths of people according to certain embodiments of this disclosure. The method 1100 may be performed by processing logic that may include hardware (circuitry, dedicated logic, etc.), software, or a combination of both. The method 1100 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1100. The method 1100 may be implemented as computer instructions stored on a memory device and executable by the one or more processors. In certain implementations, the method 1100 may be performed by a single processing thread. Alternatively, the method 1100 may be performed by two or more processing threads, each thread implementing one or more individual functions, routines, subroutines, or operations of the methods.

At block 1102, the processing device may determine whether a threshold number of paths of a set of paths in the physical space include a threshold number of similar points in the physical space. At block 1104, responsive to determining the threshold number of paths of the set of paths in the physical space include the at least one similar point in the physical space, the processing device may determine where to position a second object in the physical space. At block 1106, the processing device may depict an amount of time spent at a zone of a set of zones along one of the set of paths when an input at the computing device is received that corresponds to the zone, a person, a path, a booth, or the like.

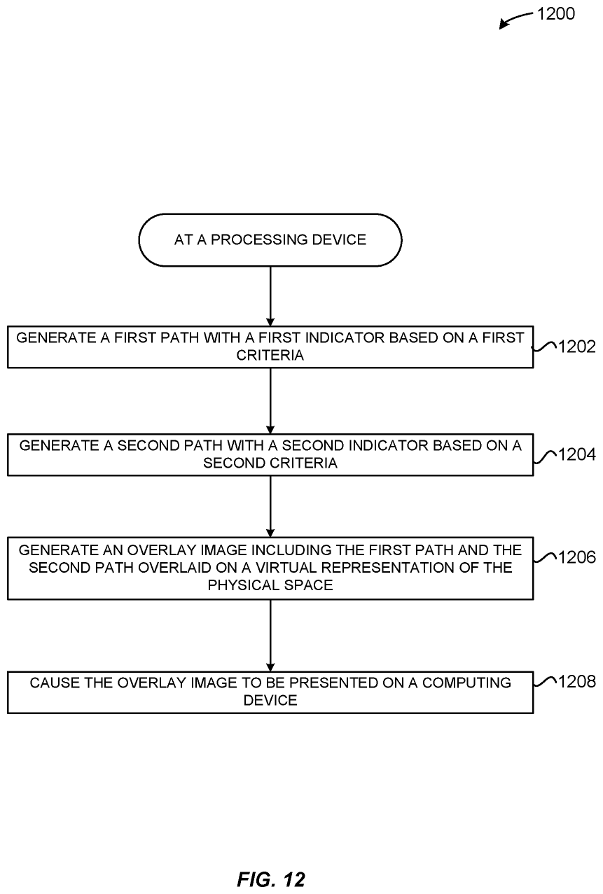

FIG. 12 illustrates an example of a method 1200 for overlaying paths of objects based on criteria according to certain embodiments of this disclosure. The method 1200 may be performed by processing logic that may include hardware (circuitry, dedicated logic, etc.), software, or a combination of both. The method 1200 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1200. The method 1200 may be implemented as computer instructions stored on a memory device and executable by the one or more processors. In certain implementations, the method 1200 may be performed by a single processing thread. Alternatively, the method 1200 may be performed by two or more processing threads, each thread implementing one or more individual functions, routines, subroutines, or operations of the methods.

At block 1202, the processing device may generate a first path with a first indicator based on a first criteria. The criteria may be job title, company name, age, gender, longest path, shortest path, etc. The first indicator may be a first color for the first path.

At block 1204, the processing device may generate a second path with a second indicator based on a second criteria. At block 1206, the processing device may generate an overlay image including the first path and the second path overlaid on a virtual representation of the physical space. At block 1208, the processing device may cause the overlay image to be presented on a computing device.

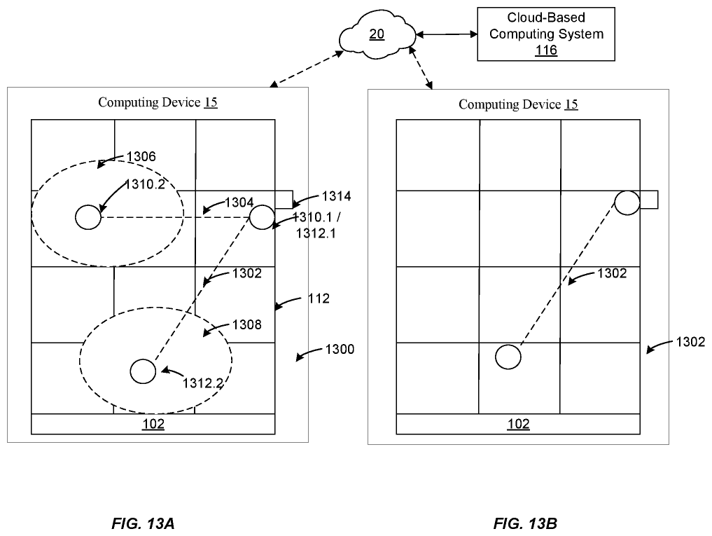

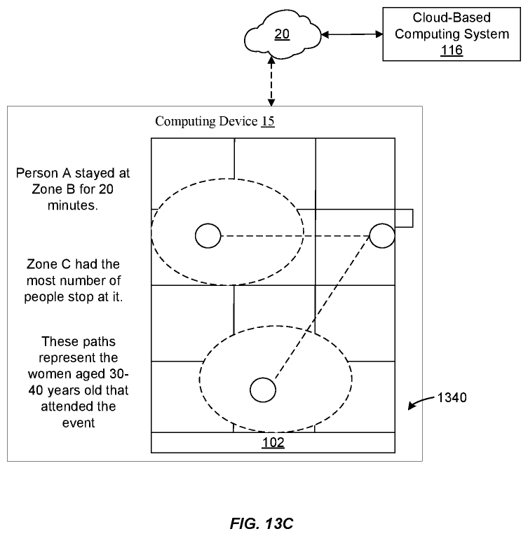

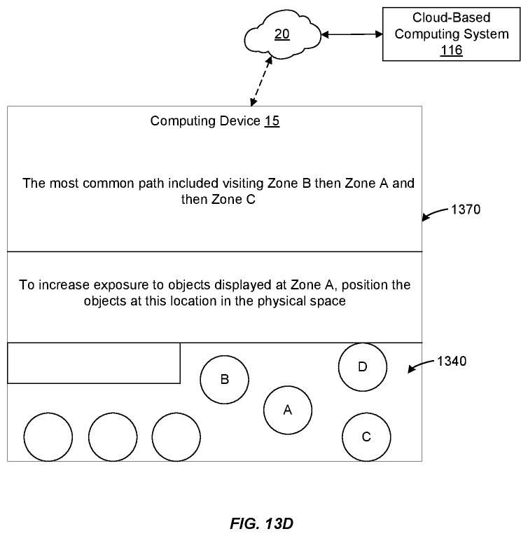

FIG. 13A illustrates an example user interface 1300 presenting paths 1300 and 1304 of people in a physical space according to certain embodiments of this disclosure. More particularly, the user interface 1300 presents a virtual representation of the first room 21, for example, from an above perspective. The user interface 1300 presents the smart floor tiles 112 and/or moulding section 102 that are arranged in the physical space. The user interface 1300 may include a visual representation mapping various zones 1306 and 1308 including various booths in the physical space.

An entrance to the physical space may include a device 1314 at which the user checks in for the event being held in the physical space. The device 1314 may be a reader device and/or a camera 50. The device 1314 may send data to the cloud-based computing system 116 to perform the methods disclosed herein.

For example, the data may be included in an initiation event that is used to generate a starting point of the path of the person. When the person enters the physical space, the person may press one or more first smart floor tiles 112 that transmit measurement data to the cloud-based computing system 116. The measurement data may be included in a location event and may include an initial location of the person in the physical space. The initial location and the initiation event may be used to generate the starting position of the path of the person. The measurement data obtained by the smart floor tiles 112 and sent to the cloud-based computing system 116 may be used during later location events and a termination location event to generate a full path of the person.

As depicted, two starting points 1310.1 and 1312.1 are overlaid on a smart floor tile 112 in the user interface 1300. Starting point 1310.1 is included as part of path 1304 and starting point 1312.1 is included as part of path 1302. Termination points 1310.2 and 1312.2. The termination point 1310.2 ends in zone 1306 and termination point 1312.2 ends in zone 1308. If the user places the cursor or selects any portion of the path (e.g., using a touchscreen), additional details of the paths 1304 and 1302 may be presented. For example, a duration of time the person spent at any of the points in the paths 1304 may be presented.

FIG. 13B illustrates an example user interface 1302 presenting a filtered path of a person in a physical space according to certain embodiments of this disclosure. In some embodiments, the paths presented in the user interface 1302 may be filtered based on any suitable criteria. For example, the user may select to view the paths of a person having a certain employment positon (e.g., a chief level position), and the user interface 1300 presents the path 1302 of the person having the certain employment position and removes the path 1304 of the person that does not have that employment position.

FIG. 13C illustrates an example user interface 1304 presenting information pertaining to paths of people in a physical space according to certain embodiments of this disclosure. As depicted, the user interface 1340 presents “Person A stayed at Zone B for 20 minutes”, “Zone C had the most number of people stop at it”, and “These paths represent the women aged 30-40 years old that attended the event.” As may be appreciated, the improve user interface 1304 may greatly enhance the experience of a user using the computing device 15 as the analytics enabled and disclosed herein may be very beneficial. Any suitable subset of paths may be generated using any suitable criteria.

FIG. 13D illustrates an example user interface 1370 presenting other information pertaining to a path of a person in a physical space and a recommendation where to place an object in the physical space based on path analytics according to certain embodiments of this disclosure. As depicted, the user interface 1370 presents “The most common path included visiting Zone B then Zone A and then Zone C”. The cloud-based computing system 116 may analyze the paths by comparing them to determine the most common path, the least common path, the durations spent at each zone, booth, or object in the physical space, and the like.

The user interface 1370 also presents “To increase exposure to objects displayed at Zone A, position the objects at this location in the physical space”. A visual representation 1372 presents the recommended location for objects in Zone A relative to other Zones B, C, and D. Accordingly, the cloud-based computing system 116 may determine the ideal locations for increasing traffic and/or attendance in zones and may recommend where to locate the zones, the booths in the zones, and/or the objects displayed at particular booths based on path analytics performed herein.

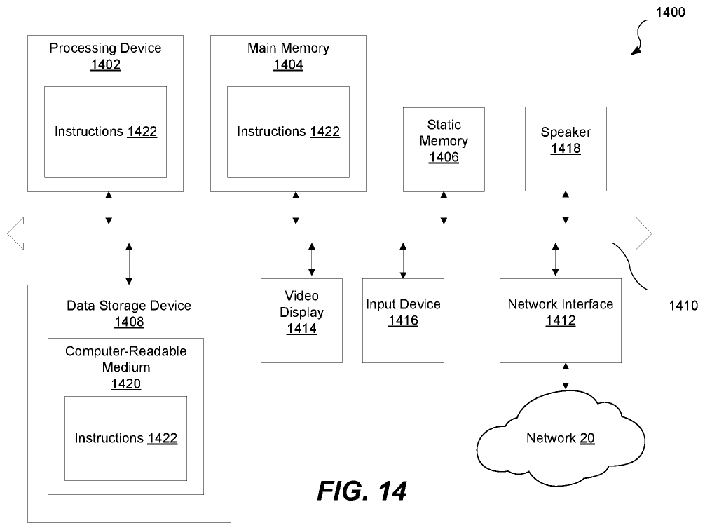

FIG. 14 illustrates an example computer system 1400, which can perform any one or more of the methods described herein. In one example, computer system 1400 may include one or more components that correspond to the computing device 12, the computing device 15, one or more servers 128 of the cloud-based computing system 116, the electronic device 13, the camera 50, the moulding section 102, the smart floor tile 112, or one or more training engines 152 of the cloud-based computing system 116 of FIG. 1B. The computer system 1400 may be connected (e.g., networked) to other computer systems in a LAN, an intranet, an extranet, or the Internet. The computer system 1400 may operate in the capacity of a server in a client-server network environment. The computer system 1400 may be a personal computer (PC), a tablet computer, a laptop, a wearable (e.g., wristband), a set-top box (STB), a personal Digital Assistant (PDA), a smartphone, a camera, a video camera, or any device capable of executing a set of instructions (sequential or otherwise) that specify actions to be taken by that device. Some or all of the components computer system 1400 may be included in the camera 50, the moulding section 102, and/or the smart floor tile 112. Further, while only a single computer system is illustrated, the term “computer” shall also be taken to include any collection of computers that individually or jointly execute a set (or multiple sets) of instructions to perform any one or more of the methods discussed herein.

The computer system 1400 includes a processing device 1402, a main memory 1404 (e.g., read-only memory (ROM), solid state drive (SSD), flash memory, dynamic random access memory (DRAM) such as synchronous DRAM (SDRAM)), a static memory 1406 (e.g., solid state drive (SSD), flash memory, static random access memory (SRAM)), and a data storage device 1408, which communicate with each other via a bus 1410.

Processing device 1402 represents one or more general-purpose processing devices such as a microprocessor, central processing unit, or the like. More particularly, the processing device 1402 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1402 may also be one or more special-purpose processing devices such as an application specific integrated circuit (ASIC), a field programmable gate array (FPGA), a digital signal processor (DSP), network processor, or the like. The processing device 1402 is configured to execute instructions for performing any of the operations and steps discussed herein.

The computer system 1400 may further include a network interface device 1412. The computer system 1400 also may include a video display 1414 (e.g., a liquid crystal display (LCD) or a cathode ray tube (CRT)), one or more input devices 1416 (e.g., a keyboard and/or a mouse), and one or more speakers 1418 (e.g., a speaker). In one illustrative example, the video display 1414 and the input device(s) 1416 may be combined into a single component or device (e.g., an LCD touch screen).

The data storage device 1416 may include a computer-readable medium 1420 on which the instructions 1422 embodying any one or more of the methodologies or functions described herein are stored. The instructions 1422 may also reside, completely or at least partially, within the main memory 1404 and/or within the processing device 1402 during execution thereof by the computer system 1400. As such, the main memory 1404 and the processing device 1402 also constitute computer-readable media. The instructions 1422 may further be transmitted or received over a network via the network interface device 1412.

While the computer-readable storage medium 1420 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

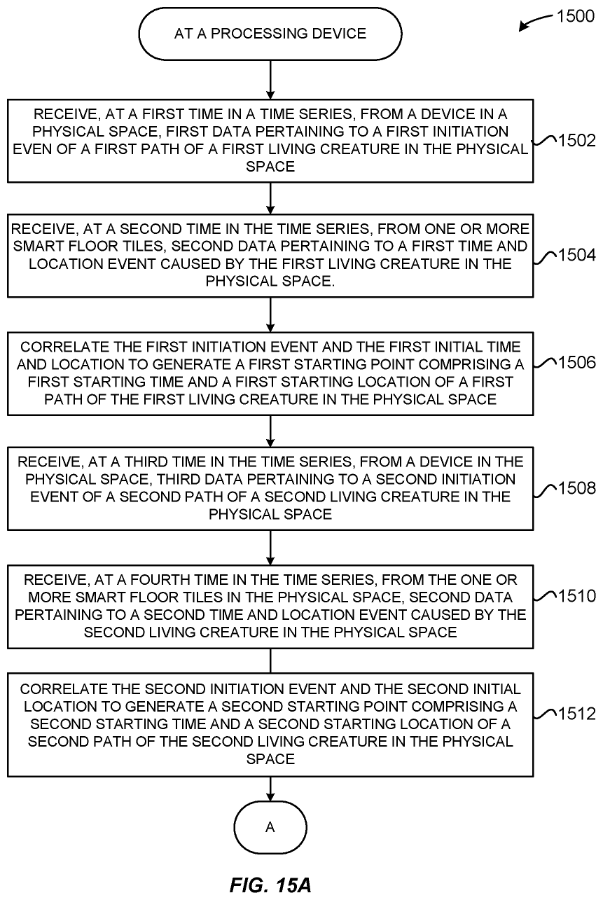

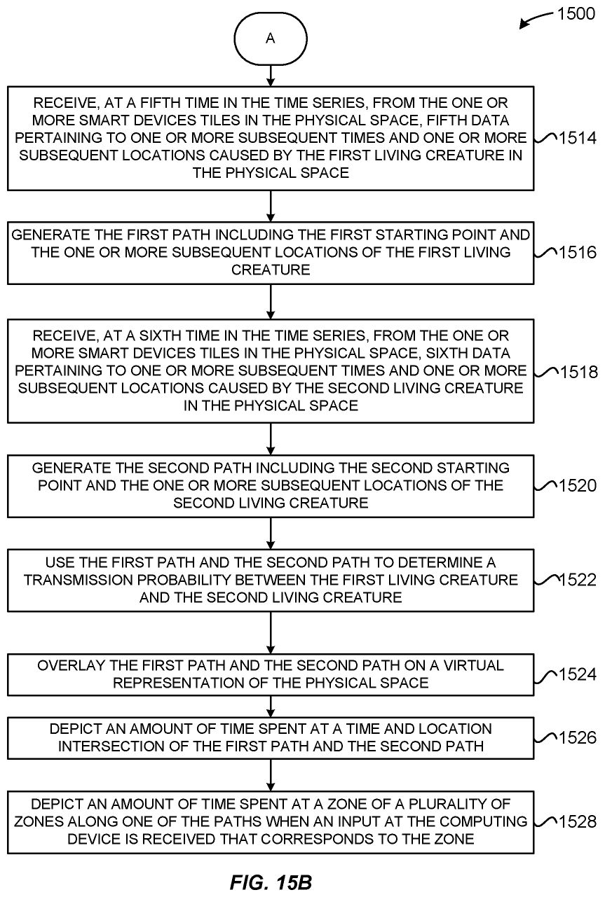

FIG. 15 illustrate an example of a method 1500 for tracking potential disease spread between living creatures within a physical space using smart floor tiles 112 according to certain embodiments of this disclosure. The method 700 may be performed by processing logic that may include hardware (circuitry, dedicated logic, etc.), software, or a combination of both. The method 700 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 700. The method 700 may be implemented as computer instructions stored on a memory device and executable by the one or more processors. In certain implementations, the method 700 may be performed by a single processing thread. Alternatively, the method 700 may be performed by two or more processing threads, each thread implementing one or more individual functions, routines, subroutines, or operations of the methods.

At block 1502, the processing device may receive, at a first time in the time series, from a device in the physical space (e.g., camera 50, reader device, thermal sensor 52, etc.), first data pertaining to a first initiation event of a first path of a first living creature (e.g., person 25) in the physical space. The first data may include a gender of the person, an age of the person, a disease risk factor of the person, whether the person is wearing a face mask, an identity of the person, an employment position of the person in an entity, the entity for which the person works, a timestamp of the data, and the like. The first initiation event may correspond to the person checking in to the physical space (i.e., signing in at the lobby). In some embodiments, when the device is a camera 50, the processing device may perform facial recognition techniques using facial image data received from the camera 50 to determine an identity of the person. In some embodiments, when the device is a thermal sensor 52, the processing device may compare a detected temperature of the person to a threshold value above which the person is considered to have an elevated likelihood of being infected by an infectious disease (e.g., COVID-19). The processing device may obtain information pertaining to the person based on the identity of the person. The information may include an entity for which the person works, an employment position of the person within the entity, a medical history of the person, or some combination thereof.

At block 1504, the processing device may receive, at a second time in the time series from one or more smart floor tiles (e.g., smart floor tiles 122) in the physical space, second data pertaining to a first time and location event caused by the first living creature in the physical space, wherein the first time and location event comprises a first initial location of the first living creature in the physical space. The first time and location event may include an initial location of the person in the physical space. The initial location may be generated by one or more detected forces at the one or more smart floor tiles 112. The second data may be impression tile data received when the person steps onto a first smart floor tile 112 in the physical space. In some embodiments, the person may be standing on the first smart floor tile 112 when the initiation event occurs. That is, the initiation event and the time and location event may occur contemporaneously at substantially the same time in the time series. In some embodiments, the first time and the second time may differ less than a threshold period of time, or the first time and the second time may be substantially the same. The time and location event may include data pertaining to the one or more smart tiles 112 the person pressed, such as an identifier of the one or more smart floor tiles 112, a timestamp of when the one or more smart floor tiles 112 changed from an idle state to an active state, a duration of being in the active state, and the like.

At block 1506, the processing device may correlate, the first initiation event and the first initial time and location to generate a first starting point comprising a first starting time and first starting location of a first path of the first living creature in the physical space. In some embodiments, the starting point may be overlaid on a virtual representation of the physical space and the path of the object may be generated and presented in real-time or near real-time as the object moves around the physical space.

At block 1508, the processing device may receive, at a third time in the time series, from a device in the physical space (e.g., smart floor tiles 112, moulding sections 102, camera 50, reader device, thermal sensor 52, etc.), third data pertaining to a second initiation event of a second path of a second living creature (e.g., another person 25) in the physical space. The third data may include a gender of the person, an age of the person, a disease risk factor of the person, whether the person is wearing a face mask, an identity of the person, an employment position of the person in an entity, the entity for which the person works, a timestamp of the data, and the like. The second initiation event may correspond to the person checking in to the physical space (i.e., signing in at the lobby). In some embodiments, when the device is a camera 50, the processing device may perform facial recognition techniques using facial image data received from the camera 50 to determine an identity of the person. In some embodiments, when the device is a thermal sensor 52, the processing device may compare a detected temperature of the person to a threshold value above which the person is considered to have an elevated likelihood of being infected by an infectious disease (e.g., COVID-19). The processing device may obtain information pertaining to the person based on the identity of the person. The information may include an entity for which the person works, an employment position of the person within the entity, a medical history of the person, or some combination thereof.

At block 1510, the processing device may receive, at a fourth time in the time series from one or more smart floor tiles (e.g., smart floor tiles 112) in the physical space, second data pertaining to a second time and location event caused by the second living creature in the physical space, wherein the second time and location event comprises a second initial location of the second living creature in the physical space. The second time and location event may include an initial location of the second living creature in the physical space. The initial location may be generated by one or more detected forces at the one or more smart floor tiles 112. The second data may be impression tile data received when the second person steps onto a first smart floor tile 112 in the physical space. In some embodiments, the second person may be standing on the first smart floor tile 112 when the initiation event occurs. That is, the initiation event and the time and location event may occur contemporaneously at substantially the same time in the time series. In some embodiments, the first time and the second time may differ less than a threshold period of time, or the first time and the second time may be substantially the same. The time and location event may include data pertaining to the one or more smart tiles 112 the person pressed, such as an identifier of the one or more smart floor tiles 112, a timestamp of when the one or more smart floor tiles 112 changed from an idle state to an active state, a duration of being in the active state, and the like.

At block 1512, the processing device may correlate the second initiation event and the second initial location to generate a second starting point comprising a second starting time and a second starting location of a first path of the second living creature in the physical space. In some embodiments, the starting point may be overlaid on a virtual representation of the physical space and the path of the second living creature may be generated and presented in real-time or near real-time as the second living creature moves around the physical space.

At block 1514, the processing device may receive, at a fifth time in the time series from the one or more smart devices tiles in the physical space, fifth data pertaining to one or more first subsequent time and location events caused by the first living creature in the physical space. The one or more first subsequent time and location events include one or more first subsequent times and one or more first subsequent locations of the first living creature in the physical space. The times and locations may be generated by one or more detected forces at the one or more smart floor tiles 112. The fifth data may be impression tile data received when the person steps onto another smart floor tile 112 in the physical space. The time and location event may include data pertaining to the one or more smart tiles 112 the person pressed, such as an identifier of the one or more smart floor tiles 112, a timestamp of when the one or more smart floor tiles 112 changed from an idle state to an active state, a duration of being in the active state, and the like.

At block 1516, the processing device may generate the first path including the starting point and the one or more subsequent locations of the first living creature.

At block 1518, the processing device may receive, at a sixth time in the time series from the one or more smart devices tiles in the physical space, sixth data pertaining to one or more second subsequent time and location events caused by the second living creature in the physical space. The one or more second subsequent time and location events include one or more second subsequent times and one or more second subsequent locations of the second living creature in the physical space. The times and locations may be generated by one or more detected forces at the one or more smart floor tiles 112. The sixth data may be impression tile data received when the second person steps onto another smart floor tile 112 in the physical space. The time and location event may include data pertaining to the one or more smart tiles 112 the second person pressed, such as an identifier of the one or more smart floor tiles 112, a timestamp of when the one or more smart floor tiles 112 changed from an idle state to an active state, a duration of being in the active state, and the like.

At block 1520, the processing device may generate the second path including the second starting point and the one or more subsequent locations of the second living creature.

At block 1522, the processing device may use the first path and the second path to determine a transmission probability between the first living creature and the second living creature. The transmission probability is the probability that, if the first living creature had a transmissible disease, the first living creature passed on that transmissible disease to the second living creature. For example, the processing device can calculate the transmission probability using how close the first living creature got to the second living creature (i.e., the distance between the first creature and the second creature, whether social distancing regulations or recommendations were followed), how much time the first living creature spent in proximity to the second living creature, whether the first living creature was wearing personal protective equipment (e.g., a mask), whether the second creature was wearing personal protective equipment. The transmission probability may be based solely on the closest distance between the first living creature and the second living creature. The transmission probability may be compared to a threshold transmission probability (i.e., a set probability that may correspond to desired actions to be taken, such as required testing or quarantining). Further, in some embodiments, the transmission probability may be based on the detected temperature of each of the first and second living creature.

If the transmission probability for a living creature is above a threshold amount, then a preventative action may be performed by the cloud-based computing system 116. The preventative action may include causing a user device 12 of the living creature to perform a function. That is, the cloud-based computing system 116 may distally control the user device 12 of the person in a physical space separate from where the server is located. The function performed by the user device 12 may include presenting a notification indicating the living create may be exposed to a certain disease or may have exposed someone else to the certain disease if the cloud-based computing system knows the person is already exposed to the certain disease. Further, the function may emit an alert (e.g., visually using a user interface, a light, a display screen; audibly using a speaker; using haptics via a haptic feature) that indicates that the transmission probability exceeds the threshold amount. The function may include presenting a notification that the living creature should be tested and to see a medical professional immediately or to initiate a telemedicine session with a medical professional. Another preventative action may include the cloud-based computing device controlling another electronic device in the physical space to perform a function (e.g., sound an alarm, emit an announcement of the threshold amount of the transmission probability being exceeded in that physical space, or the like). Further, another preventative action may include the cloud-based computing device controlling a user device 12 of a medical professional (e.g., a nurse) that is taking care of the person with the transmission probability exceeding the threshold amount. The cloud-based computing device may cause the user device 12 of the nurse to display a notification indicating the person may have transmitted or been exposed to the certain disease, to administer a test on the person, to take the vital signs of the person, or the like.

These probabilities may be accessed after the interaction in order to engage in contact tracing. For example, if the first living creature is later determined to be infected with an infectious disease (e.g., COVID-19), the probability that the first living creature infected the second living creature could be used in order to determine whether the second living creature should be quarantined or tested. This can be repeated for additional living creatures.

At block 1524, the processing device may overlay the paths on a virtual representation of the physical space. This may be used to help visualize the spread of infection or the extent to which social distancing restrictions are being followed.

At block 1526, the processing device may depict an amount of time spent at a time and location intersection of the paths. This amount of time may be used in visualizing how likely it was that transmission occurred.

At block 1528, the processing device may depict an amount of time spent at a zone of a plurality of zones along one of the paths when an input at the computing device is received that corresponds to the zone. This information, along with the amounts of time spent at each of the zones along other paths may allow visualization of hot spots and aid in changing the arrangement of the physical space to reduce the potential for spread of coronavirus.

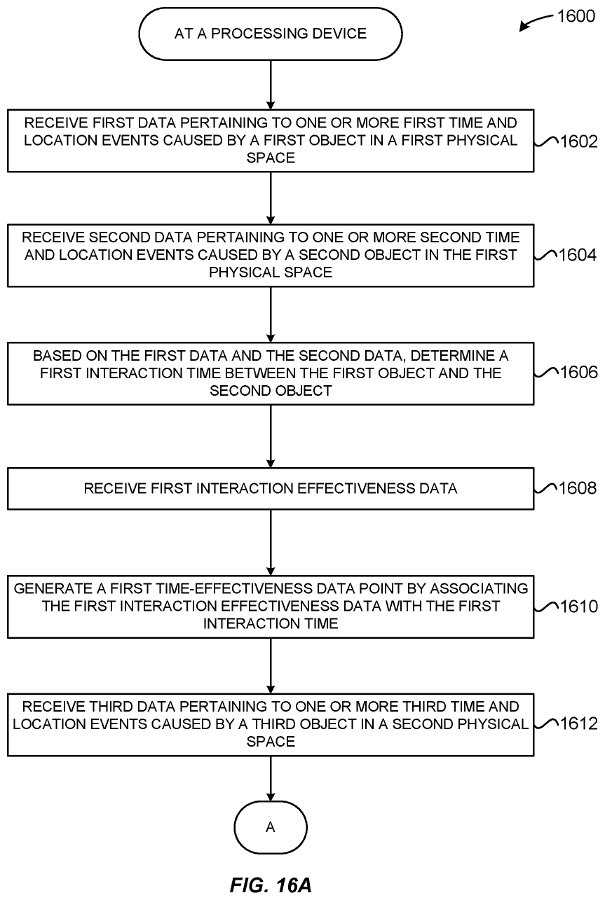

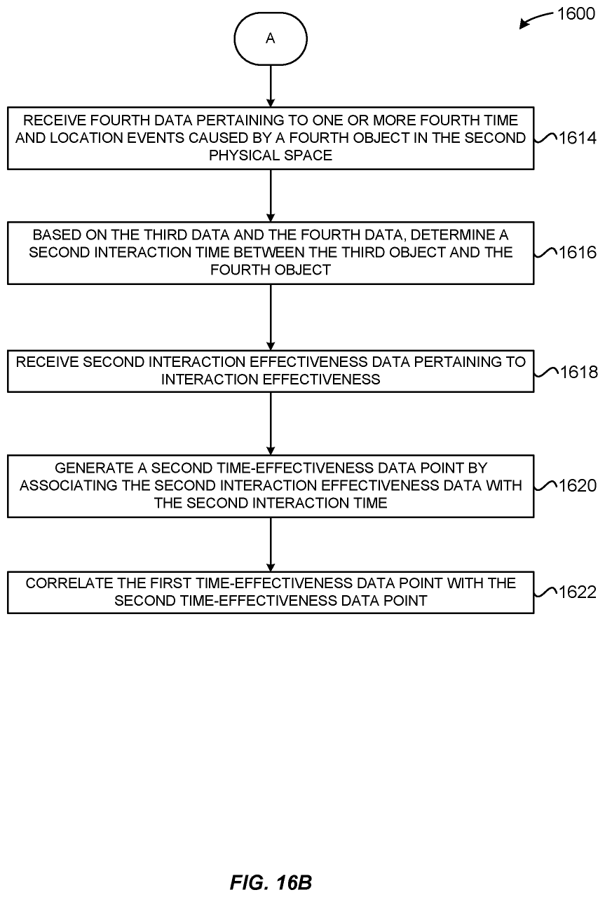

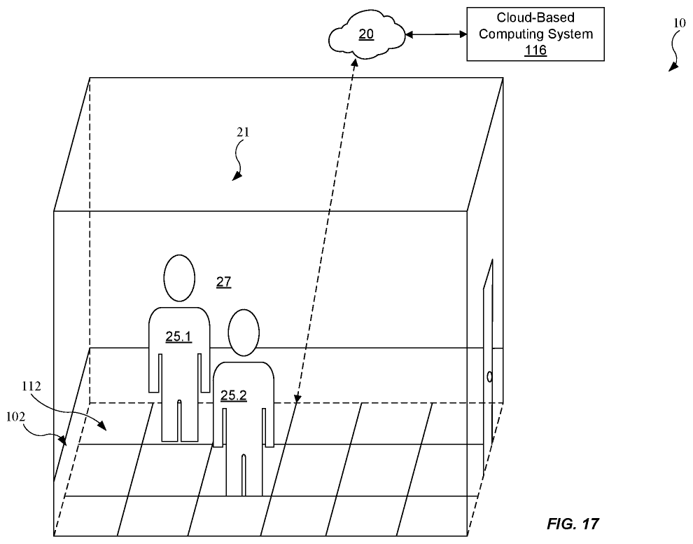

FIGS. 16A-B illustrate an example of a method 1600 for correlating interaction effectiveness to contact time within a physical space using smart floor tiles 112 according to certain embodiments of this disclosure. The method 1600 may be performed by processing logic that may include hardware (circuitry, dedicated logic, etc.), software, or a combination of both. The method 1600 and/or each of their individual functions, subroutines, or operations may be performed by one or more processors of a computing device (e.g., any component (server 128, training engine 152, machine learning models 154, etc.) of cloud-based computing system 116 of FIG. 1B) implementing the method 1600. The method 1600 may be implemented as computer instructions stored on a memory device and executable by the one or more processors. In certain implementations, the method 1600 may be performed by a single processing thread. Alternatively, the method 1600 may be performed by two or more processing threads, each thread implementing one or more individual functions, routines, subroutines, or operations of the methods.

At block 1602, the processing device may receive, from a first set of one or more smart floor tiles, first data pertaining to one or more first time and location events caused by a first object in a first physical space, wherein the one or more first time and location events include one or more first times and one or more first locations of the first object in the first physical space. In some embodiments, the first object is a patient undergoing treatment for a physical or psychological condition. In some embodiments, the patient is a human. In some embodiments, the patient is an animal. In some embodiments, the first physical space is a doctor's office, therapist's office, or a physical therapy center. In some embodiments, data may be associated with the first object, including a name associated with the object, a gender associated with the object, an identity of the object, an age associated with the object, a medical history associated with the object, one or more training programs undertaken by the object, an identity of the object, an employment position of the object in an entity, and the like. An example of the one or more first time and location events including one or more first times and one or more first locations of the first object in the first physical space received from the first set of one or more smart floor tiles includes time stamped pressure or presence location information. Specifically, a smart floor tile could send out information that, at a specific time, pressure has been applied to the smart floor tile. In some embodiments where there is furniture (e.g., a chair, table, couch, etc.) on the smart floor tile, the smart floor tile could send out information that pressure has increased on the smart floor tile, which could be used to determine that a person has placed their weight on the furniture.

At block 1604, the processing device may receive, from the first set of one or more smart floor tiles, second data pertaining to one or more second time and location events caused by a second object in the first physical space, wherein the one or more second time and location events include one or more second times and one or more second locations of the second object in the first physical space. In some embodiments, the second object is a practitioner (e.g., a doctor, a nurse, a psychotherapist, a physical therapist, a veterinarian, etc.). In some embodiments, data may be associated with the second object, including a name associated with the object, a gender associated with the object, an identity of the object, an age associated with the object, a medical history associated with the object, one or more training programs undertaken by the object, an identity of the object, an employment position of the object in an entity, and the like. An example of the one or more second time and location events including one or more second times and one or more second locations of the second object in the first physical space received from the first set of one or more smart floor tiles includes time stamped pressure or presence location information. Specifically, a smart floor tile could send out information that, at a specific time, pressure has been applied to the smart floor tile. In some embodiments where there is furniture (e.g., a chair, table, couch, etc.) on the smart floor tile, the smart floor tile could send out information that pressure has increased on the smart floor tile, which could be used to determine that an object has placed their weight on the furniture.

At block 1606, the processing device may, based on the first data and the second data, determine a first interaction time between the first object and the second object. The first interaction time may be determined by comparing the times and locations of the first object and the second object based on information provided by the smart floor tiles. The first interaction time may be determined based on the physical distance between the first object and the second object. The first interaction time may be determined based on the presence of the first object and the second object in the same room. The first interaction time may be based on the proximity of the first object and/or the second object to other objects. For instance, where a surgeon is performing remote surgery on a patient through remote-controlled surgical implements, the first interaction time may be based on the proximity of the surgeon to a set of controls for the remotely controlled surgical implements and the patient to the remotely controlled surgical implements. In some embodiments, wherein the first object is a patient and the second object is a practitioner, the first interaction time is a patient-to-practitioner contact time. As an example, the first interaction time may be determined to be thirty minutes.

At block 1608, the processing device may receive first interaction effectiveness data pertaining to a first interaction effectiveness. In some embodiments, the first interaction effectiveness is a first treatment effectiveness. The first treatment effectiveness may be received immediately after the treatment or at a later date when treatment effectiveness has been determined. The first treatment effectiveness may be based on patient health outcomes or specific treatment outcomes. The first interaction effectiveness may be based on a survey of the patient afterward. For instance, the first treatment effectiveness may be based on mental health screening questionnaires before and after psychological counseling. As an example, a mental health screening questionnaire before and after psychological counseling may show a first mental health improvement of five points on a given scale. The first interaction effectiveness may pertain to an increase or decrease in a property of the patient (e.g., increased strength, endurance, mobility, etc.; lower/higher blood pressure, temperature, heart rate, respiratory rate, etc.; lower/higher blood cell count, cognitive activity, etc.).

At block 1610, the processing device may generate a first time-effectiveness data point by associating the first interaction effectiveness data with the first interaction time. For example, the processing device may generate a first time-effectiveness data point indicating that a thirty minute counseling session resulted in a mental health improvement of five points on the given scale.

At block 1612, the processing device may receive, from a second set of one or more smart floor tiles, third data pertaining to one or more third time and location events caused by a third object in a second physical space, wherein the one or more third time and location events include one or more third times and one or more third locations of the third object in the second physical space. In some embodiments, the first object is a patient undergoing treatment for a physical or psychological condition. In some embodiments, the patient is a human. In some embodiments, the patient is an animal. In some embodiments, the second physical space is a doctor's office, therapist's office, or a physical therapy center. In some embodiments, data may be associated with the third object, including a name associated with the object, a gender associated with the object, an identity of the object, an age associated with the object, a medical history associated with the object, one or more training programs undertaken by the object, an identity of the object, an employment position of the object in an entity, and the like. An example of the one or more third time and location events including one or more third times and one or more third locations of the third object in the second physical space received from the second set of one or more smart floor tiles includes time stamped pressure or presence location information. Specifically, a smart floor tile could send out information that, at a specific time, pressure has been applied to the smart floor tile. In some embodiments where there is furniture (e.g., a chair, table, couch, etc.) on the smart floor tile, the smart floor tile could send out information that pressure has increased on the smart floor tile, which could be used to determine that a person has placed their weight on the furniture. In some embodiments, the third object is the same as the first object (e.g., the first object and the third object are the same patient). In some embodiments, the first physical space is the same as the second physical space (e.g., the first physical space and the second physical space are the same room of a doctor's office). In some embodiments, the second set of one or more smart floor tiles is the same as the first set of one or more smart floor tiles (e.g., the second set of one or more smart floor tiles and the first set of one or more smart floor tiles are the same set of smart floor tiles in the same room of a doctor's office).

At block 1614, the processing device may receive fourth data pertaining to one or more fourth time and location events caused by a fourth object in the second physical space, wherein the one or more fourth time and location events include one or more fourth times and one or more fourth locations of the fourth object in the second physical space. In some embodiments, the fourth object is a practitioner (e.g., a doctor, a nurse, a psychotherapist, a physical therapist, a veterinarian, etc.). In some embodiments, data may be associated with the fourth object, including a name associated with the object, a gender associated with the object, an identity of the object, an age associated with the object, a medical history associated with the object, one or more training programs undertaken by the object, an identity of the object, an employment position of the object in an entity, and the like. An example of the one or more fourth time and location events including one or more fourth times and one or more fourth locations of the fourth object in the second physical space received from the second set of one or more smart floor tiles includes time stamped pressure or presence location information. Specifically, a smart floor tile could send out information that, at a specific time, pressure has been applied to the smart floor tile. In some embodiments where there is furniture (e.g., a chair, table, couch, etc.) on the smart floor tile, the smart floor tile could send out information that pressure has increased on the smart floor tile, which could be used to determine that an object has placed their weight on the furniture. In some embodiments, the fourth object is the same as the second object (e.g., the fourth object and the second object are the same therapist).

At block 1616, the processing device may, based on the third data and the fourth data, determine a second interaction time between the third object and the fourth object. The second interaction time may be determined by comparing the times and locations of the third object and the fourth object, based on information provided by the smart floor tiles. The second interaction time may be determined based on the physical distance between the third object and the fourth object. The second interaction time may be determined based on the presence of the third object and the fourth object in the same room. The second interaction time may be based on the proximity of the third object and/or the fourth object to other objects. For instance, where a surgeon is performing remote surgery on a patient through remote-controlled surgical implements, the second interaction time may be based on the proximity of the surgeon to a set of controls for the remotely controlled surgical implements and the patient to the remotely controlled surgical implements. In some embodiments, wherein the third object is a patient and the fourth object is a practitioner, the second interaction time is a patient-to-practitioner contact time. As an example, the second interaction time may be determined to be sixty minutes.

At block 1618, the processing device may receive second interaction effectiveness data pertaining to a second interaction effectiveness. In some embodiments, the second interaction effectiveness is a second treatment effectiveness. The second treatment effectiveness may be received immediately after the treatment or at a later date when treatment effectiveness has been determined. The second treatment effectiveness may be based on patient health outcomes or specific treatment outcomes. The second interaction effectiveness may be based on a survey of the patient afterward. For instance, the second treatment effectiveness may be based on mental health screening questionnaires before and after psychological counseling. As an example, a mental health screening questionnaire before and after psychological counseling may show a second mental health improvement of eight points on the given scale. The second interaction effectiveness may pertain to an increase or decrease in a property of the patient (e.g., increased strength, endurance, mobility, etc.; lower/higher blood pressure, temperature, heart rate, respiratory rate, etc.; lower/higher blood cell count, cognitive activity, etc.).

At block 1620, the processing device may generate a second time-effectiveness data point by associating the second interaction effectiveness data with the second interaction time. For example, the processing device may generate a second time-effectiveness data point indicating that a sixty minute counseling session resulted in a mental health improvement of eight points on the given scale.

At block 1622, the processing device may correlate the first time-effectiveness data point with the second time-effectiveness data point. For example, the processing device may plot the first time-effectiveness data point and the second time-effectiveness data point on a graph. As another example, the processing device may determine based on the first time-effectiveness data point and the second time-effectiveness data point that the extra half hour associated with the second time-effectiveness data point resulted in another three points of mental health improvement on the given scale.

Any of the steps 1602-1622 may be performed or repeated in any suitable order to generate and correlate additional time-effectiveness data points (e.g., a third time-effectiveness data point, a fourth time-effectiveness data point, etc.) using the same objects or additional objects (e.g., a fifth object, a sixth object, etc.) in the first physical space, the second physical space, or additional physical spaces (e.g., a third physical space, a fourth physical space, etc.), with the first set of one or more smart floor tiles, the second set of one or more smart floor tiles, or additional sets of one or more smart floor tiles (e.g., a third set of one or more smart floor tiles, a fourth set of one or more smart floor tiles, etc.).

FIG. 17 shows an example of a physical space (i.e., the first physical space and/or the second physical space), in which the method 1600 can be applied. A room 21, in this example, is a physical space in which a first person 25.1 and a second person 25.2 are interacting. The room 21 may be any suitable room that includes a floor capable of being equipped with smart floor tiles 112 and/or moulding sections 102.

A cloud-based computing system 116 may receive first data from a first set of smart floor tiles 112 via a network 20 that indicates where and when the first person 25.1 steps and second data from the set of smart floor tiles 112 that indicates where and when the second person 25.2 steps. The data from the set of smart floor tiles 112 may include times and locations event that includes points in time and space where the first person 25.1 and the second person 25.2 are. The cloud-based computing device may determine an interaction time based on the proximity of the first person 25.1 and the second person 25.2 in time and space, as determined based on the data from the set of smart floor tiles. The room 21 may include any suitable features of the rooms 21, 23 described in FIGS. 1A and 1B.

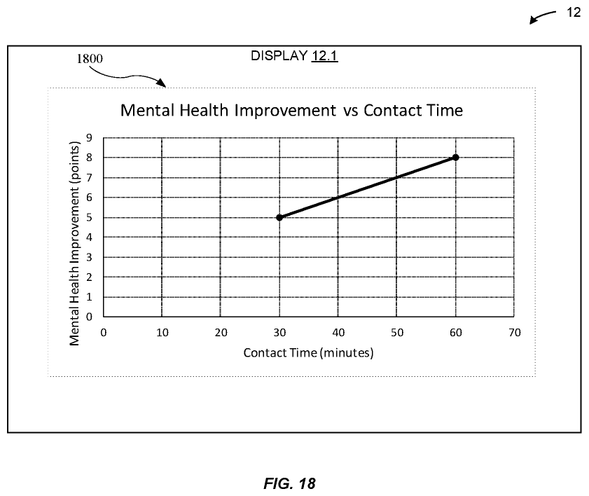

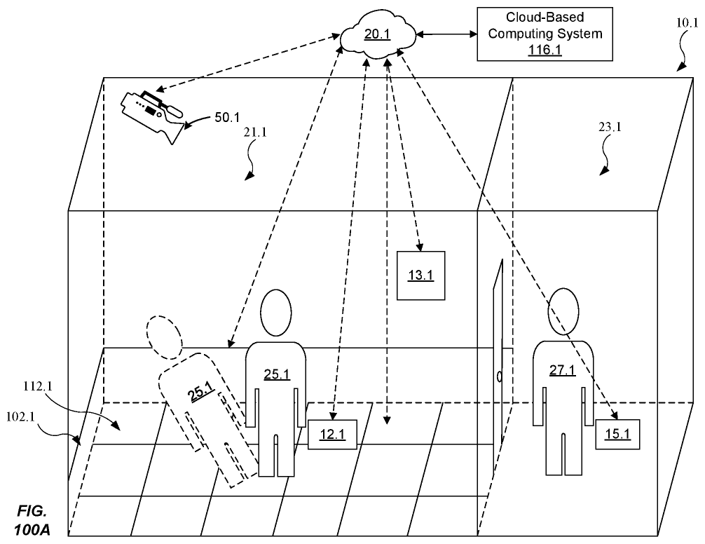

FIG. 18 shows an example of a graphical user interface 1800 that may be output on a display 12.1 of a user device 12 showing the correlation generated at block 1622 during performance of the method 1600.

### Environment Control Using Moulding Sections

The devices in the moulding sections may be independently controllable to control the environment temperature of a room. For example, the cloud-based computing system may cause the operating states of the devices to change based on the presence of the person in the room and/or based on the proximity of the person to certain moulding sections. In other words, a subset of the devices in moulding sections may be operated in an active operating state when the person is near those subset of devices, while another subset of devices in other moulding sections are operated in an inactive operating state. The devices may be independently controlled to provide desired temperatures for particular people that are present in the same room and based on the location of the people in the room. For example, user profiles may be stored and a first person may prefer to be cooler than a second person. If both people are in the same room, first devices of first moulding sections may be activated when the first person is near the first devices, and second devices of second moulding sections may be inactivated when the second person is near the second devices. Accordingly, the disclosed techniques may enable accurately, granularly, and/or efficiently operating of the devices to control the environment. Additional benefits, may include improving the user experience and comfort of living in a room implementing the disclosed embodiments.

The camera may provide a livestream of video data and/or image data to the cloud-based computing system. The data from the camera may be used to identify certain people in a room and/or track the path of the people in the room. Further, the data may be used to monitor one or more parameters pertaining to a gait of the person to aid in controlling the environment.

The cloud-based computing system may monitor one or more parameters of the person based on the measured data from the smart floor tiles, the moulding sections, and/or the camera. The one or more parameters may be associated with the gait of the person and/or the balance of the person. There are numerous other parameters associated with the person that may be monitored, as described in further detail below.

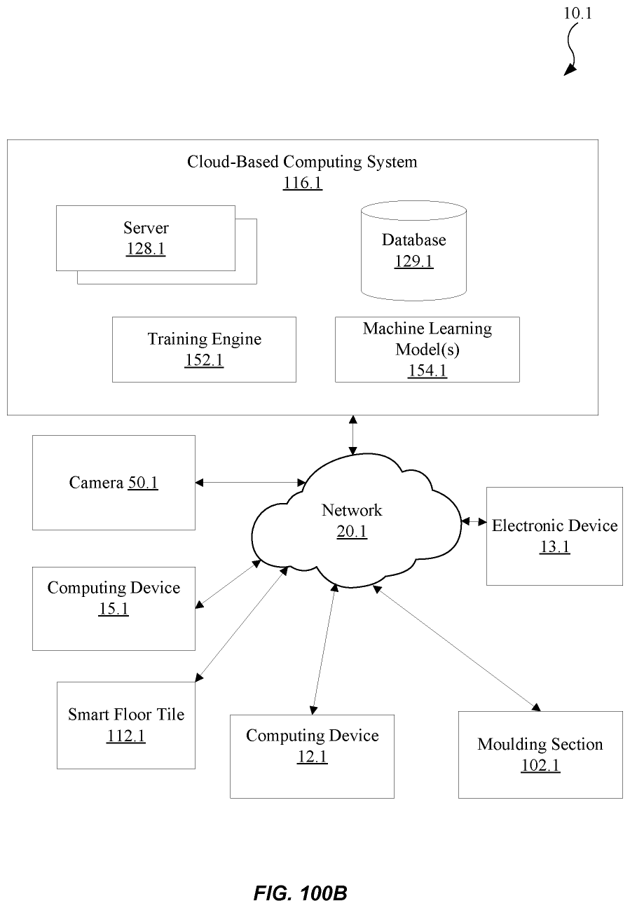

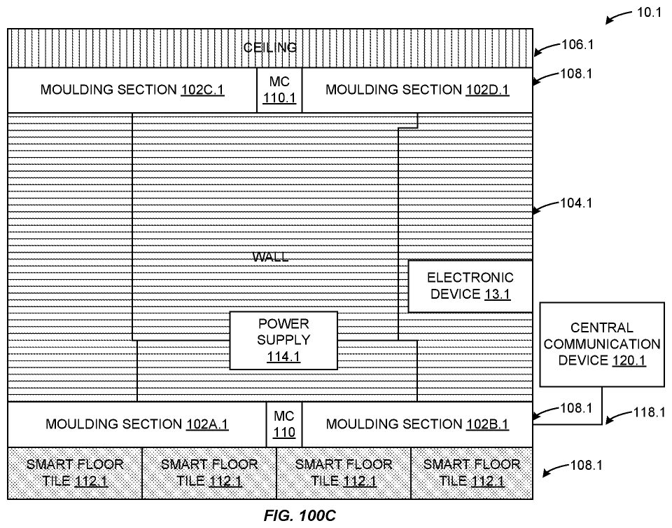

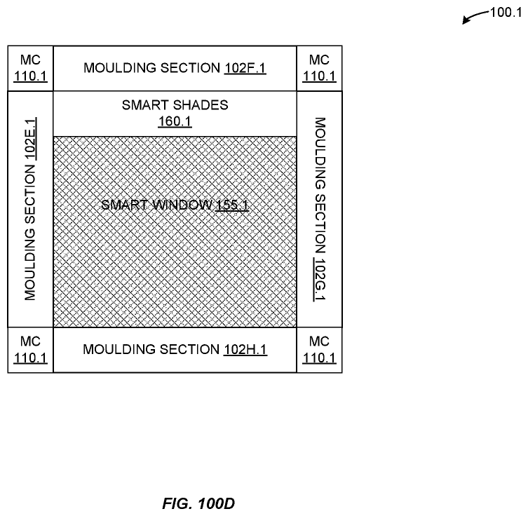

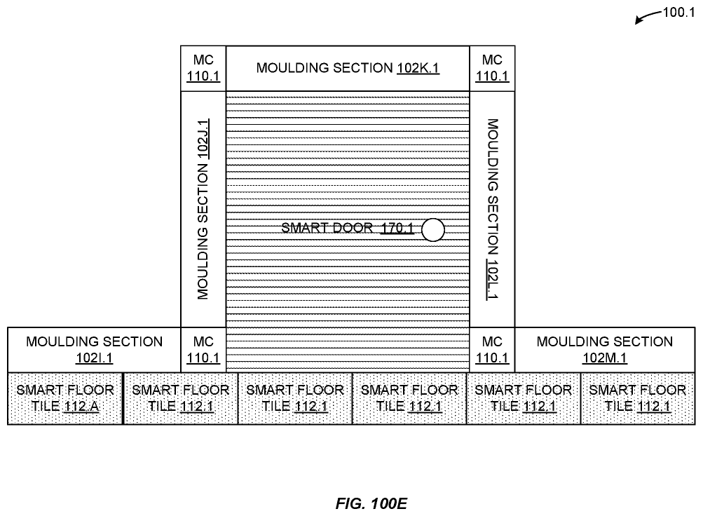

Turning now to the figures, FIGS. 100A-100E illustrate various example configurations of components of a system 10.1 according to certain embodiments of this disclosure. FIG. 100A visually depicts components of the system in a first room 21.1 and a second room 23.1 and FIG. 100B depicts a high-level component diagram of the system 10.1. For purposes of clarity, FIGS. 100A and 100B are discussed together below.

The first room 21.1, in this example, is a care room in a care facility where a person 25.1 is being treated. However, the first room 21.1 may be any suitable room that includes a floor capable of being equipped with smart floor tiles 112.1, moulding sections 102.1, and/or a camera 50.1. The second room 23.1, in this example, is a nursing station in the care facility.

The person 25.1 has a computing device 12.1, which may be a smartphone, a laptop, a tablet, a pager, or any suitable computing device. A medical personnel 27.1 in the second room 23.1 also has a computing device 15.1, which may be a smartphone, a laptop, a tablet, a pager, or any suitable computing device. The first room 21.1 may also include at least one electronic device 13.1, which may be any suitable electronic device, such as a smart thermostat, smart vacuum, smart light, smart speaker, smart electrical outlet, smart hub, smart appliance, smart television, etc.

Each of the smart floor tiles 112.1, moulding sections 102.1, camera 50.1, computing device 12.1, computing device 15.1, and/or electronic device 13.1 may be capable of communicating, either wirelessly and/or wired, with a cloud-based computing system 116.1 via a network 20.1. As used herein, a cloud-based computing system refers, without limitation, to any remote or distal computing system accessed over a network link. Each of the smart floor tiles 112.1, moulding sections 102.1, camera 50.1, computing device 12.1, computing device 15.1, and/or electronic device 13.1 may include one or more processing devices, memory devices, and/or network interface devices.

The network interface devices of the smart floor tiles 112.1, moulding sections 102.1, camera 50.1, computing device 12.1, computing device 15.1, and/or electronic device 13.1 may enable communication via a wireless protocol for transmitting data over short distances, such as Bluetooth, ZigBee, near field communication (NFC), etc. Additionally, the network interface devices may enable communicating data over long distances, and in one example, the smart floor tiles 112.1, moulding sections 102.1, camera 50.1, computing device 12.1, computing device 15.1, and/or electronic device 13.1 may communicate with the network 20.1. Network 20.1 may be a public network (e.g., connected to the Internet via wired (Ethernet) or wireless (WiFi)), a private network (e.g., a local area network (LAN), wide area network (WAN), virtual private network (VPN)), or a combination thereof.

The computing device 12.1 and/or computing device 15.1 may be any suitable computing device, such as a laptop, tablet, smartphone, or computer. The The computing device 12.1 and/or computing device 15.1 may include a display that is capable of presenting a user interface. The user interface may be implemented in computer instructions stored on a memory of the computing device 12.1 and/or computing device 15.1 and executed by a processing device of the computing device 12.1 and/or computing device 15.1. The user interface 105.1 be a stand-alone application that is installed on the computing device 12.1 and/or computing device 15.1 or may be an application (e.g., website) that executes via a web browser. The user interface may present various interventions including screens, notifications, and/or messages to the person 25.1 and/or the medical personnel 27.1.

For the computing device 12.1 of the person, the screens, notifications, and/or messages may be received from the cloud-based computing system 116.1 and may indicate that a fall event is predicted to occur in the future. The screens, notifications, and/or messages may encourage the person 25.1 to stop walking, to grab onto a supporting structure, to walk slower, or the like. The screens, notifications, and/or messages may enable the user to set a desired temperature for a particular room that may be used to control devices (e.g., fans) in the moulding sections 102.1 located in that particular room. For the computing device 15.1 of the medical personnel 27.1, the screens, notifications, and/or messages may be received from the cloud-based computing system 116.1 and may indicate that a fall event is predicted for the person 25.1. The screens, notifications, and/or messages may encourage the medical personnel 27.1 to tend to the person 25.1 in the first room 21.1 to attempt to prevent the fall event from occurring.

In some embodiments, the cloud-based computing system 116.1 may include one or more servers 128.1 that form a distributed, grid, and/or peer-to-peer (P2P) computing architecture. Each of the servers 128.1 may include one or more processing devices, memory devices, data storage, and/or network interface devices. The servers 128.1 may be in communication with one another via any suitable communication protocol. The servers 128.1 may receive data from the smart floor tiles 112.1, moulding sections 102.1, and/or the camera 50.1 and monitor a parameter pertaining to a gait of the person 25.1 based on the data. For example, the data may include pressure measurements obtained by a sensing device in the smart floor tile 112.1. The pressure measurements may be used to accurately track footsteps of the person 25.1, walking paths of the person 25.1, gait characteristics of the person 25.1, walking patterns of the person 25.1 throughout each day, and the like. The server 128.1 may track the path of the user and use the path to control the operating state of the devices included in the moulding sections 102.1, as described further herein.

In some embodiments, the cloud-based computing system 116.1 may include a training engine 152.1 and/or the one or more machine learning models 154.1. The training engine 152.1 and/or the one or more machine learning models 154.1 may be communicatively coupled to the servers 128.1 or may be included in one of the servers 128.1. In some embodiments, the training engine 152.1 and/or the machine learning models 154.1 may be included in the computing device 12.1, computing device 15.1, and/or electronic device 13.1.

The one or more of machine learning models 154.1 may refer to model artifacts created by the training engine 152.1 using training data that includes training inputs and corresponding target outputs (correct answers for respective training inputs). The training engine 152.1 may find patterns in the training data that map the training input to the target output (the answer to be predicted), and provide the machine learning models 154.1 that capture these patterns. The set of machine learning models 154.1 may comprise, e.g., a single level of linear or non-linear operations (e.g., a support vector machine [SVM]) or a deep network, i.e., a machine learning model comprising multiple levels of non-linear operations. Examples of such deep networks are neural networks including, without limitation, convolutional neural networks, recurrent neural networks with one or more hidden layers, and/or fully connected neural networks.

In some embodiments, the machine learning model 154.1 may be trained to determine which operating state(s) to operate a device(s) (e.g., fan) in the moulding sections 102.1. The machine learning model 154.1 may make the determination based on a user profile of preferred temperatures at certain times of the day, based on the current operating state of the device, based on the presence or absence of the user, based on the location of the user in relation to the moulding sections 102.1, and so forth.

In some embodiments, the cloud-based computing system 116 may include a database 129.1. The database 129.1 may store data pertaining to observations determined by the machine learning models 154.1. The observations may pertain to temperature preferences in a room at certain times of day for a user (e.g, a user profile), presence data of when the person 25.1 is present and absent from the room, and so forth. The training data used to train the machine learning models 154.1 may be stored in the database 129.1.

The camera 50.1 may be any suitable camera capable of obtaining data including video and/or images and transmitting the video and/or images to the cloud-based computing system 116.1 via the network 20.1. The data obtained by the camera 50.1 may include timestamps for the video and/or images. In some embodiments, the cloud-based computing system 116.1 may perform computer vision to extract high-dimensional digital data from the data received from the camera 50.1 and produce numerical or symbolic information. The numerical or symbolic information may represent the parameters monitored pertaining to the gait of the person 25.1 monitored by the cloud-based computing system 116.1.

As described further below, gait baseline parameters may be calibrated prior to the cloud-based computing system 116.1 determines whether a propensity for the fall event satisfies the threshold propensity condition. One or more tests may be performed to calibrate the gait baseline parameters. For example, a smart floor tile test may involve the person 25.1 walking across the first room 21.1 while the smart floor tiles 112.1 measure pressure of the person's footsteps and transmit data representing the measured data (e.g., amount of pressure, location of pressure, timestamp of measurement, etc.) to the cloud-based computing system 116.1. The cloud-based computing system may calibrate gait baseline parameters for the gait speed of the person 25.1, width between feet during gait of the person 25.1, stride length of the person 25.1, and the like. The gait baseline parameters may be subsequently used to compare with subsequent data pertaining to the gait of the person 25.1 to determine the amount of gait deterioration and/or the propensity for a fall event of the person 25.1.

As depicted in FIG. 100A, a fall event (represented by dashed user 25.1) may be predicted by the cloud-based computing system 116.1 based on the data received from the smart floor tile 112.1, moulding sections 102.1, and/or the camera 50.1. The cloud-based computing system 116.1 may select and perform various interventions to prevent the fall event.

FIGS. 100C-100E depict various example configurations of smart floor tiles 112.1, and/or moulding sections 102.1 according to certain embodiments of this disclosure. FIG. 100C depicts an example system 10.1 that is used in a physical space of a smart building (e.g., care facility). The depicted physical space includes a wall 104.1, a ceiling 106.1, and a floor 108.1 that define a room. Numerous moulding sections 102A.1, 102B.1, 102C.1, and 102D.1 are disposed in the physical space. For example, moulding sections 102A.1 and 102B.1 may form a baseboard or shoe moulding that is secured to the wall 108.1 and/or the floor 108.1. Moulding sections 102C.1 and 102D.1 may for a crown moulding that is secured to the wall 108.1 and/or the ceiling 106.1. Each moulding section 102A.1 may have different shapes and/or sizes.

The moulding sections 102.1 may each include various components, such as electrical conductors, sensors, processors, memories, network interfaces, and so forth. The electrical conductors may be partially or wholly enclosed within one or more of the moulding sections. For example, one electrical conductor may be a communication cable that is partially enclosed within the moulding section and exposed externally to the moulding section to electrically couple with another electrical conductor in the wall 108.1. In some embodiments, the electrical conductor may be communicably connected to at least one smart floor tile 112.1. In some embodiments, the electrical conductor may be in electrical communication with a power supply 114.1. In some embodiments, the power supply 114.1 may provide electrical power that is in the form of mains electricity general-purpose alternating current. In some embodiments, the power supply 114.1 may be a battery, a generator, or the like.

In some embodiments, the electrical conductor is configured for wired data transmission. To that end, in some embodiments the electrical conductor may be communicably coupled via cable 118.1 to a central communication device 120.1 (e.g., a hub, a modem, a router, etc.). Central communication device 120.1 may create a network, such as a wide area network, a local area network, or the like. Other electronic devices 13.1 may be in wired and/or wireless communication with the central communication device 120.1. Accordingly, the moulding section 102.1 may transmit data to the central communication device 120.1 to transmit to the electronic devices 13.1. The data may be control instructions that cause, for example, an the electronic device 13.1 to change a property based on a prediction that the person 25.1 is going to experience a fall event. In some embodiments, the moulding section 102A.1 may be in wired and/or wireless communication connection with the electronic device 13.1 without the use of the central communication device 120.1 via a network interface and/or cable. The electronic device 13.1 may be any suitable electronic device capable of changing an operational parameter in response to a control instruction.

In some embodiments, the electrical conductor may include an insulated electrical wiring assembly. In some embodiments, the electrical conductor may include a communications cable assembly. The moulding sections 102.1 may include a flame-retardant backing layer. The moulding sections 102.1 may be constructed using one or more materials selected from: wood, vinyl, rubber, fiberboard, and wood composite materials.

The moulding sections may be connected via one or more moulding connectors 110.1. A moulding connector 110.1 may enhance electrical conductivity between two moulding sections 102.1 by maintaining the conductivity between the electrical conductors of the two moulding sections 102.1. For example, the moulding connector 110.1 may include contacts and its own electrical conductor that forms a closed circuit when the two moulding sections are connected with the moulding connector 110.1. In some embodiments, the moulding connectors 110.1 may include a fiber optic relay to enhance the transfer of data between the moulding sections 102.1. It should be appreciated that the moulding sections 102.1 are modular and may be cut into any desired size to fit the dimensions of a perimeter of a physical space. The various sized portions of the moulding sections 102.1 may be connected with the moulding connectors 110.1 to maintain conductivity.

Moulding sections 102.1 may utilize a variety of sensing technologies, such as proximity sensors, optical sensors, membrane switches, pressure sensors, and/or capacitive sensors, to identify instances of an object proximate or located near the sensors in the moulding sections and to obtain data pertaining to a gait of the person 25.1. Proximity sensors may emit an electromagnetic field or a beam of electromagnetic radiation (infrared, for instance), and identify changes in the field or return signal. The object being sensed may be any suitable object, such as a human, an animal, a robot, furniture, appliances, and the like. Sensing devices in the moulding section may generate moulding section sensor data indicative of gait characteristics of the person 25.1, location (presence) of the person 25.1, the timestamp associated with the location of the person 25.1, and so forth.

The moulding section sensor data may be used to control one or more devices (e.g., fans) included in each of the moulding sections. The fans may be installed in the moulding sections such that air or wind generated by the fans is allowed to exit the moulding section (e.g., via a vent) and to change a temperature of the environment in which the moulding section is located.

The moulding section sensor data may be used alone or in combination with tile impression data generated by the smart floor tiles 112.1 and/or image data generated by the camera 50.1 to perform predict fall events for the person 25.1 and perform appropriate interventions to prevent the fall event from occuring. For example, the moulding section sensor data may be used to determine a control instruction to generate and to transmit to an electric device 13.1 and/or the smart floor tile 102A.1. The control instruction may include changing an operational parameter of the electronic device 13.1 based on the moulding section sensor data indicating the person 25.1 is going to experience a fall event. The control instruction may include instructing the smart floor tile 112.1 to reset one or more components based on an indication in the moulding section sensor data that the one or more components is malfunctioning and/or producing faulty results. Further, the moulding sections 102.1 may include a directional indicator (e.g., light) that is emits different colors of light, intensities of light, patterns of light, etc. based on a fall event being predicted by the cloud-based computing system 116.1.

In some embodiments, the moulding section sensor data can be used to verify the impression tile data and/or image data of the camera 50.1 is accurate for predicting a fall event for the person 25.1. Such a technique may improve accuracy of the determination. Further, if the moulding section sensor data, the impression tile data, and/or the image data do not align (e.g., the moulding section sensor data does not indicate a fall event will occur and the impression tile data indicates a fall event will occur), then further analysis may be performed. For example, tests can be performed to determine if there are defective sensors at the corresponding smart floor tile 112.1 and/or the corresponding moulding section 102.1 that generated the data. Further, control actions may be performed such as resetting one or more components of the moulding section 102.1 and/or the smart floor tile 112.1. In some embodiments, preference to certain data may be made by the cloud-based computing system 116.1. For example, in one embodiment, preference for the impression tile data may be made over the moulding section sensor data and/or the image data, such that if the impression tile data differs from the moudling section sensor data and/or the image data, the impression tile data is used to predict the propensity for the fall event.

FIG. 100D illustrates another configuration of the moulding sections 102.1. In this example, the moulding sections 102E.1-102H.1 surround a border of a smart window 155.1. The moulding sections 102.1 are connected via the moulding connector 110.1. As may be appreciated, the modular nature of the moulding sections 102.1 with the moulding connectors 110.1 enables forming a square around the window. Other shapes may be formed using the moulding sections 102.1 and the moulding connectors 110.1.

The moulding sections 102.1 may be electrically and/or communicably connected to the smart window 155.1 via electrical conductors and/or interfaces. The moulding sections 102.1 may provide power to the smart window 155.1, receive data from the smart window 155.1, and/or transmit data to the smart window 155.1. One example smart window includes the ability to change light properties using voltage that may be provided by the moulding sections 102.1. The moulding sections 102.1 may provide the voltage to control the amount of light let into a room based on predicting a propensity for a fall event. For example, if the moulding section sensor data, impression tile data, and/or image data indicates the person 25.1 has a high propensity for experiencing a fall event, the cloud-based computing system 116.1 may perform an intervention by causing the moulding sections 102.1 to instruct the smart window 155.1 to change a light property to allow light into the room. In some instances the cloud-based computing system 116.1 may communicate directly with the smart window 155.1 (e.g., electronic device 13.1).

In some embodiments, the moulding sections 102.1 may use sensors to detect when the smart window 155.1 is opened. The moulding sections 102.1 may determine whether the smart window 155.1 opening is performed at an expected time (e.g., when a home owner is at home) or at an unexpected time (e.g., when the home owner is away from home). The moulding sections 102.1, the camera 50.1, and/or the smart floor tile 112.1 may sense the occupancy patterns of certain objects (e.g., people) in the space in which the moulding sections 102.1 are disposed to determine a schedule of the objects. The schedule may be referenced when determining if an undesired opening (e.g., break-in event) occurs and the moulding sections 102.1 may be communicatively to an alarm system to trigger the alarm when the certain event occurs.

The schedule may also be referenced when determining a medical condition of the person 25.1. For example, if the schedule indicates that the person 25.1 went to the bathroom a certain number of times (e.g., 10) within a certain time period (e.g., 1 hour), the cloud-based computing system 116.1 may determine that the person has a urinary tract infection (UTI) and may perform an intervention, such as transmitting a message to the computing device 12.1 of the person 25.1. The message may indicate the potential UTI and recommend that the person 25.1 schedules an appointment with a medical personnel.

As depicted, at least moulding section 102F.1 is electrically and/or communicably coupled to smart shades 160.1. Again, the cloud-based computing system 116.1 may cause the moulding section 102F.1 to control the smart shades 160.1 to extend or retract to control the amount of light let into a room. In some embodiments, the cloud-based computing system 116.1 may communicate directly with the smart shades 160.1.

FIG. 100E illustrates another configuration of the moulding sections 102.1 and smart floor tiles 112.1. In this example, the moulding sections 102E.1-102H.1 surround a majority of a border of a smart door 170.1. The moulding sections 10211, 102K.1, and 102L.1 and/or the smart floor tile 112.1 may be electrically and/or communicably connected to the smart door 170.1 via electrical conductors and/or interfaces. The moulding sections 102.1 and/or smart floor tiles 112.1 may provide power to the smart door 170.1, receive data from the smart door 170.1, and/or transmit data to the smart door 170.1. In some embodiments, the moulding sections 102.1 and/or smart floor tiles 112.1 may control operation of the smart door 170.1. For example, if the moulding section sensor data and/or impression tile data indicates that no one is present in a house for a certain period of time, the moulding sections 102.1 and/or smart floor tiles 112.1 may determine a locked state of the smart door 170.1 and generate and transmit a control instruction to the smart door 170.1 to lock the smart door 170.1 if the smart door 170.1 is in an unlocked state.

In another example, the moulding section sensor data, impression tile data, and/or the image data may be used to generate gait profiles for people in a smart building (e.g., care facility). When a certain person is in the room near the smart door 170.1, the cloud-based computing device 116.1 may detect that person's presence based on the data received from the smart floor tiles, moulding sections 102.1, and/or camera 50.1. In some embodiments, if the person 25.1 is detected near the smart door 170.1, the cloud-based computing system 116.1 may determine whether the person 25.1 has a particular medical condition (e.g., alzheimers) and/or a flag is set that the person should not be allowed to leave the smart building. If the person is detected near the smart door 170.1 and the person 25.1 has the particular medical condition and/or the flag set, then the cloud-based computing system 116.1 may cause the moulding sections 102.1 and/or smart floor tiles 112.1 to control the smart door 170.1 to lock the smart door 170.1. In some embodiments, the cloud-based computing system 116.1 may communicate directly with the smart door 170.1 to cause the smart door 170.1 to lock.

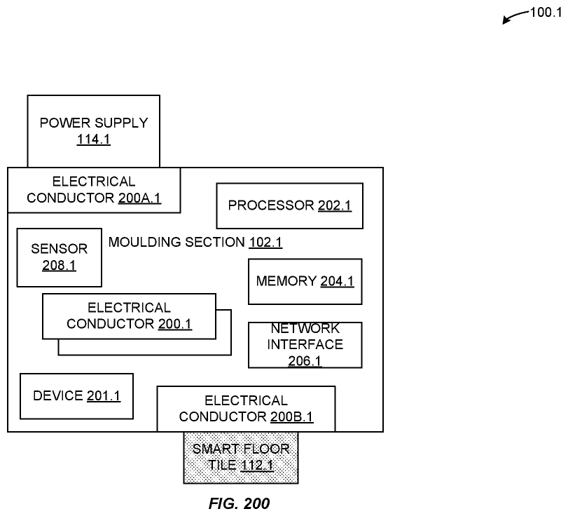

FIG. 200 illustrates an example component diagram of a moulding section 102.1 according to certain embodiments of this disclosure. As depicted, the moulding section 102.1 includes numerous electrical conductors 200.1, a device 201.1, a processor 202.1, a memory 204.1, a network interface 206.1, and a sensor 208.1. More or fewer components may be included in the moulding section 102.1. The electrical conductors may be insulated electrical wiring assemblies, communications cable assemblies, power supply assemblies, and so forth. As depicted, one electrical conductor 200A.1 may be in electrical communication with the power supply 114.1, and another electrical conductor 200B.1 may be communicably connected to at least one smart floor tile 112.1.

In various embodiments, the moulding section 102.1 further comprises a processor 202.1. In the non-limiting example shown in FIG. 200, processor 202.1 is a low-energy microcontroller, such as the ATMEGA328P by Atmel Corporation. According to other embodiments, processor 202.1 is the processor provided in other processing platforms, such as the processors provided by tablets, notebook or server computers.

In some embodiments, the device 201.1 may include any suitable fan. The device 201.1 may be electrically and/or communicatively coupled to the processor 202.1. The processor 202.1 may receive instructions from the cloud-based computing system 116.1 that causes the processor 202.1 to change an operating state of the device 201.1. The operating state may include active for producing air or wind, or inactive. The operating state may also include a mode type, such as heating, cooling, or venting, etc.

In the non-limiting example shown in FIG. 200, the moulding section 102.1 includes a memory 204.1. According to certain embodiments, memory 204.1 is a non-transitory memory containing program code to implement, for example, generation and transmission of control instructions, networking functionality, the algorithms for generating and analyzing locations, presence, and/or tracks, and the algorithms for determining gait deterioration and/or propensity for a fall event as described herein.

Additionally, according to certain embodiments, the moulding section 102.1 includes the network interface 206.1, which supports communication between the moulding section 102.1 and other devices in a network context in which smart building control using directional occupancy sensing and fall prediction/prevention is being implemented according to embodiments of this disclosure. In the non-limiting example shown in FIG. 200, network interface 206.1 includes circuitry for sending and receiving data using Wi-Fi, including, without limitation at 900 MHz, 2.8 GHz and 5.0 GHz. Additionally, network interface 206.1 includes circuitry, such as Ethernet circuitry for sending and receiving data (for example, smart floor tile data) over a wired connection. In some embodiments, network interface 206.1 further comprises circuitry for sending and receiving data using other wired or wireless communication protocols, such as Bluetooth Low Energy or Zigbee circuitry. The network interface 206.1 may enable communicating with the cloud-based computing device 116.1 via the network 20.1.

Additionally, according to certain embodiments, network interface 206.1 which operates to interconnect the moulding device 102.1 with one or more networks. Network interface 206.1 may, depending on embodiments, have a network address expressed as a node ID, a port number or an IP address. According to certain embodiments, network interface 206.1 is implemented as hardware, such as by a network interface card (NIC). Alternatively, network interface 206.1 may be implemented as software, such as by an instance of the java.net.NetworkInterface class. Additionally, according to some embodiments, network interface 206.1 supports communications over multiple protocols, such as TCP/IP as well as wireless protocols, such as 3G or Bluetooth. Network interface 206.1 may be in communication with the cloud-based computing system 116.1 of FIG. 100A.

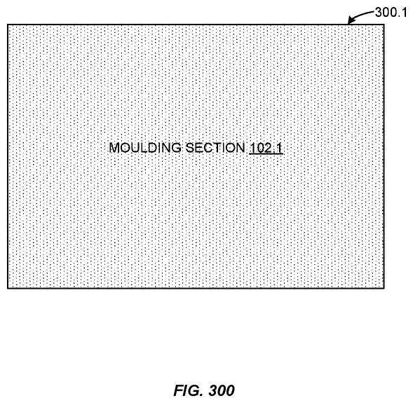

FIG. 300 illustrates an example backside view 300.1 of a moulding section 102.1 according to certain embodiments of this disclosure. As depicted by the dots 300.1, the backside of the moulding section 102.1 may include a fire-retardant backing layer positioned between the moulding section 102.1 and the wall to which the moulding section 102.1 is secured.

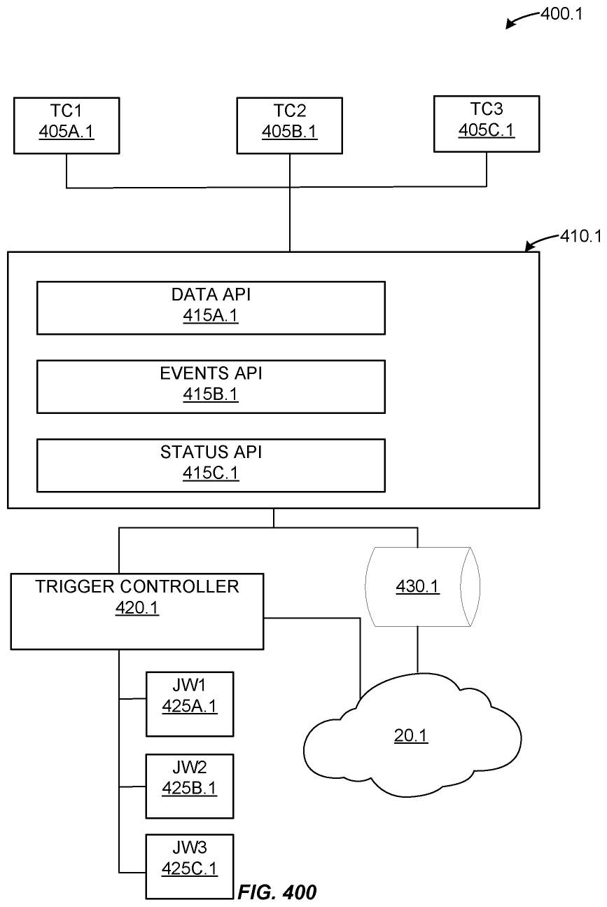

FIG. 400 illustrates a network and processing context 400.1 for smart building control using directional occupancy sensing and fall prediction/prevention according to certain embodiments of this disclosure. The embodiment of the network context 400.1 shown in FIG. 400 is for illustration only and other embodiments could be used without departing from the scope of the present disclosure.

In the non-limiting example shown in FIG. 400, a network context 400.1 includes one or more tile controllers 405A.1, 405B.1 and 405C.1, an API suite 410.1, a trigger controller 420.1, job workers 425A.1-425C.1, a database 430.1 and a network 435.1.

According to certain embodiments, each of tile controllers 405A.1-405C.1 is connected to a smart floor tile 112.1 in a physical space. Tile controllers 405A.1-405C.1 generate floor contact data (also referred to as impression tile data herein) from smart floor tiles in a physical space and transmit the generated floor contact data to API suite 410.1. In some embodiments, data from tile controllers 405A.1-405C.1 is provided to API suite 410.1 as a continuous stream. In the non-limiting example shown in FIG. 400, tile controllers 405A.1-405C.1 provide the generated floor contact data from the smart floor tile to API suite 410.1 via the internet. Other embodiments, wherein tile controllers 405A.1-405C.1 employ other mechanisms, such as a bus or Ethernet connection to provide the generated floor data to API suite 410.1 are possible and within the intended scope of this disclosure.

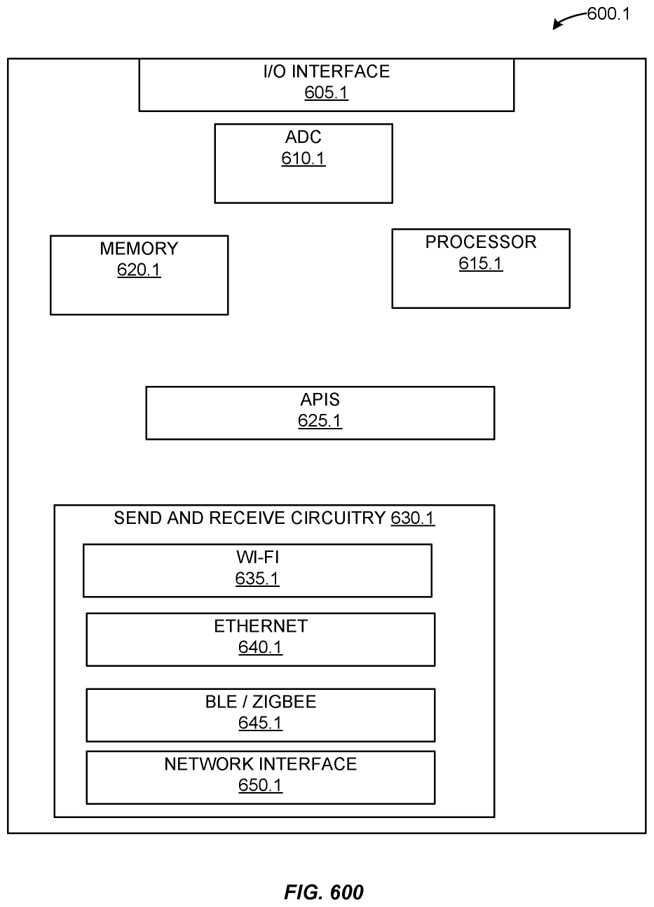

According to some embodiments, API suite 410.1 is embodied on a server 128.1 in the cloud-based computing system 116.1 connected via the internet to each of tile controllers 405A.1-405C.1. According to some embodiments, API suite is embodied on a master control device, such as master control device 600.1 shown in FIG. 600 of this disclosure. In the non-limiting example shown in FIG. 400, API suite 410.1 comprises a Data Application Programming Interface (API) 415A.1, an Events API 415B.1 and a Status API 215C.1.

In some embodiments, Data API 415A.1 is an API for receiving and recording tile data from each of tile controllers 405A.1-405C.1. Tile events include, for example, raw, or minimally processed data from the tile controllers, such as the time and data a particular smart floor tile was pressed and the duration of the period during which the smart floor tile was pressed. According to certain embodiments, Data API 415A.1 stores the received tile events in a database such as database 430.1. In the non-limiting example shown in FIG. 400, some or all of the tile events are received by API suite 410.1 as a stream of event data from tile controllers 405A.1-405C.1, Data API 415A.1 operates in conjunction with trigger controller 420.1 to generate and pass along triggers breaking the stream of tile event data into discrete portions for further analysis.

According to various embodiments, Events API 415B.1 receives data from tile controllers 405A.1-405C.1 and generates lower-level records of instantaneous contacts where a sensor of the smart floor tile is pressed and released.

In the non-limiting example shown in FIG. 400, Status API 415C.1 receives data from each of tile controllers 405A.1-405C.1 and generates records of the operational health (for example, CPU and memory usage, processor temperature, whether all of the sensors from which a tile controller receives inputs is operational) of each of tile controllers 405A.1-405C.1. According to certain embodiment, status API 415C.1 stores the generated records of the tile controllers' operational health in database 430.1.

According to some embodiments, trigger controller 420.1 operates to orchestrate the processing and analysis of data received from tile controllers 405A.1-405C.1. In addition to working with data API 415A.1 to define and set boundaries in the data stream from tile controllers 405A.1-405C.1 to break the received data stream into tractably sized and logically defined “chunks” for processing, trigger controller 420.1 also sends triggers to job workers 425A.1-425C.1 to perform processing and analysis tasks. The triggers comprise identifiers uniquely identifying each data processing job to be assigned to a job worker. In the non-limiting example shown in FIG. 400, the identifiers comprise: 1.) a sensor identifier (or an identifier otherwise uniquely identifying the location of contact); 2.) a time boundary start identifying a time in which the smart floor tile went from an idle state (for example, an completely open circuit, or, in the case of certain resistive sensors, a baseline or quiescent current level) to an active state (a closed circuit, or a current greater than the baseline or quiescent level); and 3.) a time boundary end defining the time in which a smart floor tile returned to the idle state.

In some embodiments, each of job workers 425A.1-425C.1 corresponds to an instance of a process performed at a computing platform, (for example, cloud-based computing system 116.1 in FIG. 100A) for determining tracks and performing an analysis of the tracks (e.g., such as predicting a propensity for a fall event and performing an intervention based on the propensity). Instances of processes may be added or subtracted depending on the number of events or possible events received by API suite 410.1 as part of the data stream from tile controllers 405A.1-205C.1. According to certain embodiments, job workers 425A.1-425C.1 perform an analysis of the data received from tile controllers 405A.1-405C.1, the analysis having, in some embodiments, two stages. A first stage comprises deriving footsteps, and paths, or tracks, from impression tile data. A second stage comprises characterizing those footsteps, and paths, or tracks, to determine gait characteristics of the person 25.1. The gait characteristics may be presented to an online dashboard (in some embodiments, provided by a UI on an electronic device, such as computing device 12.1 or 15.1 in FIG. 100A) and to generate control signals for devices (e.g., the computing devices 12.1 and/or 15.1, the electronic device 15.1, the moulding sections 102.1, the camera 50.1, and/or the smart floor tile 112.1 in FIG. 100A) controlling operational parameters of a physical space where the smart floor impression tile data were recorded.

In the non-limiting example shown in FIG. 400, job workers 425A.1-425C.1 perform the constituent processes of a method for analyzing smart floor tile impression tile data and/or moulding section sensor data to generate paths, or tracks. In some embodiments, an identity of the the person 25.1 may be correlated with the paths or tracks. For example, if the person scanned an ID badge when entering the physical space, their path may be recorded when the person takes their first step on a smart floor tile and their path may be correlated with an identifier received from scanning the badge. In this way, the paths of various people may be recorded (e.g., in a convention hall). This may be beneficial if certain people have desirable job titles (e.g., chief executive officer (CEO), vice president, president, etc.) and/or work at desirable client entities. For example, in some embodiments, the path of a CEO may be tracked in during a convention to determine which booths the CEO stopped at and/or an amount of time the CEO spent at each booth. Such data may be used to determine where to place certain booths in the future. For example, if a booth was visited by a threshold number of people having a certain title for a certain period of time, a recommendation may be generated and presented that recommends relocating the booth to a location in the convention hall that is more easily accessible to foot traffic. Likewise, if it is determined that a booth has poor visitation frequency based on the paths, or tracks, of attendees at the convention, a recommendation may be generated to relocate the booth to another location that is more easily accessible to foot traffic. In some embodiments, the machine learning models 154.1 may be trained to determine the paths, or tracks, of the people having various job titles and working for desired client entities, analyze their paths (e.g., which location the people visited, how long the people visited those locations, etc.), and generate recommendations.

According to certain embodiments, the method comprises the operations of obtaining impression image data, impression tile data, and/or moulding section sensor data from database 430.1, cleaning the obtained image data, impression tile data, and/or moulding section sensor data and reconstructing paths using the cleaned data. In some embodiments, cleaning the data includes removing extraneous sensor data, removing gaps between image data, impression tile data, and/or moulding section sensor data caused by sensor noise, removing long image data, impression tile data, and/or moulding section sensor data caused by objects placed on smart floor tiles, by objects placed in front of moulding sections, by objects stationary in image data, by defective sensors, and sorting image data, impression tile data, and/or moulding section sensor data by start time to produce sorted image data, impression tile data, and/or moulding section sensor data. According to certain embodiments, job workers 425A.1-425C.1 perform processes for reconstructing paths by implementing algorithms that first cluster image data, impression tile data, and/or moulding section sensor data that overlap in time or are spatially adjacent. Next, the clustered data is searched, and pairs of image data, impression tile data, and/or moulding section sensor data that start or end within a few milliseconds of one another are combined into footsteps and/or locations of the object, which are then linked together to form footsteps and/or locations. Footsteps and/or locations are further analyzed and linked to create paths.

According to certain embodiments, database 430.1 provides a repository of raw and processed image data, smart floor tile impression tile data, and/or moulding section sensor data, as well as data relating to the health and status of each of tile controllers 405A.1-405C.1 and moulding sections 102.1. In the non-limiting example shown in FIG. 400, database 430.1 is embodied on a server machine communicatively connected to the computing platforms providing API suite 410.1, trigger controller 420.1, and upon which job workers 425A.1-425C.1 execute. According to some embodiments, database 430.1 is embodied on the cloud-based computing system 116.1 as the database 129.1.

In the non-limiting example shown in FIG. 400, the computing platforms providing trigger controller 420.1 and database 430.1 are communicatively connected to one or more network(s) 20.1. According to embodiments, network 20.1 comprises any network suitable for distributing impression tile data, image data, moulding section sensor data, determined paths, determined gait deterioration of a parameter, determine propensity for a fall event, and control signals (e.g., interventions) based on determined propensities for fall events, including, without limitation, the internet or a local network (for example, an intranet) of a smart building.

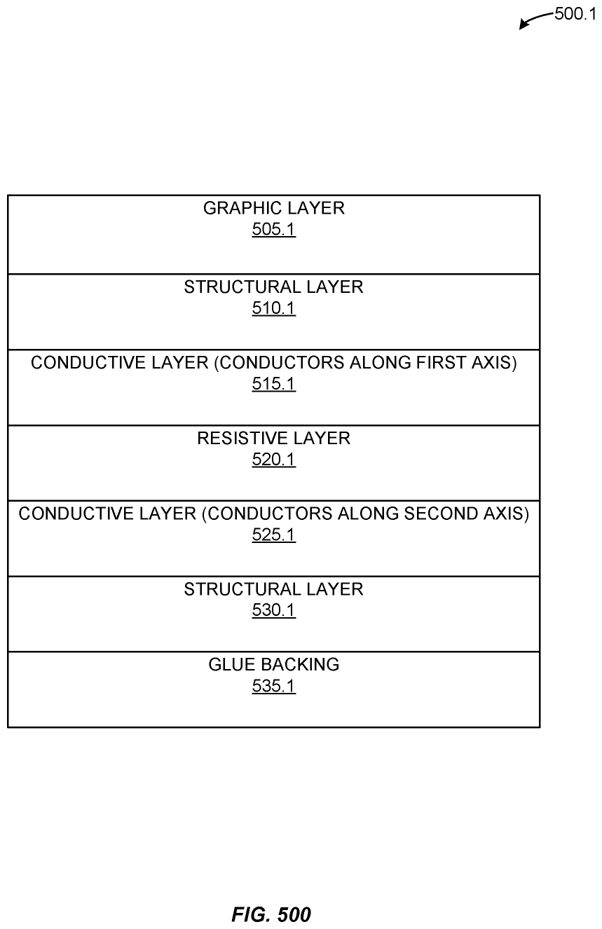

Smart floor tiles utilizing a variety of sensing technologies, such as membrane switches, pressure sensors and capacitive sensors, to identify instances of contact with a floor are within the contemplated scope of this disclosure. FIG. 500 illustrates aspects of a resistive smart floor tile 500.1 according to certain embodiments of the present disclosure. The embodiment of the resistive smart floor tile 500.1 shown in FIG. 500 is for illustration only and other embodiments could be used without departing from the scope of the present disclosure.

In the non-limiting example shown in FIG. 500, a cross section showing the layers of a resistive smart floor tile 500.1 is provided. According to some embodiments, the resistance to the passage of electrical current through the smart floor tile varies in response to contact pressure. From these changes in resistance, values corresponding to the pressure and location of the contact may be determined. In some embodiments, resistive smart floor tile 500.1 may comprise a modified carpet or vinyl floor tile, and have dimensions of approximately 2′×2′.

According to certain embodiments, resistive smart floor tile 500.1 is installed directly on a floor, with graphic layer 505.1 comprising the top-most layer relative to the floor. In some embodiments, graphic layer 505.1 comprises a layer of artwork applied to smart floor tile 500.1 prior to installation. Graphic layer 505.1 can variously be applied by screen printing or as a thermal film.

According to certain embodiments, a first structural layer 510.1 is disposed, or located, below graphic layer 505.1 and comprises one or more layers of durable material capable of flexing at least a few thousandths of an inch in response to footsteps or other sources of contact pressure. In some embodiments, first structural layer 510.1 may be made of carpet, vinyl or laminate material.

According to some embodiments, first conductive layer 515.1 is disposed, or located, below structural layer 510.1. According to some embodiments, first conductive layer 515.1 includes conductive traces or wires oriented along a first axis of a coordinate system. The conductive traces or wires of first conductive layer 515.1 are, in some embodiments, copper or silver conductive ink wires screen printed onto either first structural layer 510.1 or resistive layer 520.1. In other embodiments, the conductive traces or wires of first conductive layer 515.1 are metal foil tape or conductive thread embedded in structural layer 510.1. In the non-limiting example shown in FIG. 500, the wires or traces included in first conductive layer 515.1 are capable of being energized at low voltages on the order of 5 volts. In the non-limiting example shown in FIG. 500, connection points to a first sensor layer of another smart floor tile or to tile controller are provided at the edge of each smart floor tile 500.1.

In various embodiments, a resistive layer 520.1 is disposed, or located, below conductive layer 515.1. Resistive layer 520.1 comprises a thin layer of resistive material whose resistive properties change under pressure. For example, resistive layer 320.1 may be formed using a carbon-impregnated polyethylete film.

In the non-limiting example shown in FIG. 500, a second conductive layer 525.1 is disposed, or located, below resistive layer 520.1. According to certain embodiments, second conductive layer 525.1 is constructed similarly to first conductive layer 515.1, except that the wires or conductive traces of second conductive layer 525.1 are oriented along a second axis, such that when smart floor tile 500.1 is viewed from above, there are one or more points of intersection between the wires of first conductive layer 515.1 and second conductive layer 525.1. According to some embodiments, pressure applied to smart floor tile 500.1 completes an electrical circuit between a sensor box (for example, tile controller 425.1 as shown in FIG. 400) and smart floor tile, allowing a pressure-dependent current to flow through resistive layer 520.1 at a point of intersection between the wires of first conductive layer 515.1 and second conductive layer 525.1. The pressure-dependent current may represent a measurement of pressure and the measurement of pressure may be transmitted to the cloud-based computing system 116.1.

In some embodiments, a second structural layer 530.1 resides beneath second conductive layer 525.1. In the non-limiting example shown in FIG. 500, second structural layer 530.1 comprises a layer of rubber or a similar material to keep smart floor tile 500.1 from sliding during installation and to provide a stable substrate to which an adhesive, such as glue backing layer 535.1 can be applied without interference to the wires of second conductive layer 525.1.

The foregoing description is purely descriptive and variations thereon are contemplated as being within the intended scope of this disclosure. For example, in some embodiments, smart floor tiles according to this disclosure may omit certain layers, such as glue backing layer 535.1 and graphic layer 505.1 described in the non-limiting example shown in FIG. 500.

According to some embodiments, a glue backing layer 535.1 comprises the bottom-most layer of smart floor tile 500.1. In the non-limiting example shown in FIG. 500, glue backing layer 535.1 comprises a film of a floor tile glue.

FIG. 600 illustrates a master control device 600.1 according to certain embodiments of this disclosure. FIG. 600 illustrates a master control device 600.1 according to certain embodiments of this disclosure. The embodiment of the master control device 600.1 shown in FIG. 600 is for illustration only and other embodiments could be used without departing from the scope of the present disclosure.

In the non-limiting example shown in FIG. 600, master control device 600.1 is embodied on a standalone computing platform connected, via a network, to a series of end devices (e.g., tile controller 405A.1 in FIG. 400) in other embodiments, master control device 600.1 connects directly to, and receives raw signals from, one or more smart floor tiles (for example, smart floor tile 500.1 in FIG. 500). In some embodiments, the master control device 600.1 is implemented on a server 128.1 of the cloud-based computing system 116.1 in FIG. 100B and communicates with the smart floor tiles 112.1, the moulding sections 102.1, the camera 50.1, the computing device 12.1, the computing device 15.1, and/or the electronic device 13.1.

According to certain embodiments, master control device 600.1 includes one or more input/output interfaces (I/O) 605.1. In the non-limiting example shown in FIG. 600, I/O interface 605.1 provides terminals that connect to each of the various conductive traces of the smart floor tiles deployed in a physical space. Further, in systems where membrane switches or smart floor tiles are used as mat presence sensors, I/O interface 605 electrifies certain traces (for example, the traces contained in a first conductive layer, such as conductive layer 515.1 in FIG. 500) and provides a ground or reference value for certain other traces (for example, the traces contained in a second conductive layer, such as conductive layer 525.1 in FIG. 500). Additionally, I/O interface 605.1 also measures current flows or voltage drops associated with occupant presence events, such as a person's foot squashing a membrane switch to complete a circuit, or compressing a resistive smart floor tile, causing a change in a current flow across certain traces. In some embodiments, I/O interface 605.1 amplifies or performs an analog cleanup (such as high or low pass filtering) of the raw signals from the smart floor tiles in the physical space in preparation for further processing.

In some embodiments, master control device 600.1 includes an analog-to-digital converter (“ADC”) 610.1. In embodiments where the smart floor tiles in the physical space output an analog signal (such as in the case of resistive smart floor tile), ADC 610.1 digitizes the analog signals. Further, in some embodiments, ADC 610.1 augments the converted signal with metadata identifying, for example, the trace(s) from which the converted signal was received, and time data associated with the signal. In this way, the various signals from smart floor tiles can be associated with touch events occurring in a coordinate system for the physical space at defined times. While in the non-limiting example shown in FIG. 600, ADC 610.1 is shown as a separate component of master control device 600.1, the present disclosure is not so limiting, and embodiments wherein ADC 610.1 is part of, for example, I/O interface 605.1 or processor 615.1 are contemplated as being within the scope of this disclosure.

In various embodiments, master control device 600.1 further comprises a processor 615.1. In the non-limiting example shown in FIG. 6, processor 615 is a low-energy microcontroller, such as the ATMEGA328P by Atmel Corporation. According to other embodiments, processor 615.1 is the processor provided in other processing platforms, such as the processors provided by tablets, notebook or server computers.

In the non-limiting example shown in FIG. 600, master control device 600.1 includes a memory 620.1. According to certain embodiments, memory 620.1 is a non-transitory memory containing program code to implement, for example, APIs 625.1, networking functionality and the algorithms for generating and analyzing tracks and predicting/preventing fall events by performing interventions described herein.

Additionally, according to certain embodiments, master control device 600.1 includes one or more Application Programming Interfaces (APIs) 625.1. In the non-limiting example shown in FIG. 600, APIs 625.1 include APIs for determining and assigning break points in one or more streams of smart floor tile data and/or moulding section sensor data and defining data sets for further processing. Additionally, in the non-limiting example shown in FIG. 600, APIs 625.1 include APIs for interfacing with a job scheduler (for example, trigger controller 420.1 in FIG. 400) for assigning batches of data to processes for analysis and determination of tracks and predicting/preventing fall events using interventions. According to some embodiments, APIs 625.1 include APIs for interfacing with one or more reporting or control applications provided on a client device. Still further, in some embodiments, APIs 625.1 include APIs for storing and retrieving image data, smart floor tile data, and/or moulding section sensor data in one or more remote data stores (for example, database 430.1 in FIG. 400, database 129.1 in FIG. 100B, etc.).

According to some embodiments, master control device 600.1 includes send and receive circuitry 630.1, which supports communication between master control device 600.1 and other devices in a network context in which smart building control using directional occupancy sensing is being implemented according to embodiments of this disclosure. In the non-limiting example shown in FIG. 600, send and receive circuitry 630.1 includes circuitry 635.1 for sending and receiving data using Wi-Fi, including, without limitation at 900 MHz, 2.8 GHz and 5.0 GHz. Additionally, send and receive circuitry 630.1 includes circuitry, such as Ethernet circuitry 640.1 for sending and receiving data (for example, smart floor tile data) over a wired connection. In some embodiments, send and receive circuitry 630.1 further comprises circuitry for sending and receiving data using other wired or wireless communication protocols, such as Bluetooth Low Energy or Zigbee circuitry.

Additionally, according to certain embodiments, send and receive circuitry 630.1 includes a network interface 650.1, which operates to interconnect master control device 600.1 with one or more networks. Network interface 650.1 may, depending on embodiments, have a network address expressed as a node ID, a port number or an IP address. According to certain embodiments, network interface 650.1 is implemented as hardware, such as by a network interface card (NIC). Alternatively, network interface 650.1 may be implemented as software, such as by an instance of the java.net.NetworkInterface class. Additionally, according to some embodiments, network interface 650 supports communications over multiple protocols, such as TCP/IP as well as wireless protocols, such as 3G or Bluetooth.

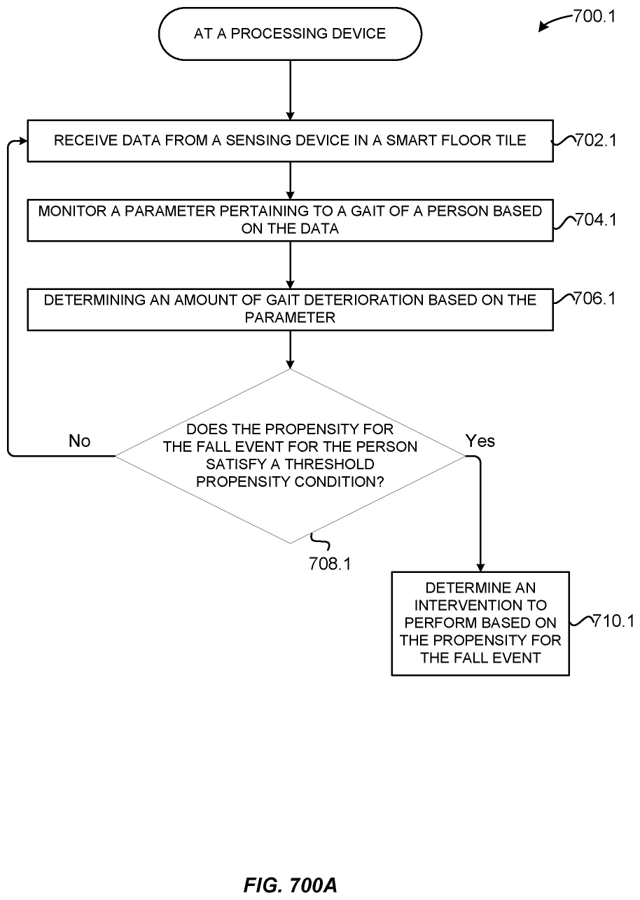

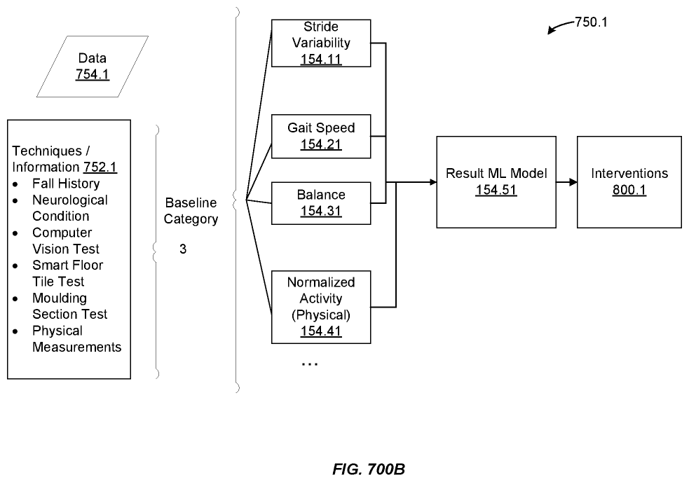

FIG. 700A illustrates an example of a method 700.1 for predicting a fall event according to certain embodiments of this disclosure. The method 700.1 may be performed by processing logic that may include hardware (circuitry, dedicated logic, etc.), software, or a combination of both. The method 700.1 and/or each of their individual functions, subroutines, or operations may be performed by one or more processors of a computing device (e.g., any component (server 128.1, training engine 152.1, machine learning models 154.1, etc.) of cloud-based computing system 116.1 of FIG. 100B) implementing the method 700.1. The method 700.1 may be implemented as computer instructions stored on a memory device and executable by the one or more processors. In certain implementations, the method 700.1 may be performed by a single processing thread. Alternatively, the method 700.1 may be performed by two or more processing threads, each thread implementing one or more individual functions, routines, subroutines, or operations of the methods.

At block 702.1, the processing device may receive data from a sensing device in a smart floor tile 112.1. The data may be pressure measured by a person stepping on the smart floor tile 112.1 with one or both of their feet. The data may include a specific coordinate where the pressure is measured (e.g., an identity of the sensing device that is pressed in the smart floor tile 112.1 may be included with the data and the location of that particular sensing device is stored in the database 129.1) by the sensing device, an amount of pressure applied to the sensing device, a time at which the pressure is applied to the sensing device, and so forth. In some embodiments, data may be received from the moulding section 102.1 and/or the camera 50.1. In embodiments where the parameter is monitored using the camera, the processing device may use computer vision, object recognition, measured pressure, location of feet of the person, or some combination thereof.

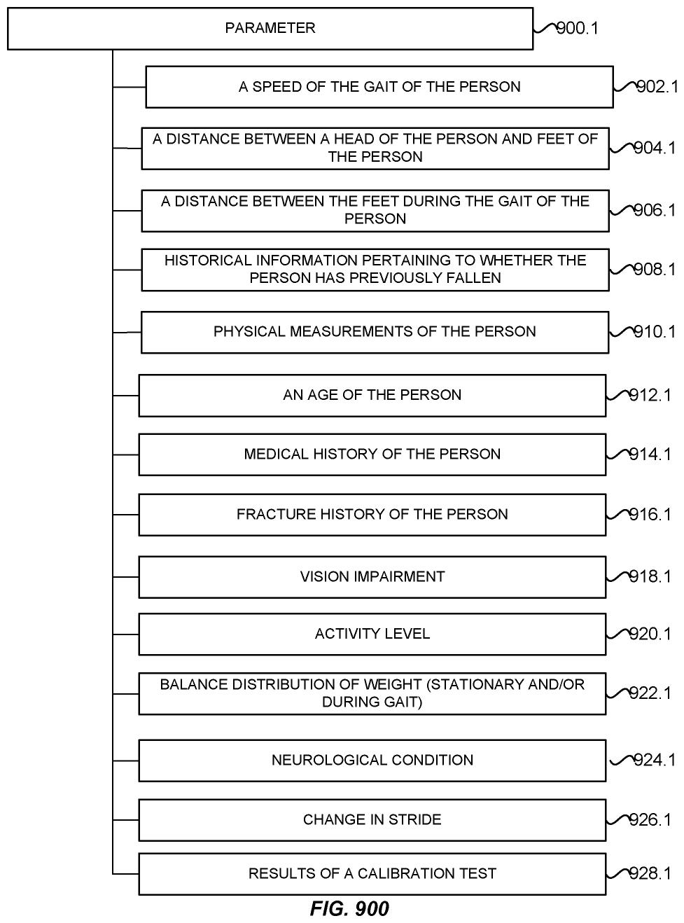

At block 704.1, the processing device may monitor a parameter pertaining to a gait of a person based on the data. The parameters are discussed in detail with regard to FIG. 900 below. Monitoring the parameter may include determining a category for the person based on the value of the parameter. The category may range from 1 to 5 where 1 is correlated with a least likely chance of the person falling and a 5 is correlated with a highest chance of the person falling. The person may be re-categorized while they are located in the physical space with the smart floor tiles 112.1, the moulding sections 102.1, and/or the camera 50.1. For example, the progression of the person from a category 1 to 5 for a propensity for a fall event to occur may be tracked and a time differential of how long it took for the person to move between categories may be determined and used to determine what intervention to perform. The categories for the propensity for the fall event may ebb and flow as the person improves and/or worsens a health condition and/or as their gait and/balance improve or worsen.

At an initial time, as described below, the person may be categorized for one or more parameters and the categories may serve as one or more gait baseline parameters to use to compare against categories that are assigned to the person for the one or more parameters at a later time. The one or more gait baseline parameters may be stored as part of a motion profile for the person in the database 129.1 of the cloud-based computing system 116.1. The motion profile may include an average gait speed of the person, paths the person takes during a day and the times at which the person takes those paths, average width of feet from each other during gait, length of stride, balance of the person based on distribution of weight between feet standing still and/or walking, and so forth.

However, in some instances, the person may not receive the one or more initial categories (gait baseline parameters). In such an embodiment, the processing device may use historical information pertaining to gait and/or balance that are characteristic of a propensity for a person to experience a fall event. The historical information may be obtained from a large group of people over a period of time and may be correlated with whether the people in the group experienced fall events. The historical information may be any combination of parameters including physical measurements (e.g., weight, height), personal statistics (e.g., age, gender, demographic information, etc.), medical history, neurological conditions, medications, fall history, gait characteristics (e.g., gait speed reduction within a certain time period, width of feet during gait, proximity of head to feet during gait, etc.), balance characteristics, and the like. For example, if the processing device determines the person has fallen in the past and the width of the person's feet are within a certain range, the processing device may determine the propensity for the person to experience a fall event warrants an intervention. Any suitable combination of historical information may be used to determine whether the person is likely to experience a fall event without using a gait baseline parameter.

At block 706.1, the processing device may determine an amount of gait deterioration based on the parameter. The amount of gait deterioration may be any suitable indication, such as a category (e.g., 1-5), a score (e.g., 1-5), a percentage (0-100%), and the like. In some embodiments, the amount of gait deterioration may be based on the category, score, or percentage for a particular parameter changing a certain amount within a certain time period. For example, the gait deterioration may be determined to be high if the category for a parameter changed from a 1 to a 5 within a short amount of time (e.g., minutes).

At block 708.1, the processing device may determine whether the propensity for the fall event for the person satisfies a threshold propensity condition based on (i) the amount of gait deterioration satisfying a threshold deterioration condition, or (ii) the amount of gait deterioration satisfying the threshold deterioration condition within a threshold time period. The propensity for a fall event may refer to a score (e.g., 1-5), a category (e.g., 1-5), percentage (e.g., 0-100%), or any suitable indication that is tied to how likely the person is to experiencing a fall event. The propensity for the fall event may be determined based on a category, score, or percentage for one parameter or any suitable combination of categories, scores, or percentages for parameters. For example, if the gait speed of the person deteriorated by 50% and the stride length of the person deteriorated by 50%, then the propensity for the fall event may be categorized at a high level (e.g., 4), and if the gait speed of the person deteriorated by 10% and the stride length of the person deteriorated by 5%, then the propensity for the fall event may be categorized a low level (e.g., 1).

In some embodiments, the threshold propensity condition may be satisfied when the amount of gait deterioration satisfies a threshold deterioration condition. For example, if the threshold deterioration condition specifies the amount of gait deterioration has to exceed a certain value (e.g., category of 3, score of 3, a percentage (50%), etc.) and the amount of gait deterioration exceeds the certain value, then the threshold propensity condition may be satisfied.

In some embodiments, the threshold propensity condition may be satisfied when the amount of gait deterioration satisfies a threshold deterioration condition within a threshold time period. For example, if the threshold deterioration condition specifies the amount of gait deterioration has to exceed a certain value (e.g., category of 3, score of 3, a percentage (50%), etc.) within the threshold time period (e.g., minutes, hours, days, etc.), and the amount of gait deterioration exceeds the certain value within that threshold time period (e.g., the amount of gait deterioration changed from 5% to 50% within an hour), then the threshold propensity condition may be satisfied.

If the propensity for the fall event for the person does not satisfy the threshold propensity condition, the processing device may return to block 702.1 to receive subsequent data from the sensing device in the smart floor tile 112.1 and continue to perform the other operations specified in the blocks 704.1, 706.1, and 708.1 until the propensity for the fall event for the person satisfies the threshold propensity condition.

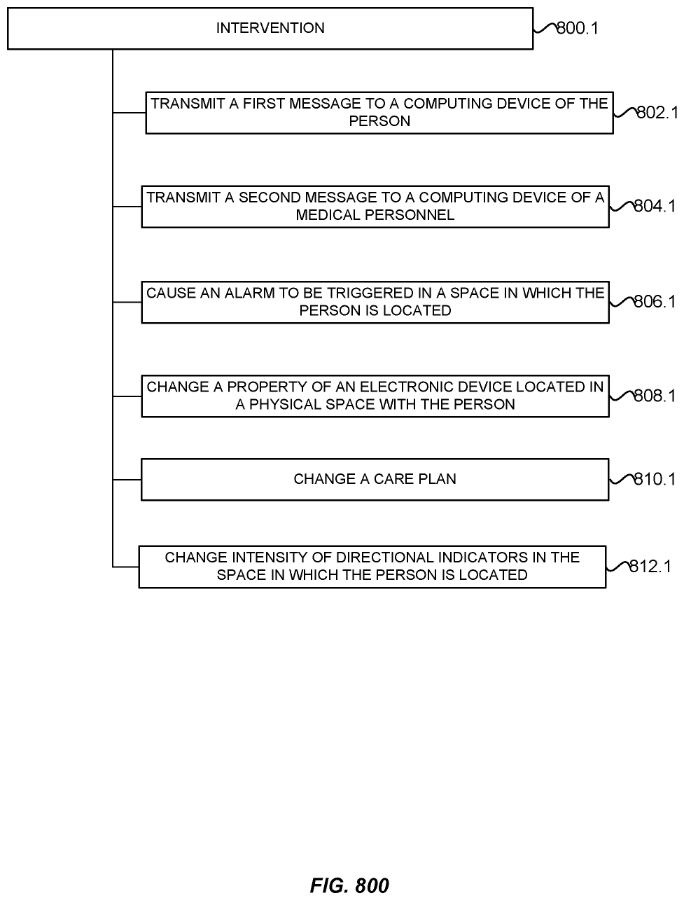

If the propensity for the fall event for the person satisfies the threshold propensity condition, then at block 710.1, the processing device determines an intervention to perform based on the propensity for the fall event. Various types of interventions are discussed in detail with regard to FIG. 800 below. There may be varying types of interventions with varying levels of severity that are associated with different levels of the propensity for the fall event. The interventions may escalate in severity based on how imminent the fall event is to occurring determined by the propensity for the fall event. Once one or more interventions are selected, the processing device may perform the one or more interventions.

In some embodiments, the monitoring the parameter pertaining to the gait of the person based on the data (block 704.1), the determining the amount of gait deterioration based on the parameter (block 706.1), and/or the determining whether the propensity for the fall event for the person satisfies the threshold propensity condition may include inputting the data into one or more machine learning models 154.1. The one or more machine learning models 154.1 may be trained to determine the amount of gait deterioration based on the parameter and to determine whether the propensity for the fall event for the person satisfies the threshold propensity condition.

In some embodiments, the effectiveness of the interventions that are performed may be tracked and a feedback loop may be used to update the one or more machine learning models 154.1. For example, the smart floor tiles 112.1, moulding sections 102.1, and/or camera 50.1 may obtain data that indicates whether the person fell or not after the intervention is performed. That data may be transmitted to the cloud-based computing system 116.1, which may update the machine learning models to either perform different interventions in the future if the intervention(s) performed did not work or continue to perform the same interventions if the interventions did work.

FIG. 700B illustrates an example architecture 750.1 including machine learning models 154.1 to perform the method of FIG. 700A according to certain embodiments of this disclosure. In some embodiments, each parameter that is monitored may be associated with a calibrated gait baseline parameter. The one or more gait baseline parameters may be combined using a function that weights the various gait baseline parameters to determine a baseline category, score, or percentage. Some embodiments may use certain information and/or techniques 752.1 when determining the one or more gait baseline parameters. Each of the gait baseline parameters may be stored in the database 129.1.

For example, the information and/or techniques 752.1 may include the fall history of the person. Research has shown that if a person has previously fallen, the person may be more likely to fall again in the future. The information and/or techniques 752.1 may include any neurological condition of the person. Certain neurological conditions may increase the likelihood that the person will fall. For example, if the person has epilepsy, the person may be prone to seizures that cause the person to fall while walking.

The information and/or techniques 752.1 may include a computer vision test. The camera 50.1 may stream video and/or images of the person during gait in a physical space (e.g., a care room). Using data received from the camera 50.1, the cloud-based computing system 116.1 may analyze the parameters of the person using computer vision to set the gait baseline parameters.

For example, computer vision may be used to determine an average gait stride length of the person, an average gait speed, an average width of feet from one another during gait, an average distance from a head of the person to the feet of the person, a balance of the person, whether the person gaits in a straight line, typical paths taken during gait, times at which the person gaits, average length of gait, and/or number of times the person gaits during a day, among others.

The information and/or techniques 752.1 may include a smart floor tile test. The smart floor tile test may involve receiving data from the smart floor tiles in the space in which the person is located while the person gaits. The data may include pressure measurements, location of pressure, time at which the pressure is measured, and so forth. The data may be used to determine an average gait stride length of the person, an average gait speed (e.g., differences in timestamps of detected footsteps from the smart floor tiles), an average width of feet from one another during gait, an average distance from a head of the person to the feet of the person, a balance of the person, whether the person gaits in a straight line, typical paths taken during gait, times at which the person gaits, average length of gait, and/or number of times the person gaits during a day, among others.

The information and/or techniques 752.1 may include moulding section testing. The moulding section test may involve receiving data from the moulding sections in the space in which the person is located while the person gaits. The data may include a silhouette of the person during the test as they gait in the space. The silhouette may be obtained using infrared imaging and/or proximity sensors that track the location of the person and the body parts of the person during the test as they gait. The data may be used to determine an average gait stride length of the person, an average gait speed (e.g., differences in timestamps of detected footsteps from the smart floor tiles), an average width of feet from one another during gait, an average distance from a head of the person to the feet of the person, a balance of the person, whether the person gaits in a straight line, typical paths taken during gait, times at which the person gaits, average length of gait, and/or number of times the person gaits during a day, among others.

In some embodiments, some combination of the computer vision test, the smart floor tile test, and/or the moulding section test may be used to calibrate the gait baseline parameters for the person.

The information and/or techniques 752.1 may include physical measurements of the person (e.g., height, weight, body weight distribution, body mass index, etc.) and other personal information about the person (e.g., age, medical history, gender, medications, and the like).

The one or more gait baseline parameters may be used in any combination to determine a baseline category for the propensity of the person to experience a fall event. In the depicted embodiment, the baseline category is determined to be a 3 in a range of 1-5 where 1 is the least likely to experience a fall event and a 5 is the most likely to experience a fall event. The one or more baseline parameters and/or the baseline category may be stored in the database 129.1.

The cloud-based computing system 116 may receive data 754.1 from the smart floor tiles 112.1, the moulding sections 102.1, and/or the camera 50.1. The data may be input into one or more machine learning models 154.1 that are each trained to monitor a particular parameter using the data and determine an amount of gait deterioration based on the monitored parameter. For example, the machine learning models 154 include a stride variability machine learning model 154.11, a walking speed machine learning model 154.21, a balance machine learning model 154.31, and a normalized activity (physical) machine learning mode 154.4.1. The machine learning models 154.11-154.41 may be trained to determine an amount of gait deterioration for a particular parameter. The amount of gait deterioration may include a category, a score, a rate, a percentage, or any suitable indicator the provides a measurement of the amount of gait deterioration.

The stride variability machine learning model 154.11 may be trained using training data that is labeled to indicate that stride variability, in terms of stride time (e.g., how long it takes a person to perform a stride during gait), stride length (e.g., a distance of a stride), or both, is correlated with a certain amount of gait deterioration. Further the stride variability machine learning model 154.11 may be trained to determine that the change in the characteristics of the stride occurring within certain periods of time is correlated with a certain amount of gait deterioration.

The gait speed machine learning model 154.21 may be trained using training data that is labeled to indicate that gait speed, in terms of how fast the person walks, is correlated with a certain amount of gait deterioration. Further the stride variability machine learning model 154.11 may be trained to determine that the change (e.g., reduction) in gait speed occurring within certain periods of time is correlated with a certain amount of gait deterioration.

The balance machine learning model 154.31 may be trained using training data that is labeled to indicate that the person is exhibiting a certain amount of balance is correlated with a certain amount of gait deterioration. The amount of balance may be measured in by body sway that may occur in any plane of motion. Sway may be determined based on analyzing the footsteps of the person and/or distribution of weight of the person as detected by the smart floor tiles 112.1, by analyzing body motion using video data from the camera 50.1 and/or data obtained from the moulding sections 102.1. Impaired balance may be used to predict the propensity for the fall event to occur. Further the stride variability machine learning model 154.11 may be trained to determine that the change in the balance of the person occurring within certain periods of time is correlated with a certain amount of gait deterioration.

The normalized activity machine learning model 154.21 may be trained using training data that is labeled to indicate that certain physical traits of a person are correlated with a certain amount of gait deterioration. For example, changes in the height, weight, age, weight distribution, body mass index, medical conditions, fall history, activity levels, and the like, may contribute to gait deterioration. Further the normalized activity machine learning model 154.11 may be trained to determine that the change in the physical traits occurring within certain periods of time is correlated with a certain amount of gait deterioration.

As depicted, any suitable number of machine learning models 154 (up to parameter machine learning model N) may be trained and used to determine the amount of gait deterioration as it pertains to a particular parameter. The output of the machine learning models 154.11 through 154.41 associated with the respective parameters may be input to a result machine learning model 154.51.

The result machine learning model 154.51 may be trained to analyze the various amounts of gait deterioration for the respective parameters represented by the respective machine learning models 154.11-154.41 and determine a propensity for the fall event. In some embodiments, the amount of gait deterioration for each parameter that is output by the machine learning models 154.11-154.41 may be compared with a respective corresponding gait baseline parameter when determining the propensity for the fall event. Each amount of gait deterioration may be considered a flag if the amount of gait deterioration satisfies a threshold deterioration condition. In some embodiments, the larger the number of flags that are present for the person, the higher the propensity for the fall event to occur for the person. That is, if there are flags present for the amount of gait deterioration determined by the stride variability machine learning model 154.11, the gait speed machine learning model 154.21, the balance machine learning model 154.31, and the normalized activity machine learning model 154.41, then the propensity for the fall event for the person may be high. In contrast, if there is just one flag present for the stride variability machine learning model 154.11, then the propensity for the fall event may be low.

In some embodiments, the propensity for the fall event may be compared with the baseline category to determine whether the propensity for the fall event satisfies the threshold propensity condition. For example, if the propensity for the fall event varies from the baseline category by a threshold amount (e.g., 1, 2, 3, etc.), then the propensity for the fall event may satisfy the threshold propensity condition.

Further, some machine learning models 154.11-154.41 may be associated with higher priority parameters and their output may be weighted differently when compared with the output of the other machine learning models corresponding to lesser priority parameters. For example, balance may be considered a high priority flag in indicating a fall event, and thus, the amount of gait deterioration determined for balance by the balance machine learning model 154.31 may be weighted more heavily that outputs of the other machine learning models 154.11, 154.21, and/or 154.41.

The result machine learning model 154.51 may also determine one or more interventions to perform based on the propensity for the fall event for the person. More severe interventions may be selected if the propensity for the fall event is high, and less severe interventions may be selected if the propensity for the fall event is low.

FIG. 800 illustrates example interventions 800.1 according to certain embodiments of this disclosure. The interventions 800.1 may each be associated with a level of severity. Less severe interventions 800.1 may be selected and performed for people having lower propensity for a fall event to occur, and more severe interventions 800.1 may be selected and performed for people having higher propensity for the fall event to occur. The interventions 800.1 are provided as examples and are not intended to limit the scope of the disclosure. Additional interventions 800.1 or fewer interventions 800.1 may be used in some embodiments.

A first intervention 802.1 may include transmitting a message to a computing device 12.1 of the person (e.g., elderly patient) for which the propensity of the fall event satisfies the threshold propensity condition. The message may include a notification that the fall event is likely to occur and/or instructs the user to stop walking, grab onto a supporting structure, change a gait speed, change the width of their feet, change their distribution of weight, and the like.

A second intervention 804.1 may include transmitting a message to a computing device of the medical personnel (e.g., nurse) that is on duty and/or assigned to care for the person. For example, the message may include a notification to the medical personnel that indicates the person is about to experience a fall event. The message may include a name of the person, which room the person is located, and/or a likelihood that the person is going to fall, among other things. For example, the message may include information about previous fall history for the person, known medical conditions of the person, fracture history of the person, age, medications taken by the person, and/or any suitable information that may aid the medical personnel in treating the person if the fall event occurs before the medical personnel arrives and/or if the medical personnel is able to prevent the fall. In some embodiments, the message may include a notification that reassigns the medical personnel to a station in closer proximity to or in farther proximity from the room where the person is located.

A third intervention 806.1 may causing an alarm to be triggered in a space in which the person is located. The alarm may be disposed at a nursing station that emits a certain audible, visual, and/or haptic indication that is represents the fall event may occur. The alarm may be disposed in the room in which the person is located and may emit a certain audible, visual, and/or haptic indication that is represents the fall event may occur.

A fourth intervention 808.1 may include changing a property of an electronic device located in a physical space with the person. For example, a smart light installed in the room in which the person is located may be controlled to emit a certain color of light and/or pattern of light, a smart thermostat may be controlled to change a temperature, a smart device located on the floor (e.g., smart vacuum) may be controlled to return to its home base to clear the way for the person to gait, a smart speaker may be controlled to play music and/or emit a warning about the fall event, and the like.

A fifth intervention 810.1 may include changing a care plan for the person. The care plan may be changed to instruct the person to complete a puzzle within a certain time period and/or perform any mentally stimulating activity that is correlated with improved mental capabilities. Improving mental capabilities may aid in reducing the likelihood of the person experiencing a fall event. The change in the care plan may relate to a diet of the person, different medication to prescribe to the person, an activity plan for the person, laboratory tests to perform for the person, medical examinations to perform for the person, and so forth.

A sixth intervention 812.1 may include changing an intensity of one or more directional indicators in the space in which the person is located. In some embodiments, the directional indicators may be lights, a display, audio speakers, and the like that are included in the moulding sections 102.1. In some embodiments, the directional indicators may be any suitable electronic device in the space in which the person is located that is capable of providing an indication of a direction for the person to move.

FIG. 900 illustrates example parameters 900.1 that may be monitored according to certain embodiments of this disclosure. Some of the parameters may have higher priority in terms of indicating whether a fall event may occur and those parameters may receive a higher weight when determining the propensity for the fall event. The parameters 900.1 are provided as examples and are not intended to limit the scope of the disclosure. Additional parameters 900.1 or fewer parameters 900.1 may be used in some embodiments.

A first parameter 902.1 may include a speed of the gait of the person. Gait speed may be determined based on the footsteps and how quickly the footsteps are made using the data from the smart floor tile 112.1, the moulding sections 102.1, and/or the camera 50.1. For example, the impression tile data received from the smart floor tile 112.1 may include the measured pressure associated with the footsteps and timestamps at which the pressure is measured. Such timestamps may be used to determine the speed at which the person is walking. Research has shown that reduced gait speed is an indicator of a propensity for a fall event.

A second parameter 904.1 may include a distance between a head of the person and feet of the person. Data received from the camera 50.1 and/or the moulding sections 102.1 may be used to determine the distance between the head of the person and feet of the person. Research has shown that the closer a person's head is to their feet, the more likely they are to fall because their center of gravity is off balance. As people age, their posture tends to decline and their heads often get closer to their feet as they hunch over. A reduction in distance between the head and feet of a person is an indicator of a propensity for a fall event.

A third parameter 906.1 may include a distance between the feet of the person during the gait of the person. The distance may be a width between the left and right foot. The distance may be a length of the stride between the left and right foot. If the width of the feet reduces, research has shown that is an indicator for a propensity for a fall event.

A fourth parameter 908.1 may include historical information pertaining to whether the person has previously fallen. Research shows that a person is more likely to fall again if that person has already experienced a fall event in the past.

A fifth parameter 910.1 may include physical measurements of the person. For example, the physical measurements may include height, weight, body mass index, weight distribution, and so forth. Certain physical measurements may be indicative of a propensity for a fall event to occur.

A sixth parameter 912.1 may include an age of the person. Research shows people over a certain age (e.g., 60) are more likely to experience a fall event because their muscles and skeletal strength weakens.

A seventh parameter 914.1 may include a medical history of the person. For example, if the person has a disease or medical condition, then that may indicate a propensity for a fall event.

An either parameter 916.1 may include a fracture history of the person. For example, if the person has previously fractured their hip, then that may indicate a propensity for a fall event.

A ninth parameter 918.1 may include vision impairment of the person. For example, if the person has poor eyesight, then that may indicate a propensity for a fall event (e.g., the person may not be able to see the floor is wet).

A tenth parameter 920.1 may include an activity level of the person. For example, if the person is rarely active, then their muscles may be atrophied. As a result, the person may be more likely to experience a fall event if they are not active.

An eleventh parameter 922.1 may include a balance distribution of weight for the person when the person is stationary and/or during gait. The balance distribution of weight for the person may be measured when they are stationary using the smart floor tiles 112.1 by measuring the pressure applied to the smart floor tiles 112.1 by the left foot and right foot. If the balance distribution of weight changes by a threshold amount while stationary, it may indicate that the person is going to experience a fall event. Further, the balance distribution of weight for the person may be measure as the person gaits by measuring the pressure applied by the left foot and the right foot to the smart floor tiles 112.1. If the balance distribution of weight changes for the left foot or the right foot, that may indicate the person is swaying and is losing their balance and is likely to experience a fall event.

In some embodiments, historical information may be referenced that indicates people having certain physical measurements (e.g., height, weight, etc.) at certain ages typically have certain balance distribution of weight while stationary and during gait. In such an embodiment, gait baseline parameters may not be used and the historical information may be used to determine whether balance distribution of weights for people with similar physical measurements and age match are different by a threshold amount. If the balance distribution of weights differ by the threshold amount, then the person is likely to experience a fall event.

A twelfth parameter 924.1 may include a neurological condition of the person. Certain neurological conditions indicate a propensity for a fall event. For example, epilepsy, alzheimers, etc. may increase the chances of a person experiencing a fall event.

A thirteenth parameter 926.1 may include a change in stride of the person. Reduction in the length of stride of the person may indicate a propensity for a fall event. Also, reduction in stride time may indicate a propensity for the fall event.

A fourteenth parameter 928.1 may include a results of a calibration test. The calibration test may include the computer vision test, the smart floor tile test, and/or the moulding section test.

FIG. 1000 illustrates an example of a method 1000.1 for using gait baseline parameters to determine an amount of gait deterioration according to certain embodiments of this disclosure. The method 1000.1 may be performed by processing logic that may include hardware (circuitry, dedicated logic, etc.), software, or a combination of both. The method 1000.1 and/or each of their individual functions, subroutines, or operations may be performed by one or more processors of a computing device (e.g., any component (server 128.1, training engine 152.1, machine learning models 154.1, etc.) of cloud-based computing system 116.1 of FIG. 100B) implementing the method 1000.1. The method 1000.1 may be implemented as computer instructions stored on a memory device and executable by the one or more processors. In certain implementations, the method 1000.1 may be performed by a single processing thread. Alternatively, the method 1000.1 may be performed by two or more processing threads, each thread implementing one or more individual functions, routines, subroutines, or operations of the methods.

At block 1002.1, the processing device may calibrate one or more gait baseline parameters for the person. Each gait baseline parameter may correspond with a separate respective parameter 900.1 that is monitored by the cloud-based computing system 116.1. The one or more gait baseline parameters may be stored in the database 129.1.

At block 1004.1, the processing device may determine the amount of gait deterioration based on comparing the parameter to at least one of the one or more gait baseline parameters. If the parameter varies by a certain amount or by the certain amount with a threshold period of time, then a certain amount of gait deterioration may be determined.

FIG. 1100 illustrates an example of a method for subtracting data associated with certain people from gait analysis according to certain embodiments of this disclosure. The method 1100.1 may be performed by processing logic that may include hardware (circuitry, dedicated logic, etc.), software, or a combination of both. The method 1100.1 and/or each of their individual functions, subroutines, or operations may be performed by one or more processors of a computing device (e.g., any component (server 128.1, training engine 152.1, machine learning models 154.1, etc.) of cloud-based computing system 116.1 of FIG. 100B) implementing the method 1100.1. The method 1100.1 may be implemented as computer instructions stored on a memory device and executable by the one or more processors. In certain implementations, the method 1100.1 may be performed by a single processing thread. Alternatively, the method 1100.1 may be performed by two or more processing threads, each thread implementing one or more individual functions, routines, subroutines, or operations of the methods.

For purposes of clarity, FIGS. 1100 and 1200A-B are disclosed together below. FIGS. 1200A-B illustrate an overhead view of an example for subtracting data associated with certain people from gait analysis according to certain embodiments of this disclosure. Each square 1200.1 in FIGS. 1200A-B represent a smart floor tile 112.1.

At block 1102.1, the processing device may determine an identity of a person (e.g., a medical personnel) in a physical space (e.g., a care room in a care facility where an elderly person is located). For example, the person may scan and/or swipe an identity badge at a reader 1206.1 disposed at an entry way (e.g., door) of the physical space in FIG. 1200A. The data read by the reader 1206.1 may include the identity of the person, a user identification number, a job title, and the like. The data read may be transmitted by the reader 1206.1 to the cloud-based computing system 116.1. In some embodiments, the reader 1206.1 may be a camera and may be capable of performing facial recognition techniques on an image of the person to determine the identity of the person and/or transmit an image of the person to the cloud-based computing system 116.1 that is capable of performing facial recognition techniques on the image to determine the identity of the person.

At block 1104.1, the processing device may receive data pertaining to a gait of the person. The person may walk from a first position 1204.11 to a second position 1204.21 as depicted in FIG. 1200A. The path of the person may be tracked based on data received via the smart floor tiles 112.1, the camera 50.1, and/or the moulding sections 102.1.

At block 1106.1, the processing device may correlate the data with the identity of the person. The correlated data with the identity of the person may be stored in the database 129.1.

At block 1108.1, the processing device may subtract the data during gait analysis of second data correlated with a second identity of a second person (e.g., an elderly person) in the physical space. For example, the person may walk from a first position 1202.11 to a second position 1202.21 in FIG. 1200A. It may be desirable to just analyze the path of the person who may be a target person (e.g., elderly person in a care facility) and not the path of the medical personnel (e.g., nurse) entering the room. Subtracting the data correlated with the identity of the first person removes that data from the gait analysis of the second data correlated with the second identity of the second person, as depicted in FIG. 1200B.

FIG. 1300 illustrates an example of a method 1300.1 for controlling an environment using a moulding section based on data received from a sensor of the moulding section according to certain embodiments of this disclosure. The method 1300.1 may be performed by processing logic that may include hardware (circuitry, dedicated logic, etc.), software, or a combination of both. The method 1300.1 and/or each of their individual functions, subroutines, or operations may be performed by one or more processors of a computing device (e.g., any component (server 128.1, training engine 152.1, machine learning models 154.1, etc.) of cloud-based computing system 116.1, the smart floor tile 112.1, and/or the moulding section 102.1 of FIG. 100B) implementing the method 1300.1. The method 1300.1 may be implemented as computer instructions stored on a memory device and executable by the one or more processors. In certain implementations, the method 1300.1 may be performed by a single processing thread. Alternatively, the method 1300.1 may be performed by two or more processing threads, each thread implementing one or more individual functions, routines, subroutines, or operations of the methods.

At block 1302.1, the processing device may receive data from a sensor in the moulding section 102.1. In some embodiments, the sensor may be any suitable proximity (e.g., optical, laser, haptic, etc.) sensor.

At block 1304.1, the processing device may determine, based on the data, whether a person is near the sensor.

At block 1306.1, the processing device may determine an operating state of a device 201.1 included in the moulding section 102.1. The device 201.1 may perform environment control of a physical space in which the moulding section is located. The device may be any suitable fan (e.g., electric fan) configurable to be included at least partially in the section moulding. For example, the device may be any suitable axial fan, centrifugal fan, mixed flow fan, and/or cross-flow fan. The device 201.1 may be communicatively coupled to a processing device of the moulding section 102.1, which may be further communicatively coupled to the cloud-based computing system 116.1.

The operating state may include active or inactive. Further, in some embodiments, the operating state may further include a mode such as heating, cooling, or venting. The operating state may include additional information such as a hold temperature, a home status, an away status, a person present status, an occupied status, or the like. The operating state may also include other information such as a user profile of the person detected to be in the physical space where the moulding section 102 is located. In some embodiments, the user profile may track the occupancy behavior of the user in the physical space and may further include temperature preferences of the user in a schedule used to control the device.

At block 1308.1, responsive to determining that the person is near the sensor and the operating state (e.g., inactive, set at a certain temperature) of the device, the processing device may change the device to operate in a second operating state (e.g., active, change temperature setting) to change a temperature of the physical space in which the moulding section is located.

In some embodiments, the processing device may receive second data from a second sensor (e.g, thermometer) in the moulding section. The processing device may determine, based on the second data, the temperature of the environment in which the moulding section is located. The processing device may determine whether the temperature satisfies a threshold temperature condition. Responsive to determining the temperature satisfies the threshold temperature condition, the processing device may change the operating state of the device to change the temperature of the physical space in which the moulding section is located.

In some embodiments, the processing device may receive second data from the proximity sensor in the moulding section. The processing device may determine, based on the second data, that the person is not near the sensor. The processing device may determine the second operating state (e.g., active, a particular mode (cool, heat, vent, etc.)) of the device included in the moulding section. Responsive to determining that the person is not near the sensor and the second operating state of the device, the processing device may change the device to operate in the operating state (e.g., inactive) to change a temperature of the physical space in which the moulding section is located.

In some embodiments, the processing device may receive an instruction sent from a computing device 12.1 external to the moulding section 102.1. The computing device 12.1 may be the user that occupies the physical space in which the moulding section 102.1 is located. For example, the user may use an application executing on the computing device 12.1 to cause the computing device 12.1 to transmit the instruction (e.g., activate, deactivate, set a certain temperature, etc.) to the cloud-based computing system 116.1 (which communicates the instruction to the moulding section 102.1) and/or directly to the moulding section 102.1.

In some embodiments, the processing device may determine whether the device 201.1 is operating in a certain operating state (e.g., active, inactive, heating, cooling, venting, etc.) for a threshold period of time. Responsive to determining the device is operating in the second operating state for the threshold period of time, the processing device may change the device 201.1 to operate in a different operating state (e.g., active, inactive, heating, cooling, venting, etc.).

FIG. 1400 illustrates an example of a method for controlling an environment using a moulding section based on data received from a smart floor tile according to certain embodiments of this disclosure. The method 1400.1 may be performed by processing logic that may include hardware (circuitry, dedicated logic, etc.), software, or a combination of both. The method 1400.1 and/or each of their individual functions, subroutines, or operations may be performed by one or more processors of a computing device (e.g., any component (server 128.1, training engine 152.1, machine learning models 154.1, etc.) of cloud-based computing system 116.1, the smart floor tile 112.1, and/or the moulding section 102.1 of FIG. 100B) implementing the method 1400.1. The method 1400.1 may be implemented as computer instructions stored on a memory device and executable by the one or more processors. In certain implementations, the method 1400.1 may be performed by a single processing thread. Alternatively, the method 1400.1 may be performed by two or more processing threads, each thread implementing one or more individual functions, routines, subroutines, or operations of the methods.

The operations of method 1400.1 may be performed in any suitable combination with the operations of method 1300.1 discussed above.

At block 1402.1, the processing device may receive data from a sensor in a smart floor tile 112.1. The sensor may be a pressure sensor capable of measuring an amount of pressure exerted on the smart floor tile 112.1. The measured pressure may be transmitted to the cloud-based computing system 116.1 and/or the moulding section 102.1.

At block 1404.1, the processing device may determine, based on the data, whether a person is present in a physical space including the smart floor tile 112.1. For example, the processing device may determine the person is present based on a certain amount of measured pressure. In some embodiments, the cloud-based computing system 116.1 may store weights associated with people that access the physical space. The measured pressure may be translated into an amount of weight that can be correlated with the stored weights for the people. In such a way, the processing device may determine an identity of which person of a set of people is in the room. In other instances, facial recognition may be performed on video data captured from camera 50.1 to determine an identity of a person in the physical space. Using the identity of the person, a user profile for temperature preferences at certain times of day may be accessed and used to control the device 201.1 in the moulding section 102.1. In some embodiments, the processing device may determine that there is a person present in the physical space and change the operating state of the device 201.1 without determining the identity of the person.

At block 1406.1, the processing device may determine an operating state of the device 201.1 including in a moulding section 102.1. The device 201.1 may perform environment control of the physical space in which the moulding section 102.1 is located. The operating state may include active or inactive. Further, in some embodiments, the operating state may further include a mode such as heating, cooling, or venting. The operating state may include additional information such as a hold temperature, a home status, an away status, a person present status, an occupied status, or the like. The operating state may also include other information such as a user profile of the person detected to be in the physical space where the moulding section 102.1 is located. In some embodiments, the user profile may track the occupancy behavior of the user in the physical space and may further include temperature preferences of the user in a schedule used to control the device. The operating state may be stored in the database 129.1 of the cloud-based computing system 116.1. In some instances, the cloud-based computing system 116.1 may query the moulding section 102.1 to provide the operating state of the device 201.1. Further, the moulding section 102.1 may push the operating state of the device 201.1 to the cloud-based computing system 116.1 periodically, continuously, on-demand, or when the operating state changes.

At block 1408.1, responsive to determining that the person is present in the physical space and the operating state of the device, the processing device may change the device 201.1 to operate in a different operating state to change a temperature of the physical space. For example, the processing device may determine the person is present and the operating state of the device 201.1 is inactive. In such a scenario, the processing device may cause the operating state of the device 201.1 to change to active, to cool the temperature of the physical space, for example.

In some embodiments, the processing device may receive second data from a second sensor (e.g., thermometer) in the moulding section 102.1. The processing device may determine, based on the second data, the temperature of the environment in which the moulding section 102.1 is located. The processing device may determine whether the temperature satisfies a threshold temperature condition. The temperature may satisfy the threshold temperature condition when the temperature is less than or equal to a certain temperature, greater than or equal to a certain temperature, or the like. The threshold temperature condition may be configured by a user using an application executing on the computing device 12.1. Responsive to determining the temperature satisfies the threshold temperature condition, the processing device may change the operating state of the device 201.1 to change the temperature of the physical space in which the moulding section 102.1 is located.

In some embodiments, the processing device may receive second data (e.g., pressure measurements) from the pressure sensor in the smart floor tile 112.1. The processing device may determine, based on the second data, that the person is not present in the physical space. The processing device may determine the second operating state of the device 201.1 included in the moulding section 102.1. Responsive to determining that the person in the physical space and the second operating state of the device, the processing device may change the device 201.1 to operating in a different operating state to change a temperature of the physical space in which the moulding section 102.1. For example, when the person leaves the physical space, based on the second data, the processing device may change the operating state to inactive.

In some embodiments, the processing device may operate a subset of devices 201.1 in a subset of moulding sections 102.1 of a superset of moulding sections 102.1 in a physical space based on tracking the location of the user in the physical space. For example, pressure measurements obtained from the smart floor tiles 112.1 and/or proximity measurements from the moulding sections 102.1 may enable tracking the presence of the user throughout a physical space. Just the devices 201.1 in the moulding sections 102.1 within a threshold distance (e.g., 1 foot, 2 feet, 3 feet, etc.) from the presence of the user may be activated or deactivated to provide a desired temperature to the environment of the physical space. In such an embodiment, the temperature of the environment may be more granularly and accurately controlled to provide an enhanced level of comfort to the user. This technique may enable efficiently controlling the use of the devices 201.1 to manage power consumption, as well. Selectively operating the devices 201.1 based on proximity of the user to the moulding sections 102.1 may extend the life of the devices 201.1 by reducing wear and tear.

FIG. 1500 illustrates an example physical space (e.g., first room 21.1) having an environment controlled by a set of moulding sections 102 (102.11-102.41) according to certain embodiments of this disclosure. Each of the moulding sections 102.1 may include one or more respective devices 201.1 (e.g., fans) (201.11-201.21) that may be individually controlled by the cloud-based computing system 116.1. The location of the user 25.1 may be tracked by the cloud-based computing system 116.1 in the first room 21.1 using the smart floor tiles 112.1, the moulding sections 102.1, and/or the camera 50.1.

In some embodiments, the cloud-based computing system 116.1 may cause the operating states of the devices 201.1 to change. For example, when the user 25.1 enters the first room 21.1, the operating states of one or more of the devices 201.1 may be changed from inactive operating state to active operating state to change the temperature of the environment in the first room 21.1. The identity of the user may be determined and a user profile may be reference to determine what temperature to set for the device 201.1 to produce and/or what operating state to instruct the devices to operate in.

Using the location of the user 25.1, the cloud-based computing system 116.1 may control a subset of the moulding sections 102.1. For example, because the user 25.1 is near the moulding sections 102.11 and 102.21, the cloud-based computing system 116.1 may cause the devices 201.11 and 201.21 to operate in an active operating state. The active operating state may cause the devices 201.11 and 201.21 to produce air or wind, as depicted by the dotted triangle 1500.1. However, because the user is not located near the moulding sections 102.31 or 102.41, the cloud-based computing system 116.1 may not change the operating state of the devices 201.1 included in those moulding sections 102.31 or 102.41.

FIG. 1600 illustrates an example computer system 1600.1, which can perform any one or more of the methods described herein. In one example, computer system 1600.1 may include one or more components that correspond to the computing device 12.1, the computing device 15.1, one or more servers 128.1 of the cloud-based computing system 116.1, the electronic device 13.1, the camera 50.1, the moulding section 102.1, the smart floor tile 112.1, or one or more training engines 152.1 of the cloud-based computing system 116.1 of FIG. 100A. The computer system 1600.1 may be connected (e.g., networked) to other computer systems in a LAN, an intranet, an extranet, or the Internet. The computer system 1600.1 may operate in the capacity of a server in a client-server network environment. The computer system 1600.1 may be a personal computer (PC), a tablet computer, a laptop, a wearable (e.g., wristband), a set-top box (STB), a personal Digital Assistant (PDA), a smartphone, a camera, a video camera, or any device capable of executing a set of instructions (sequential or otherwise) that specify actions to be taken by that device. Some or all of the components computer system 1600.1 may be included in the camera 50.1, the moulding section 102.1, and/or the smart floor tile 112.1. Further, while only a single computer system is illustrated, the term “computer” shall also be taken to include any collection of computers that individually or jointly execute a set (or multiple sets) of instructions to perform any one or more of the methods discussed herein.

The computer system 1600.1 includes a processing device 1602.1, a main memory 1604.1 (e.g., read-only memory (ROM), solid state drive (SSD), flash memory, dynamic random access memory (DRAM) such as synchronous DRAM (SDRAM)), a static memory 1606.1 (e.g., solid state drive (SSD), flash memory, static random access memory (SRAM)), and a data storage device 1608.1, which communicate with each other via a bus 1610.1.

Processing device 1602.1 represents one or more general-purpose processing devices such as a microprocessor, central processing unit, or the like. More particularly, the processing device 1602.1 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1602 may also be one or more special-purpose processing devices such as an application specific integrated circuit (ASIC), a field programmable gate array (FPGA), a digital signal processor (DSP), network processor, or the like. The processing device 1602.1 is configured to execute instructions for performing any of the operations and steps discussed herein.

The computer system 1600.1 may further include a network interface device 1612.1. The computer system 1600.1 also may include a video display 1614.1 (e.g., a liquid crystal display (LCD) or a cathode ray tube (CRT)), one or more input devices 1616.1 (e.g., a keyboard and/or a mouse), and one or more speakers 1618.1 (e.g., a speaker). In one illustrative example, the video display 1614.1 and the input device(s) 1616.1 may be combined into a single component or device (e.g., an LCD touch screen).

The data storage device 1616.1 may include a computer-readable medium 1620.1 on which the instructions 1622.1 embodying any one or more of the methodologies or functions described herein are stored. The instructions 1622.1 may also reside, completely or at least partially, within the main memory 1604.1 and/or within the processing device 1602.1 during execution thereof by the computer system 1600.1. As such, the main memory 1604.1 and the processing device 1602.1 also constitute computer-readable media. The instructions 1622.1 may further be transmitted or received over a network via the network interface device 1612.1.

While the computer-readable storage medium 1620.1 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

### Security System Implemented in a Physical Space Using Smart Floor Tiles

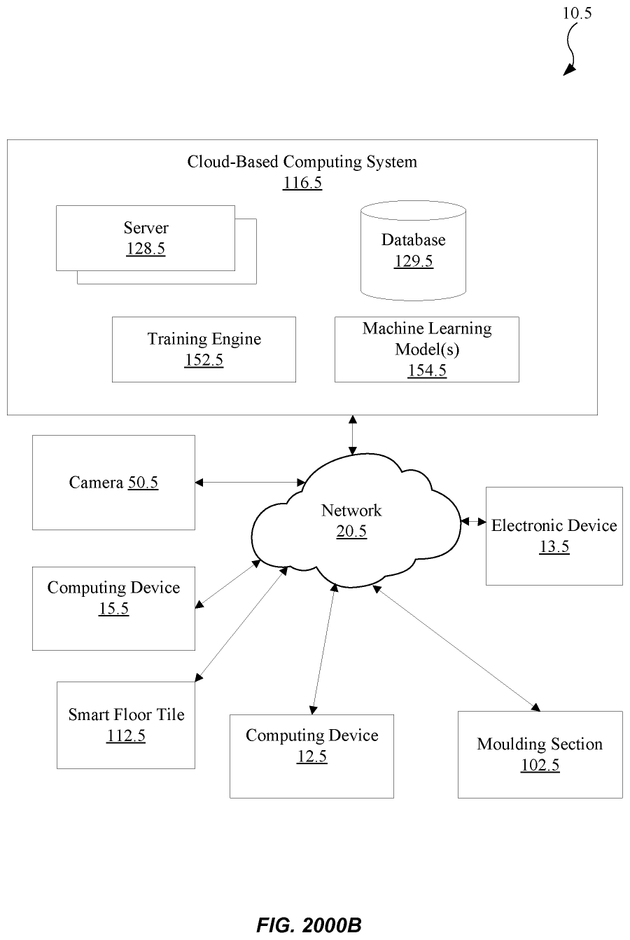

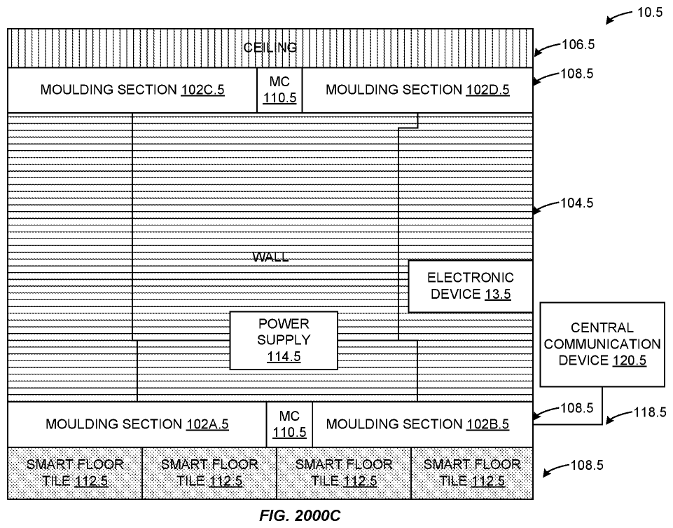

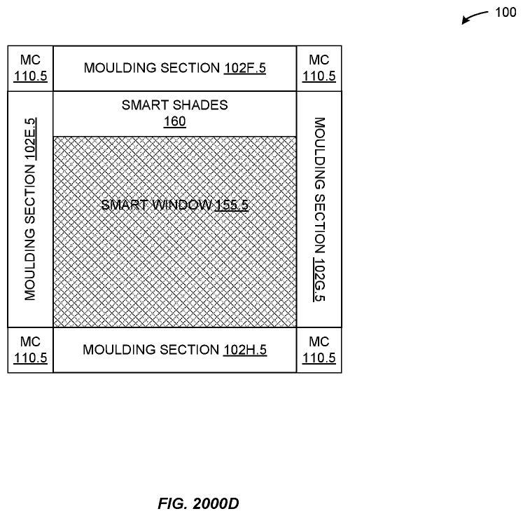

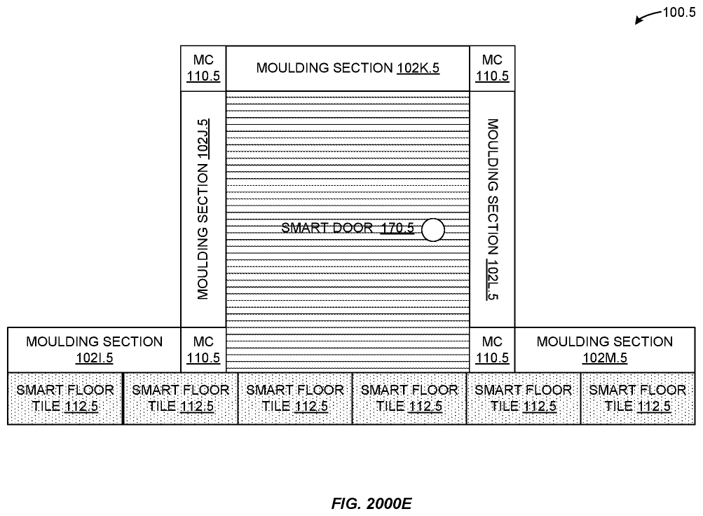

FIGS. 2000A through 19000, discussed below, and the various embodiments used to describe the principles of this disclosure in this patent document are by way of illustration only and should not be construed in any way to limit the scope of the disclosure.

Embodiments as disclosed herein relate to path analytics for objects in a physical space. For example, the physical space may be a convention center, or any suitable physical space where people move (e.g., walk, use a wheel chair or motorized cart, etc.) around in a path. At conventions, certain booths may be located at specific locations in zones and the booths may include objects that are on display. Certain locations may be more prone to foot traffic and/or more likely for people to attend due to their proximity to certain other objects (e.g., bathrooms, food courts, entrances, exits, other popular booths, etc.). In some instances, certain locations may be more likely for people to attend based on the layout of the physical space and/or the way the other booths are arranged in the physical space.

It may be desirable to determine which people at an event (e.g., convention, art show, vehicle show, etc.) attend certain booths in certain zones. For example, it may be beneficial to determine the paths of people that have authority to make decisions for a company (e.g., “C” level employees (e.g., chief executive officer, chief sales officer, chief financial officer, chief operations officer, etc.)). It may be desirable to determine the paths of the people in the physical space to better understand which zones including booths are attended and which ones are not attended. It may be desirable to understand the amounts of time that certain people attend certain booths in certain zones. The path analytics may enable determining where to locate certain booths in order to increase attendance at the booths and/or decrease attendance at the booths. For example, certain vendors may pay a fee to increase their chances of their booths being attended more. To that end, it may be beneficial to determine the paths of people and which locations in a physical space are more likely to be attended to enable recommending to place certain booths at certain locations in the physical space.

To enable path analytics, some embodiments of the present disclosure may utilize smart floor tiles that are disposed in a physical space where people may move around. For example, the smart floor tiles may be installed in a floor of a convention hall where vendors display objects at booths in certain zones. The smart floor tiles may be capable of measuring data (e.g., pressure) associated with footsteps of the people and transmitting the measured data to a cloud-based computing system that analyzes the measured data. In some embodiments, moulding sections and/or a camera may be used to measure the data and/or supplement the data measured by the smart floor tiles. The accuracy of the measurements pertaining to the path of the people may be improved using the smart floor tiles as they measure the physical pressure of the footsteps of the person to track the path of the person and/or other gait characteristics (e.g., width of feet, speed of gait, amount of time spent at certain locations, etc.).

Further, the paths of the people may be correlated with other information, such as job titles of the people, age of the people, gender of the people, employers of the people, and the like. This information may be retrieved from a third party data source and/or data source internal to the cloud-based computing system. For example, the cloud-based computing system may be communicatively coupled with one or more web services (e.g., application programming interfaces) that provide the information to the cloud-based computing system.

The paths that are generated for the people may be overlaid on a virtual representation of the physical space including and/or excluding graphics representing the zones, booths located in the zones, and/or objects displayed in the booths in the physical space. All of the paths of all of the people that move around the physical space during an event, for example, may be overlaid on each other on a user interface presented on a computing device. In some embodiments, a user may select to filter the paths that are presented to just paths of people having a certain job title, to a longest path, to paths that indicate the people visited certain booths, to paths that spent a certain amount of time at a particular zone and/or booth, and the like. The filtering may be performed using any suitable criteria. Accordingly, the disclosed techniques may improve the user's experience using a computing device because an improved user interface that presents desired paths may be provided to the user such that path analytics are enhanced.

The enhanced path analytics may enable the user to make a better determination regarding the layout of booths and/or zones. Further, in some embodiments, the cloud-based computing system may analyze the paths and provide recommendations for locating objects in the physical space. For example, if a certain object has a certain priority and the cloud-based computing system determines a certain zone is the most highly attended zone, then the cloud-based computing system may recommend to move the certain object to that certain zone to increase the likelihood that the object will be seen by people.

Barring unforeseeable changes in human locomotion, humans can be expected to generate measurable interactions with buildings through their footsteps on buildings' floors. In some embodiments the smart floor tiles may help realize the potential of a “smart building” by providing, amongst other things, control inputs for a building's environmental control systems using directional occupancy sensing based on occupants' interaction with building surfaces, including, without limitation, floors, and/or interaction with a physical space including their location relative to moulding sections.

The moulding sections, may include a crown moulding, a baseboard, a shoe moulding, a door casing, and/or a window casing, that are located around a perimeter of a physical space. The moulding sections may be modular in nature in that the moulding sections may be various different sizes and the moulding sections may be connected with moulding connectors. The moulding connectors may be configured to maintain conductivity between the connected moulding sections. To that end, each moulding section may include various components, such as electrical conductors, sensors, processors, memories, network interfaces, and so forth that enable communicating data, distributing power, obtaining moulding section sensor data, and so forth. The moulding sections may use various sensors to obtain moulding section sensor data including the location of objects in a physical space as the objects move around the physical space. The moulding sections may use moulding section sensor data to determine a path of the object in the physical space and/or to control other electronic devices (e.g., smart shades, smart windows, smart doors, HVAC system, smart lights, and so forth) in the smart building. Accordingly, the moulding sections may be in wired and/or wireless communication with the other electronic devices. Further, the moulding sections may be in electrical communication with a power supply. The moulding sections may be powered by the power supply and may distribute power to smart floor tiles that may also be in electrical communication with the moulding sections.

A camera may provide a livestream of video data and/or image data to the cloud-based computing system. The data from the camera may be used to identify certain people in a room and/or track the path of the people in the room. Further, the data may be used to monitor one or more parameters pertaining to a gait of the person to aid in the path analytics. For example, facial recognition may be performed using the data from the camera to identify a person when they first enter a physical space and correlate the identity of the person with the person's path when the person begins to walk on the smart floor tiles.

The cloud-based computing system may monitor one or more parameters of the person based on the measured data from the smart floor tiles, the moulding sections, and/or the camera. The one or more parameters may be associated with the gait of the person and/or the path of the person. Based on the one or more parameters, the cloud-based computing system may determine paths of people in the physical space. The cloud-based computing system may perform any suitable analysis of the paths of the people.

In addition, there are a multitude of scenarios where it may be beneficial to perform an action based on a location of a person in a physical space. Example scenarios may include (i) preventing a person having a particular medical condition (e.g., neurodegenerative disease) from leaving a nursing home or their room in the nursing home by locking a door, (ii) enabling a person to exit a building (e.g., during an emergency, such as fire, attack, flood, etc.) by unlocking a door and/or window and/or opening the door and/or window, (iii) preventing a hostile person from entering a particular room by locking a door and/or window and/or closing a door and/or window, and so forth. However, accurately determining the location of a person in a physical space may be technically difficult for a computing system that is located distally from the physical space in which the person is located. Further, causing a device (e.g., an actuation mechanism) to effectively perform an action from a distal location may be a technically challenging problem.

Accordingly, some of the disclosed embodiments provide a technical solution to such technical challenges by using one or more smart floor tiles, moulding sections, and/or cameras to enable a cloud-based computing system to accurately determine a location of a person in a physical space. The cloud-based computing system may determine a distance from the location of the person to a location of an object. In some embodiments, prior to determining the distance from the location of the person to the location of the object, the cloud-based computing device may determine an identity of the person. The cloud-based computing system may use a list of people that are to be monitored (e.g., a watch list of patients in a nursing home, a list of criminal offenders, etc.). In some embodiments, the cloud-based computing system may determine the distance only if the identity of the person is found in the list. In some embodiments, for example, when there is an emergency (e.g., a fire), the cloud-based computing system may not check the list prior to determining the distance of the location of the person from the location of the object.

Each of the scenarios described above may be aided efficiently, accurately, and beneficially by the disclosed techniques to increase the quality of individual lives and/or society. The cloud-based computing system may be communicatively coupled to one or more devices. In response to determining the location of the person is within a threshold distance from the location of the object, the cloud-based computing system may transmit a control signal to the one or more devices to cause the one or more devices to perform an action.

For example, in some embodiments, the object may be a door or a window, the device may be an actuation mechanism (e.g. a lock, an electromechanical arm, etc.), and the control signal may cause the actuation mechanism to actuate. In one example, when a person having a certain medical condition approaches a door within a certain threshold distance, the disclosed techniques may be used to cause the actuation mechanism to lock the door, or to close and lock the door (e.g., using both the electromechanical arm and the lock), to prevent the person from leaving their patient room or a nursing home. In other instances, if there is an emergency situation, such as a fire in a building, and the cloud-based computing system detects (e.g., via data from the smart floor tiles, moulding sections, and/or cameras) a person is trapped in a particular room having a locked window, then the disclosed techniques may be used to cause the actuation mechanism to unlock the window and/or open the window to enable the person to exit through the window.

Further, after the location of the person is determined and the action has been performed by the device, the path of the person may be monitored. For example, if the person walks to another room in the physical space and approaches another object, the disclosed techniques may be used to cause another device (e.g., another lock) to perform an action (e.g., actuate to lock the door). In such a way, the disclosed embodiments may continuously monitor the location and path of the person in the physical space to cause actions to be performed to enhance the safety and/or wellbeing of the patient and/or other people.

In some embodiments, the device may be a computing device of the patient and/or a medical personnel (e.g., nurse), and the control signal may cause the device to present a notification including information. The information may pertain to the patient (e.g., name, age, gender, medical conditions, etc.), the location of the patient, and so forth. The notification may instruct the patient to return to another location. The notification may instruct the medical personnel that the patient is wandering around and about to leave the physical space, and further to track down the patient and/or escort the patient back to another location. Such techniques may enhance the safety and/or wellbeing of the patient and/or other people.

Turning now to the figures, FIGS. 2000A-2000E illustrate various example configurations of components of a system 10 according to certain embodiments of this disclosure. FIG. 2000A visually depicts components of the system in a first room 21.5 and a second room 23.5 and FIG. 2000B depicts a high-level component diagram of the system 10.5. For purposes of clarity, FIGS. 2000A and 2000B are discussed together below.

The first room 21.5, in this example, is a convention hall room in a convention center where a person 25.5 is attending an event. However, the first room 21.5 may be any suitable room that includes a floor capable of being equipped with smart floor tiles 112.5, moulding sections 102.5, and/or a camera 50.5. The second room 23.5, in this example, is an entry station in the care convention center.

When the person initially arrives to the convention center, the person 25.15 may check in and/or register for the event being held in the first room 21.5. As depicted, the person may carry a computing device 12.5, which may be a smartphone, a laptop, a tablet, a pager, a card, or any suitable computing device. The person 25.15 may use the computing device 12.5 to check in to the event. For example, the person may 25.15 may swipe the computing device 12.5 or place it next to a reader that extracts data and sends the data to the cloud-based computing system 116.5. The data may include an identity of the person 25.15. The reception of the data at the cloud-based computing system 116.5 may be referred to as an initiation event of a path of an object (e.g., person 25.15) in the physical space (e.g., first room 21.5) at a first time in a time series. In some embodiments, a camera 50.5 may send data to the cloud-based computing system 116.5 that performs facial recognition techniques to determine the identity of the person 25.15. Receiving the data from the camera 50.5 may also be referred to as an initiation event herein.

Subsequently to the initiation event occurring, the cloud-based computing system 116.5 may receive data from a first smart floor tile 112.5 that the person 25.25 steps on at a second time (subsequent to the first time in the time series). The data from the first smart floor tile 112.5 may occur at a location event that includes an initial location of the person in the physical space. The cloud-based computing device may correlate the initiation event and the initial location to generate a starting point of a path of the person 25.25 in the first room 21.5.

The person 25.35 may walk around the first room 21.5 to visit a booth 27.5. The smart floor tiles 112.5 may be continuously or continually transmitting measurement data to the cloud-based computing system 116.5 as the person 25.35 walks from the entrance of the first room 21.5 to the booth 27.5. The cloud-based computing system 116.5 may generate a path 31.5 of the person 25.35 through the first room 21.5.

The first room 21.5 may also include at least one electronic device 13.5, which may be any suitable electronic device, such as a smart thermostat, smart vacuum, smart light, smart speaker, smart electrical outlet, smart hub, smart appliance, smart television, etc.

Each of the smart floor tiles 112.5, moulding sections 102.5, camera 50.5, computing device 12.5, and/or electronic device 13.5 may be capable of communicating, either wirelessly and/or wired, with the cloud-based computing system 116.5 via a network 20.5. As used herein, a cloud-based computing system refers, without limitation, to any remote or distal computing system accessed over a network link. Each of the smart floor tiles 112.5, moulding sections 102.5, camera 50.5, computing device 12.5, and/or electronic device 13.5 may include one or more processing devices, memory devices, and/or network interface devices.

The network interface devices of the smart floor tiles 112.5, moulding sections 102.5, camera 50.5, computing device 12.5, and/or electronic device 13.5 may enable communication via a wireless protocol for transmitting data over short distances, such as Bluetooth, ZigBee, near field communication (NFC), etc. Additionally, the network interface devices may enable communicating data over long distances, and in one example, the smart floor tiles 112.5, moulding sections 102.5, camera 50.5, computing device 12.5, and/or electronic device 13.5 may communicate with the network 20.5. Network 20.5 may be a public network (e.g., connected to the Internet via wired (Ethernet) or wireless (WiFi)), a private network (e.g., a local area network (LAN), wide area network (WAN), virtual private network (VPN)), or a combination thereof.

The computing device 12.5 may be any suitable computing device, such as a laptop, tablet, smartphone, or computer. The computing device 12.5 may include a display that is capable of presenting a user interface. The user interface may be implemented in computer instructions stored on a memory of the computing device 12.5 and/or computing device 15 and executed by a processing device of the computing device 12.5. The user interface may be a stand-alone application that is installed on the computing device 12.5 or may be an application (e.g., website) that executes via a web browser.

The user interface may be generated by the cloud-based computing system 116.5 and may present various paths of people in the first room 21.5 on the display screen. The user interface may include various options to filter the paths of the people based on criteria. Also, the user interface may present recommended locations for certain objects in the first room 21.5. The user interface may be presented on any suitable computing device. For example, computing device 15.5 may receive and present the user interface to a person interested in the path analytics provided using the disclosed embodiments. The computing device 15.5 may be any suitable computing device, such as a laptop, tablet, smartphone, or computer.

In some embodiments, the cloud-based computing system 116.5 may include one or more servers 128.5 that form a distributed, grid, and/or peer-to-peer (P2P) computing architecture. Each of the servers 128.5 may include one or more processing devices, memory devices, data storage, and/or network interface devices. The servers 128.5 may be in communication with one another via any suitable communication protocol. The servers 128.5 may receive data from the smart floor tiles 112.5, moulding sections 102.5, and/or the camera 50.5 and monitor a parameter pertaining to a gait of the person 25.5 based on the data. For example, the data may include pressure measurements obtained by a sensing device in the smart floor tile 112.5. The pressure measurements may be used to accurately track footsteps of the person 25.5, walking paths of the person 25.5, gait characteristics of the person 25.5, walking patterns of the person 25.5 throughout each day, and the like. The servers 128.5 may determine an amount of gait deterioration based on the parameter. The servers 128.5 may determine whether a propensity for a fall event for the person 25.5 satisfies a threshold propensity condition based on (i) the amount of gait deterioration satisfying a threshold deterioration condition, or (ii) the amount of gait deterioration satisfying the threshold deterioration condition within a threshold time period. If the propensity for the fall event for the person 25.5 satisfies the threshold propensity condition, the servers 128.5 may select one or more interventions to perform for the person 25.5 to prevent the fall event from occurring and may perform the one or more selected interventions. The servers 128.5 may use one or more machine learning models 154.5 trained to monitor the parameter pertaining to the gait of the person 25.5 based on the data, determine the amount of gait deterioration based on the parameter, and/or determine whether the propensity for the fall event for the person satisfies the threshold propensity condition.

In some embodiments, the cloud-based computing system 116.5 may include a training engine 152.5 and/or the one or more machine learning models 154.5. The training engine 152.5 and/or the one or more machine learning models 154.5 may be communicatively coupled to the servers 128.5 or may be included in one of the servers 128.5. In some embodiments, the training engine 152.5 and/or the machine learning models 154.5 may be included in the computing device 12.5, computing device 15.5, and/or electronic device 13.5.

The one or more of machine learning models 154.5 may refer to model artifacts created by the training engine 152.5 using training data that includes training inputs and corresponding target outputs (correct answers for respective training inputs). The training engine 152.5 may find patterns in the training data that map the training input to the target output (the answer to be predicted), and provide the machine learning models 154.5 that capture these patterns. The set of machine learning models 154.5 may comprise, e.g., a single level of linear or non-linear operations (e.g., a support vector machine [SVM]) or a deep network, i.e., a machine learning model comprising multiple levels of non-linear operations. Examples of such deep networks are neural networks including, without limitation, convolutional neural networks, recurrent neural networks with one or more hidden layers, and/or fully connected neural networks.

In some embodiments, the training data may include inputs of parameters, variations in the parameters, variations in the parameters within a threshold time period, or some combination thereof and correlated outputs of locations of objects to be placed in the first room 21.5 based on the parameters. That is, in some embodiments, there may be a separate respective machine learning model 154.5 for each individual parameter that is monitored. The respective machine learning model 154.5 may output a recommended location for an object based on the parameters (e.g., amount of time people spend at certain locations, paths of people, etc.).

In some embodiments, the cloud-based computing system 116.5 may include a database 129.5. The database 129.5 may store data pertaining to paths of people (e.g., a visual representation of the path, identifiers of the smart floor tiles 112.5 the person walked on, the amount of time the person stands on each smart floor tile 112.5 (which may be used to determine an amount of time the person spends at certain booths), and the like), identities of people, job titles of people, employers of people, age of people, gender of people, residential information of people, and the like. In some embodiments, the database 129.5 may store data generated by the machine learning models 154.5, such as recommended locations for objects in the first room 21.5. Further, the database 129.5 may store information pertaining to the first room 21.5, such as the type and location of objects displayed in the first room 21.5, the booths included in the first room 21.5, the zones (e.g., boundaries) including the booths including the objects in the first room, the vendors that are hosting the booths, and the like. The database 129.5 may also store information pertaining to the smart floor tile 112.5, moulding section 102.5, and/or the camera 50.5, such as device identifiers, addresses, locations, and the like. The database 129.5 may store paths for people that are correlated with an identity of the person 25.5. The database 129.5 may store a map of the first room 21.5 including the smart floor tiles 112.5, moulding sections 102.5, camera 50.5, any booths 27.5, and so forth. The database 129.5 may store video data of the first room 21.5. The training data used to train the machine learning models 154.5 may be stored in the database 129.5.

The camera 50.5 may be any suitable camera capable of obtaining data including video and/or images and transmitting the video and/or images to the cloud-based computing system 116.5 via the network 20.5. The data obtained by the camera 50.5 may include timestamps for the video and/or images. In some embodiments, the cloud-based computing system 116.5 may perform computer vision to extract high-dimensional digital data from the data received from the camera 50.5 and produce numerical or symbolic information. The numerical or symbolic information may represent the parameters monitored pertaining to the path of the person 25.5 monitored by the cloud-based computing system 116.5. The video data obtained by the camera 50.5 may be used for facial recognition of the person 25.5.

FIGS. 2000C-2000E depict various example configurations of smart floor tiles 112.5, and/or moulding sections 102.5 according to certain embodiments of this disclosure. FIG. 2000C depicts an example system 10.5 that is used in a physical space of a smart building (e.g., care facility). The depicted physical space includes a wall 104.5, a ceiling 106.5, and a floor 108.5 that define a room. Numerous moulding sections 102A.5, 102B.5, 102C.5, and 102D.5 are disposed in the physical space. For example, moulding sections 102A.5 and 102B.5 may form a baseboard or shoe moulding that is secured to the wall 108.5 and/or the floor 108.5. Moulding sections 102C.5 and 102D.5 may for a crown moulding that is secured to the wall 108.5 and/or the ceiling 106.5. Each moulding section 102A.5 may have different shapes and/or sizes.

The moulding sections 102 may each include various components, such as electrical conductors, sensors, processors, memories, network interfaces, and so forth. The electrical conductors may be partially or wholly enclosed within one or more of the moulding sections. For example, one electrical conductor may be a communication cable that is partially enclosed within the moulding section and exposed externally to the moulding section to electrically couple with another electrical conductor in the wall 108.5. In some embodiments, the electrical conductor may be communicably connected to at least one smart floor tile 112.5. In some embodiments, the electrical conductor may be in electrical communication with a power supply 114.5. In some embodiments, the power supply 114.5 may provide electrical power that is in the form of mains electricity general-purpose alternating current. In some embodiments, the power supply 114.5 may be a battery, a generator, or the like.

In some embodiments, the electrical conductor is configured for wired data transmission. To that end, in some embodiments the electrical conductor may be communicably coupled via cable 118.5 to a central communication device 120.5 (e.g., a hub, a modem, a router, etc.). Central communication device 120 may create a network, such as a wide area network, a local area network, or the like. Other electronic devices 13.5 may be in wired and/or wireless communication with the central communication device 120.5. Accordingly, the moulding section 102.5 may transmit data to the central communication device 120.5 to transmit to the electronic devices 13.5. The data may be control instructions that cause, for example, an the electronic device 13.5 to change a property. In some embodiments, the moulding section 102A.5 may be in wired and/or wireless communication connection with the electronic device 13.5 without the use of the central communication device 120.5 via a network interface and/or cable. The electronic device 13.5 may be any suitable electronic device capable of changing an operational parameter in response to a control instruction.

In some embodiments, the electrical conductor may include an insulated electrical wiring assembly. In some embodiments, the electrical conductor may include a communications cable assembly. The moulding sections 102.5 may include a flame-retardant backing layer. The moulding sections 102.5 may be constructed using one or more materials selected from: wood, vinyl, rubber, fiberboard, metal, plastic, and wood composite materials.

The moulding sections may be connected via one or more moulding connectors 110.5. A moulding connector 110.5 may enhance electrical conductivity between two moulding sections 102.5 by maintaining the conductivity between the electrical conductors of the two moulding sections 102.5. For example, the moulding connector 110.5 may include contacts and its own electrical conductor that forms a closed circuit when the two moulding sections are connected with the moulding connector 110.5. In some embodiments, the moulding connectors 110.5 may include a fiber optic relay to enhance the transfer of data between the moulding sections 102.5. It should be appreciated that the moulding sections 102.5 are modular and may be cut into any desired size to fit the dimensions of a perimeter of a physical space. The various sized portions of the moulding sections 102.5 may be connected with the moulding connectors 110.5 to maintain conductivity.

Moulding sections 102.5 may utilize a variety of sensing technologies, such as proximity sensors, optical sensors, membrane switches, pressure sensors, and/or capacitive sensors, to identify instances of an object proximate or located near the sensors in the moulding sections and to obtain data pertaining to a gait of the person 25.5. Proximity sensors may emit an electromagnetic field or a beam of electromagnetic radiation (infrared, for instance), and identify changes in the field or return signal. The object being sensed may be any suitable object, such as a human, an animal, a robot, furniture, appliances, and the like. Sensing devices in the moulding section may generate moulding section sensor data indicative of gait characteristics of the person 25.5, location (presence) of the person 25.5, the timestamp associated with the location of the person 25.5, and so forth.

The moulding section sensor data may be used alone or in combination with tile impression data generated by the smart floor tiles 112.5 and/or image data generated by the camera 50.5 to perform path analytics for people. For example, the moulding section sensor data may be used to determine a control instruction to generate and to transmit to an electric device 13.5 and/or the smart floor tile 102A.5. The control instruction may include changing an operational parameter of the electronic device 13.5 based on the moulding section sensor data. The control instruction may include instructing the smart floor tile 112.5 to reset one or more components based on an indication in the moulding section sensor data that the one or more components is malfunctioning and/or producing faulty results. Further, the moulding sections 102.5 may include a directional indicator (e.g., light) that emits different colors of light, intensities of light, patterns of light, etc. based on path analytics of the cloud-based computing system 116.5.

In some embodiments, the moulding section sensor data can be used to verify the impression tile data and/or image data of the camera 50.5 is accurate for generating and analyzing paths of people. Such a technique may improve accuracy of the path analytics. Further, if the moulding section sensor data, the impression tile data, and/or the image data do not align (e.g., the moulding section sensor data does not indicate a path of a person and impression tile data indicates a path of the person), then further analysis may be performed. For example, tests can be performed to determine if there are defective sensors at the corresponding smart floor tile 112.5 and/or the corresponding moulding section 102 that generated the data. Further, control actions may be performed such as resetting one or more components of the moulding section 102.5 and/or the smart floor tile 112.5. In some embodiments, preference to certain data may be made by the cloud-based computing system 116.5. For example, in one embodiment, preference for the impression tile data may be made over the moulding section sensor data and/or the image data, such that if the impression tile data differs from the moudling section sensor data and/or the image data, the impression tile data is used to perform path analytics.

FIG. 2000D illustrates another configuration of the moulding sections 102.5. In this example, the moulding sections 102E.5-102H.5 surround a border of a smart window 155.5. The moulding sections 102.5 are connected via the moulding connector 110.5. As may be appreciated, the modular nature of the moulding sections 102.5 with the moulding connectors 110.5 enables forming a square around the window. Other shapes may be formed using the moulding sections 102.5 and the moulding connectors 110.5.

The moulding sections 102.5 may be electrically and/or communicably connected to the smart window 155.5 via electrical conductors and/or interfaces. The moulding sections 102.5 may provide power to the smart window 155.5, receive data from the smart window 155.5, and/or transmit data to the smart window 155.5. One example smart window includes the ability to change light properties using voltage that may be provided by the moulding sections 102.5. The moulding sections 102.5 may provide the voltage to control the amount of light let into a room based on path analytics. For example, if the moulding section sensor data, impression tile data, and/or image data indicates a portion of the first room 21.5 includes a lot of people, the cloud-based computing system 116.5 may perform an action by causing the moulding sections 102.5 to instruct the smart window 155.5 to change a light property to allow light into the room. In some instances the cloud-based computing system 116.5 may communicate directly with the smart window 155.5 (e.g., electronic device 13.5).

In some embodiments, the moulding sections 102.5 may use sensors to detect when the smart window 155.5 is opened. The moulding sections 102.5 may determine whether the smart window 155.5 opening is performed at an expected time (e.g., when a home owner is at home) or at an unexpected time (e.g., when the home owner is away from home). The moulding sections 102.5, the camera 50.5, and/or the smart floor tile 112.5 may sense the occupancy patterns of certain objects (e.g., people) in the space in which the moulding sections 102.5 are disposed to determine a schedule of the objects. The schedule may be referenced when determining if an undesired opening (e.g., break-in event) occurs and the moulding sections 102.5 may be communicatively to an alarm system to trigger the alarm when the certain event occurs.

The schedule may also be referenced when determining a medical condition of the person 25.5. For example, if the schedule indicates that the person 25.5 went to the bathroom a certain number of times (e.g., 10) within a certain time period (e.g., 1 hour), the cloud-based computing system 116.5 may determine that the person has a urinary tract infection (UTI) and may perform an intervention, such as transmitting a message to the computing device 12.5 of the person 25.5. The message may indicate the potential UTI and recommend that the person 25.5 schedules an appointment with a medical personnel.

As depicted, at least moulding section 102F.5 is electrically and/or communicably coupled to smart shades 160.5. Again, the cloud-based computing system 116.5 may cause the moulding section 102F.5 to control the smart shades 160.5 to extend or retract to control the amount of light let into a room. In some embodiments, the cloud-based computing system 116.5 may communicate directly with the smart shades 160.5.

FIG. 2000E illustrates another configuration of the moulding sections 102.5 and smart floor tiles 112.5. In this example, the moulding sections 102E.5-102H.5 surround a majority of a border of a smart door 170.5. The moulding sections 102J.5, 102K.5, and 102L.5 and/or the smart floor tile 112.5 may be electrically and/or communicably connected to the smart door 170.5 via electrical conductors and/or interfaces. The moulding sections 102.5 and/or smart floor tiles 112.5 may provide power to the smart door 170.5, receive data from the smart door 170.5, and/or transmit data to the smart door 170.5. In some embodiments, the moulding sections 102.5 and/or smart floor tiles 112.5 may control operation of the smart door 170.5. For example, if the moulding section sensor data and/or impression tile data indicates that no one is present in a house for a certain period of time, the moulding sections 102.5 and/or smart floor tiles 112.5 may determine a locked state of the smart door 170.5 and generate and transmit a control instruction to the smart door 170.5 to lock the smart door 170.5 if the smart door 170.5 is in an unlocked state.

In another example, the moulding section sensor data, impression tile data, and/or the image data may be used to generate gait profiles for people in a smart building (e.g., care facility). When a certain person is in the room near the smart door 170.5, the cloud-based computing device 116.5 may detect that person's presence based on the data received from the smart floor tiles, moulding sections 102.5, and/or camera 50.5. In some embodiments, if the person 25.5 is detected near the smart door 170.5, the cloud-based computing system 116.5 may determine whether the person 25.5 has a particular medical condition (e.g., alzheimers) and/or a flag is set that the person should not be allowed to leave the smart building. If the person is detected near the smart door 170.5 and the person 25.5 has the particular medical condition and/or the flag set, then the cloud-based computing system 116.5 may cause the moulding sections 102.5 and/or smart floor tiles 112.5 to control the smart door 170.5 to lock the smart door 170.5. In some embodiments, the cloud-based computing system 116.5 may communicate directly with the smart door 170.5 to cause the smart door 170.5 to lock.

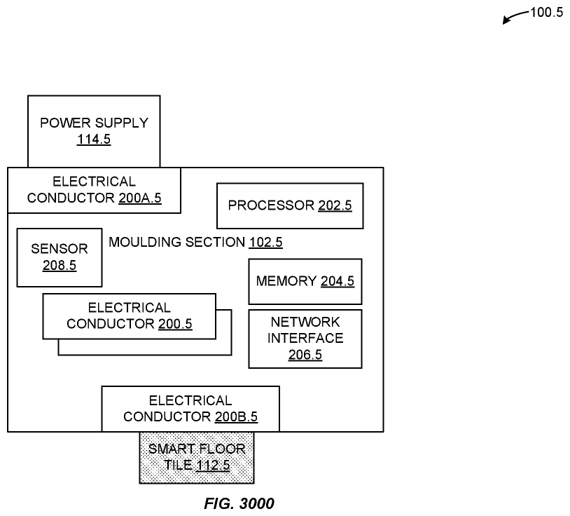

FIG. 3000 illustrates an example component diagram of a moulding section 102.5 according to certain embodiments of this disclosure. As depicted, the moulding section 102 includes numerous electrical conductors 200.5, a processor 202.5, a memory 204.5, a network interface 206.5, and a sensor 208.5. More or fewer components may be included in the moulding section 102.5. The electrical conductors may be insulated electrical wiring assemblies, communications cable assemblies, power supply assemblies, and so forth. As depicted, one electrical conductor 200A.5 may be in electrical communication with the power supply 114.5, and another electrical conductor 200B.5 may be communicably connected to at least one smart floor tile 112.5.

In various embodiments, the moulding section 102.5 further comprises a processor 202.5. In the non-limiting example shown in FIG. 3000, processor 202.5 is a low-energy microcontroller, such as the ATMEGA328P by Atmel Corporation. According to other embodiments, processor 202.5 is the processor provided in other processing platforms, such as the processors provided by tablets, notebook or server computers.

In the non-limiting example shown in FIG. 3000, the moulding section 102.5 includes a memory 204.5. According to certain embodiments, memory 204.5 is a non-transitory memory containing program code to implement, for example, generation and transmission of control instructions, networking functionality, the algorithms for generating and analyzing locations, presence, paths, and/or tracks, and the algorithms for performing path analytics as described herein.

Additionally, according to certain embodiments, the moulding section 102.5 includes the network interface 206.5, which supports communication between the moulding section 102.5 and other devices in a network context in which smart building control using directional occupancy sensing and path analytics is being implemented according to embodiments of this disclosure. In the non-limiting example shown in FIG. 3000, network interface 206.5 includes circuitry for sending and receiving data using Wi-Fi, including, without limitation at 900 MHz, 2.8 GHz and 5.0 GHz. Additionally, network interface 206.5 includes circuitry, such as Ethernet circuitry for sending and receiving data (for example, smart floor tile data) over a wired connection. In some embodiments, network interface 206.5 further comprises circuitry for sending and receiving data using other wired or wireless communication protocols, such as Bluetooth Low Energy or Zigbee circuitry. The network interface 206.5 may enable communicating with the cloud-based computing device 116.5 via the network 20.5.

Additionally, according to certain embodiments, network interface 206.5 which operates to interconnect the moulding device 102.5 with one or more networks. Network interface 206.5 may, depending on embodiments, have a network address expressed as a node ID, a port number or an IP address. According to certain embodiments, network interface 206.5 is implemented as hardware, such as by a network interface card (NIC). Alternatively, network interface 206.5 may be implemented as software, such as by an instance of the java.net.NetworkInterface class. Additionally, according to some embodiments, network interface 206.5 supports communications over multiple protocols, such as TCP/IP as well as wireless protocols, such as 3G or Bluetooth. Network interface 206.5 may be in communication with the cloud-based computing system 116.5.

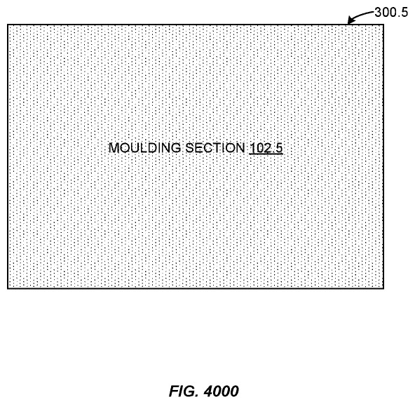

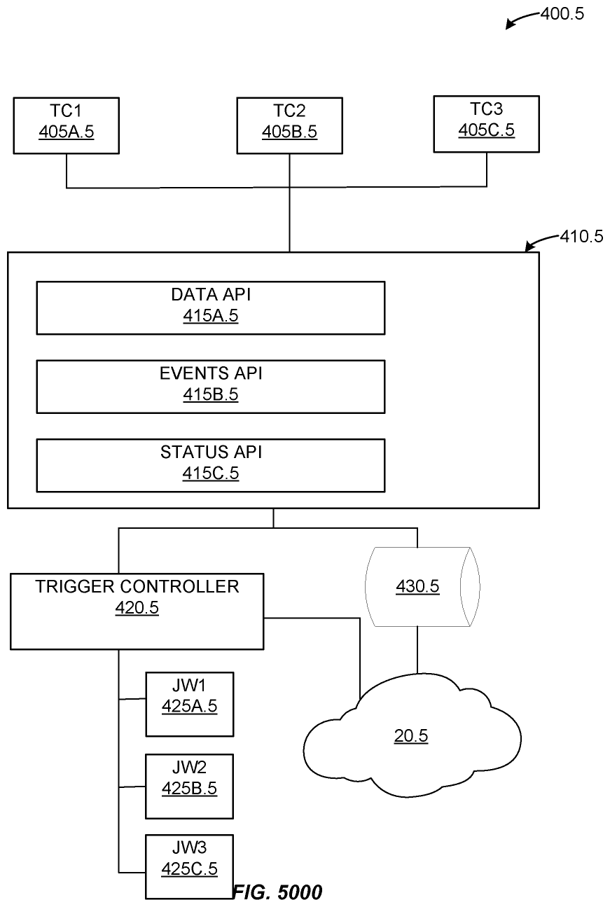

FIG. 4000 illustrates an example backside view 300.5 of a moulding section 102.5 according to certain embodiments of this disclosure. As depicted by the dots 300.5, the backside of the moulding section 102.5 may include a fire-retardant backing layer positioned between the moulding section 102.5 and the wall to which the moulding section 102.5 is secured.

FIG. 5000 illustrates a network and processing context 400.5 for smart building control using directional occupancy sensing and path analytics according to certain embodiments of this disclosure. The embodiment of the network context 400.5 shown in FIG. 5000 is for illustration only and other embodiments could be used without departing from the scope of the present disclosure.

In the non-limiting example shown in FIG. 5000, a network context 400.5 includes one or more tile controllers 405A.5, 405B.5 and 405C.5, an API suite 410.5, a trigger controller 420.5, job workers 425A.5-425C.5, a database 430.5 and a network 435.5.

According to certain embodiments, each of tile controllers 405A.5-405C.5 is connected to a smart floor tile 112.5 in a physical space. Tile controllers 405A.5-405C.5 generate floor contact data (also referred to as impression tile data herein) from smart floor tiles in a physical space and transmit the generated floor contact data to API suite 410.5. In some embodiments, data from tile controllers 405A.5-405C.5 is provided to API suite 410.5 as a continuous stream. In the non-limiting example shown in FIG. 5000, tile controllers 405A.5-405C.5 provide the generated floor contact data from the smart floor tile to API suite 410.5 via the internet. Other embodiments, wherein tile controllers 405A.5-405C.5 employ other mechanisms, such as a bus or Ethernet connection to provide the generated floor data to API suite 410.5 are possible and within the intended scope of this disclosure.

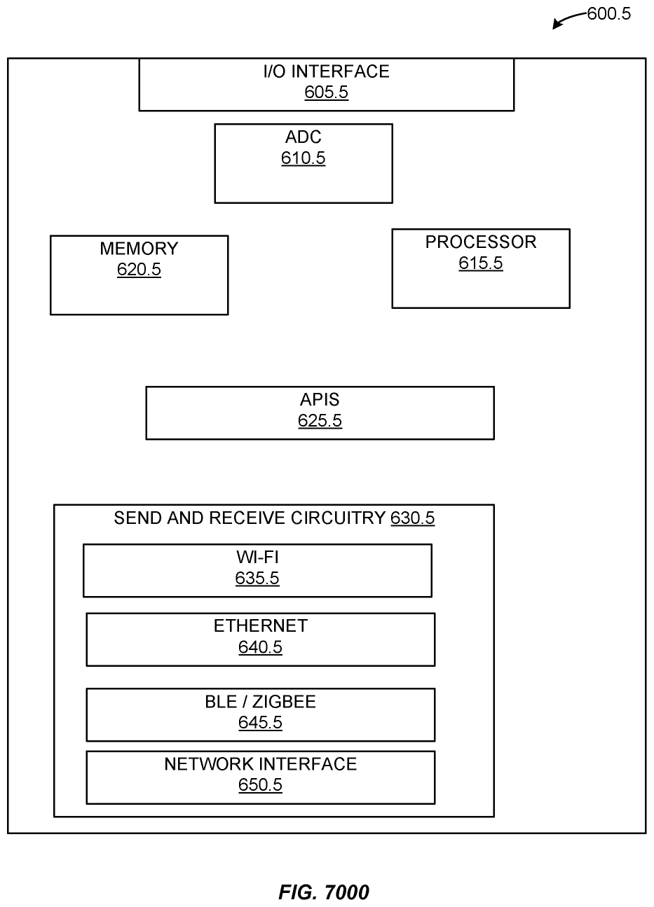

According to some embodiments, API suite 410.5 is embodied on a server 128.5 in the cloud-based computing system 116.5 connected via the internet to each of tile controllers 405A.5-405C.5. According to some embodiments, API suite is embodied on a master control device, such as master control device 600.5 shown in FIG. 7000 of this disclosure. In the non-limiting example shown in FIG. 5000, API suite 410.5 comprises a Data Application Programming Interface (API) 415A.5, an Events API 415B.5 and a Status API 215C.5.

In some embodiments, Data API 415A.5 is an API for receiving and recording tile data from each of tile controllers 405A.5-405C.5. Tile events include, for example, raw, or minimally processed data from the tile controllers, such as the time and data a particular smart floor tile was pressed and the duration of the period during which the smart floor tile was pressed. According to certain embodiments, Data API 415A.5 stores the received tile events in a database such as database 430.5. In the non-limiting example shown in FIG. 5000, some or all of the tile events are received by API suite 410.5 as a stream of event data from tile controllers 405A.5-405C.5, Data API 415A.5 operates in conjunction with trigger controller 420.5 to generate and pass along triggers breaking the stream of tile event data into discrete portions for further analysis.

According to various embodiments, Events API 415B.5 receives data from tile controllers 405A.5-405C.5 and generates lower-level records of instantaneous contacts where a sensor of the smart floor tile is pressed and released.

In the non-limiting example shown in FIG. 5000, Status API 415C.5 receives data from each of tile controllers 405A.5-405C.5 and generates records of the operational health (for example, CPU and memory usage, processor temperature, whether all of the sensors from which a tile controller receives inputs is operational) of each of tile controllers 405A.5-405C.5. According to certain embodiment, status API 415C.5 stores the generated records of the tile controllers' operational health in database 430.5.

According to some embodiments, trigger controller 420.5 operates to orchestrate the processing and analysis of data received from tile controllers 405A.5-405C.5. In addition to working with data API 415A.5 to define and set boundaries in the data stream from tile controllers 405A.5-405C.5 to break the received data stream into tractably sized and logically defined “chunks” for processing, trigger controller 420 also sends triggers to job workers 425A.5-425C.5 to perform processing and analysis tasks. The triggers comprise identifiers uniquely identifying each data processing job to be assigned to a job worker. In the non-limiting example shown in FIG. 5000, the identifiers comprise: 1.) a sensor identifier (or an identifier otherwise uniquely identifying the location of contact); 2.) a time boundary start identifying a time in which the smart floor tile went from an idle state (for example, an completely open circuit, or, in the case of certain resistive sensors, a baseline or quiescent current level) to an active state (a closed circuit, or a current greater than the baseline or quiescent level); and 3.) a time boundary end defining the time in which a smart floor tile returned to the idle state.

In some embodiments, each of job workers 425A.5-425C.5 corresponds to an instance of a process performed at a computing platform, (for example, cloud-based computing system 116.5 in FIG. 2000A) for determining paths and performing an analysis of the paths (e.g., such as filtering paths based on criteria, recommending a location of an object based on the paths, predicting a propensity for a fall event and performing an intervention based on the propensity). Instances of processes may be added or subtracted depending on the number of events or possible events received by API suite 410.5 as part of the data stream from tile controllers 405A.5-205C.5. According to certain embodiments, job workers 425A.5-425C.5 perform an analysis of the data received from tile controllers 405A.5-405C.5, the analysis having, in some embodiments, two stages. A first stage comprises deriving footsteps, and paths, or tracks, from impression tile data. A second stage comprises characterizing those footsteps, and paths, or tracks, to determine gait characteristics of the person 25.5. The paths and/or gait characteristics may be presented to an online dashboard (in some embodiments, provided by a UI on an electronic device, such as computing device 12.5 or 15.5 in FIG. 2000A) and to generate control signals for devices (e.g., the computing devices 12.5 and/or 15.5, the electronic device 15.5, the moulding sections 102.5, the camera 50.5, and/or the smart floor tile 112.5 in FIG. 2000A) controlling operational parameters of a physical space where the smart floor impression tile data were recorded.

In the non-limiting example shown in FIG. 5000, job workers 425A.5-425C.5 perform the constituent processes of a method for analyzing smart floor tile impression tile data and/or moulding section sensor data to generate paths, or tracks. In some embodiments, an identity of the person 25.5 may be correlated with the paths or tracks. For example, if the person scanned an ID badge when entering the physical space, their path may be recorded when the person takes their first step on a smart floor tile and their path may be correlated with an identifier received from scanning the badge. In this way, the paths of various people may be recorded (e.g., in a convention hall). This may be beneficial if certain people have desirable job titles (e.g., chief executive officer (CEO), vice president, president, etc.) and/or work at desirable client entities. For example, in some embodiments, the path of a CEO may be tracked during a convention to determine which booths the CEO stopped at and/or an amount of time the CEO spent at each booth. Such data may be used to determine where to place certain booths in the future. For example, if a booth was visited by a threshold number of people having a certain title for a certain period of time, a recommendation may be generated and presented that recommends relocating the booth to a location in the convention hall that is more easily accessible to foot traffic. Likewise, if it is determined that a booth has poor visitation frequency based on the paths, or tracks, of attendees at the convention, a recommendation may be generated to relocate the booth to another location that is more easily accessible to foot traffic. In some embodiments, the machine learning models 154 may be trained to determine the paths, or tracks, of the people having various job titles and working for desired client entities, analyze their paths (e.g., which location the people visited, how long the people visited those locations, etc.), and generate recommendations.

According to certain embodiments, the method comprises the operations of obtaining impression image data, impression tile data, and/or moulding section sensor data from database 430.5, cleaning the obtained image data, impression tile data, and/or moulding section sensor data and reconstructing paths using the cleaned data. In some embodiments, cleaning the data includes removing extraneous sensor data, removing gaps between image data, impression tile data, and/or moulding section sensor data caused by sensor noise, removing long image data, impression tile data, and/or moulding section sensor data caused by objects placed on smart floor tiles, by objects placed in front of moulding sections, by objects stationary in image data, by defective sensors, and sorting image data, impression tile data, and/or moulding section sensor data by start time to produce sorted image data, impression tile data, and/or moulding section sensor data. According to certain embodiments, job workers 425A.5-425C.5 perform processes for reconstructing paths by implementing algorithms that first cluster image data, impression tile data, and/or moulding section sensor data that overlap in time or are spatially adjacent. Next, the clustered data is searched, and pairs of image data, impression tile data, and/or moulding section sensor data that start or end within a few milliseconds of one another are combined into footsteps and/or locations of the object, which are then linked together to form footsteps and/or locations. Footsteps and/or locations are further analyzed and linked to create paths.

According to certain embodiments, database 430.5 provides a repository of raw and processed image data, smart floor tile impression tile data, and/or moulding section sensor data, as well as data relating to the health and status of each of tile controllers 405A.5-405C.5 and moulding sections 102.5. In the non-limiting example shown in FIG. 5000, database 430 is embodied on a server machine communicatively connected to the computing platforms providing API suite 410.5, trigger controller 420.5, and upon which job workers 425A.5-425C.5 execute. According to some embodiments, database 430.5 is embodied on the cloud-based computing system 116.5 as the database 129.5.

In the non-limiting example shown in FIG. 5000, the computing platforms providing trigger controller 420.5 and database 430.5 are communicatively connected to one or more network(s) 20.5. According to embodiments, network 20.5 comprises any network suitable for distributing impression tile data, image data, moulding section sensor data, determined paths, determined gait deterioration of a parameter, determine propensity for a fall event, and control signals (e.g., interventions) based on determined propensities for fall events, including, without limitation, the internet or a local network (for example, an intranet) of a smart building.

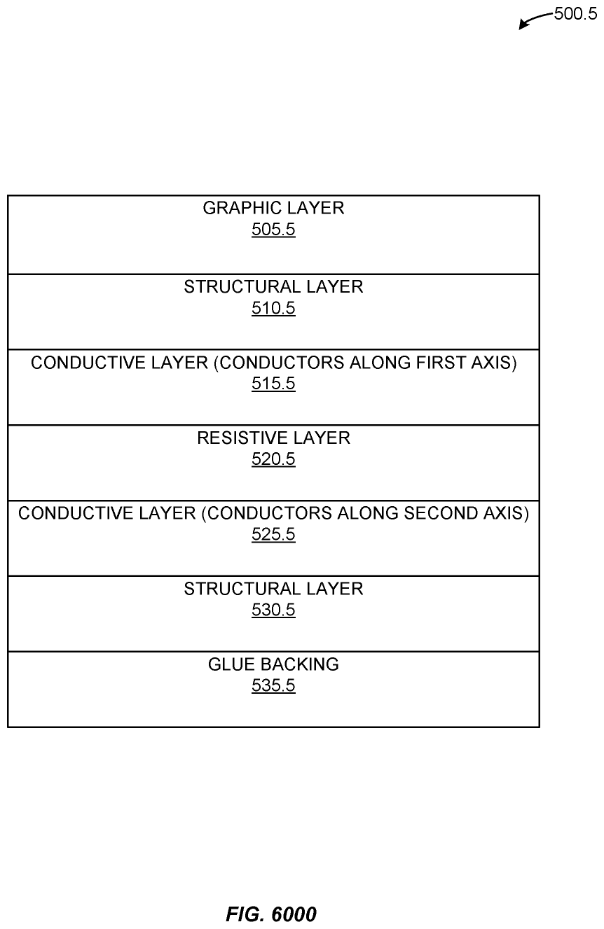

Smart floor tiles utilizing a variety of sensing technologies, such as membrane switches, pressure sensors and capacitive sensors, to identify instances of contact with a floor are within the contemplated scope of this disclosure. FIG. 6000 illustrates aspects of a resistive smart floor tile 500.5 according to certain embodiments of the present disclosure. The embodiment of the resistive smart floor tile 500.5 shown in FIG. 6000 is for illustration only and other embodiments could be used without departing from the scope of the present disclosure.

In the non-limiting example shown in FIG. 6000, a cross section showing the layers of a resistive smart floor tile 500.5 is provided. According to some embodiments, the resistance to the passage of electrical current through the smart floor tile varies in response to contact pressure. From these changes in resistance, values corresponding to the pressure and location of the contact may be determined. In some embodiments, resistive smart floor tile 500.5 may comprise a modified carpet or vinyl floor tile, and have dimensions of approximately 2′×2′.

According to certain embodiments, resistive smart floor tile 500.5 is installed directly on a floor, with graphic layer 505.5 comprising the top-most layer relative to the floor. In some embodiments, graphic layer 505.5 comprises a layer of artwork applied to smart floor tile 500.5 prior to installation. Graphic layer 505.5 can variously be applied by screen printing or as a thermal film.

According to certain embodiments, a first structural layer 510.5 is disposed, or located, below graphic layer 505.5 and comprises one or more layers of durable material capable of flexing at least a few thousandths of an inch in response to footsteps or other sources of contact pressure. In some embodiments, first structural layer 510 may be made of carpet, vinyl or laminate material.

According to some embodiments, first conductive layer 515.5 is disposed, or located, below structural layer 510.5. According to some embodiments, first conductive layer 515.5 includes conductive traces or wires oriented along a first axis of a coordinate system. The conductive traces or wires of first conductive layer 515.5 are, in some embodiments, copper or silver conductive ink wires screen printed onto either first structural layer 510.5 or resistive layer 520.5. In other embodiments, the conductive traces or wires of first conductive layer 515.5 are metal foil tape or conductive thread embedded in structural layer 510.5. In the non-limiting example shown in FIG. 6000, the wires or traces included in first conductive layer 515.5 are capable of being energized at low voltages on the order of 5 volts. In the non-limiting example shown in FIG. 6000, connection points to a first sensor layer of another smart floor tile or to tile controller are provided at the edge of each smart floor tile 500.5.

In various embodiments, a resistive layer 520.5 is disposed, or located, below conductive layer 515.5. Resistive layer 520.5 comprises a thin layer of resistive material whose resistive properties change under pressure. For example, resistive layer 320.5 may be formed using a carbon-impregnated polyethylete film.

In the non-limiting example shown in FIG. 6000, a second conductive layer 525.5 is disposed, or located, below resistive layer 520.5. According to certain embodiments, second conductive layer 525.5 is constructed similarly to first conductive layer 515.5, except that the wires or conductive traces of second conductive layer 525.5 are oriented along a second axis, such that when smart floor tile 500.5 is viewed from above, there are one or more points of intersection between the wires of first conductive layer 515.5 and second conductive layer 525.5. According to some embodiments, pressure applied to smart floor tile 500.5 completes an electrical circuit between a sensor box (for example, tile controller 425.5 as shown in FIG. 5000) and smart floor tile, allowing a pressure-dependent current to flow through resistive layer 520.5 at a point of intersection between the wires of first conductive layer 515.5 and second conductive layer 525.5. The pressure-dependent current may represent a measurement of pressure and the measurement of pressure may be transmitted to the cloud-based computing system 116.5.

In some embodiments, a second structural layer 530.5 resides beneath second conductive layer 525.5. In the non-limiting example shown in FIG. 6000, second structural layer 530.5 comprises a layer of rubber or a similar material to keep smart floor tile 500.5 from sliding during installation and to provide a stable substrate to which an adhesive, such as glue backing layer 535.5 can be applied without interference to the wires of second conductive layer 525.5.

The foregoing description is purely descriptive and variations thereon are contemplated as being within the intended scope of this disclosure. For example, in some embodiments, smart floor tiles according to this disclosure may omit certain layers, such as glue backing layer 535.5 and graphic layer 505.5 described in the non-limiting example shown in FIG. 6000.

According to some embodiments, a glue backing layer 535.5 comprises the bottom-most layer of smart floor tile 500.5. In the non-limiting example shown in FIG. 6000, glue backing layer 535.5 comprises a film of a floor tile glue.

FIG. 7000 illustrates a master control device 600.5 according to certain embodiments of this disclosure. FIG. 7000 illustrates a master control device 600.5 according to certain embodiments of this disclosure. The embodiment of the master control device 600.5 shown in FIG. 7000 is for illustration only and other embodiments could be used without departing from the scope of the present disclosure.

In the non-limiting example shown in FIG. 7000, master control device 600.5 is embodied on a standalone computing platform connected, via a network, to a series of end devices (e.g., tile controller 405A.5 in FIG. 5000) in other embodiments, master control device 600.5 connects directly to, and receives raw signals from, one or more smart floor tiles (for example, smart floor tile 500.5 in FIG. 6000). In some embodiments, the master control device 600.5 is implemented on a server 128.5 of the cloud-based computing system 116.5 in FIG. 2000B and communicates with the smart floor tiles 112.5, the moulding sections 102.5, the camera 50.5, the computing device 12.5, the computing device 15.5, and/or the electronic device 13.5.

According to certain embodiments, master control device 600.5 includes one or more input/output interfaces (I/O) 605.5. In the non-limiting example shown in FIG. 7000, I/O interface 605.5 provides terminals that connect to each of the various conductive traces of the smart floor tiles deployed in a physical space. Further, in systems where membrane switches or smart floor tiles are used as mat presence sensors, I/O interface 605 electrifies certain traces (for example, the traces contained in a first conductive layer, such as conductive layer 515.5 in FIG. 6000) and provides a ground or reference value for certain other traces (for example, the traces contained in a second conductive layer, such as conductive layer 525.5 in FIG. 6000). Additionally, I/O interface 605.5 also measures current flows or voltage drops associated with occupant presence events, such as a person's foot squashing a membrane switch to complete a circuit, or compressing a resistive smart floor tile, causing a change in a current flow across certain traces. In some embodiments, I/O interface 605.5 amplifies or performs an analog cleanup (such as high or low pass filtering) of the raw signals from the smart floor tiles in the physical space in preparation for further processing.

In some embodiments, master control device 600.5 includes an analog-to-digital converter (“ADC”) 610.5. In embodiments where the smart floor tiles in the physical space output an analog signal (such as in the case of resistive smart floor tile), ADC 610.5 digitizes the analog signals. Further, in some embodiments, ADC 610.5 augments the converted signal with metadata identifying, for example, the trace(s) from which the converted signal was received, and time data associated with the signal. In this way, the various signals from smart floor tiles can be associated with touch events occurring in a coordinate system for the physical space at defined times. While in the non-limiting example shown in FIG. 7000, ADC 610.5 is shown as a separate component of master control device 600.5, the present disclosure is not so limiting, and embodiments wherein ADC 610.5 is part of, for example, I/O interface 605.5 or processor 615.5 are contemplated as being within the scope of this disclosure.

In various embodiments, master control device 600.5 further comprises a processor 615.5. In the non-limiting example shown in FIG. 7000, processor 615.5 is a low-energy microcontroller, such as the ATMEGA328P by Atmel Corporation. According to other embodiments, processor 615.5 is the processor provided in other processing platforms, such as the processors provided by tablets, notebook or server computers.

In the non-limiting example shown in FIG. 7000, master control device 600.5 includes a memory 620.5. According to certain embodiments, memory 620.5 is a non-transitory memory containing program code to implement, for example, APIs 625.5, networking functionality and the algorithms for generating and analyzing paths described herein.

Additionally, according to certain embodiments, master control device 600.5 includes one or more Application Programming Interfaces (APIs) 625.5. In the non-limiting example shown in FIG. 7000, APIs 625.5 include APIs for determining and assigning break points in one or more streams of smart floor tile data and/or moulding section sensor data and defining data sets for further processing. Additionally, in the non-limiting example shown in FIG. 7000, APIs 625.5 include APIs for interfacing with a job scheduler (for example, trigger controller 420.5 in FIG. 4) for assigning batches of data to processes for analysis and determination of paths. According to some embodiments, APIs 625.5 include APIs for interfacing with one or more reporting or control applications provided on a client device. Still further, in some embodiments, APIs 625.5 include APIs for storing and retrieving image data, smart floor tile data, and/or moulding section sensor data in one or more remote data stores (for example, database 430.5 in FIG. 5000, database 129.5 in FIG. 2000B, etc.).

According to some embodiments, master control device 600.5 includes send and receive circuitry 630.5, which supports communication between master control device 600.5 and other devices in a network context in which smart building control using directional occupancy sensing is being implemented according to embodiments of this disclosure. In the non-limiting example shown in FIG. 7000, send and receive circuitry 630.5 includes circuitry 635.5 for sending and receiving data using Wi-Fi, including, without limitation at 900 MHz, 2.8 GHz and 5.0 GHz. Additionally, send and receive circuitry 630.5 includes circuitry, such as Ethernet circuitry 640.5 for sending and receiving data (for example, smart floor tile data) over a wired connection. In some embodiments, send and receive circuitry 630.5 further comprises circuitry for sending and receiving data using other wired or wireless communication protocols, such as Bluetooth Low Energy or Zigbee circuitry.

Additionally, according to certain embodiments, send and receive circuitry 630.5 includes a network interface 650.5, which operates to interconnect master control device 600.5 with one or more networks. Network interface 650.5 may, depending on embodiments, have a network address expressed as a node ID, a port number or an IP address. According to certain embodiments, network interface 650.5 is implemented as hardware, such as by a network interface card (NIC). Alternatively, network interface 650.5 may be implemented as software, such as by an instance of the java.net.NetworkInterface class. Additionally, according to some embodiments, network interface 650.5 supports communications over multiple protocols, such as TCP/IP as well as wireless protocols, such as 3G or Bluetooth.

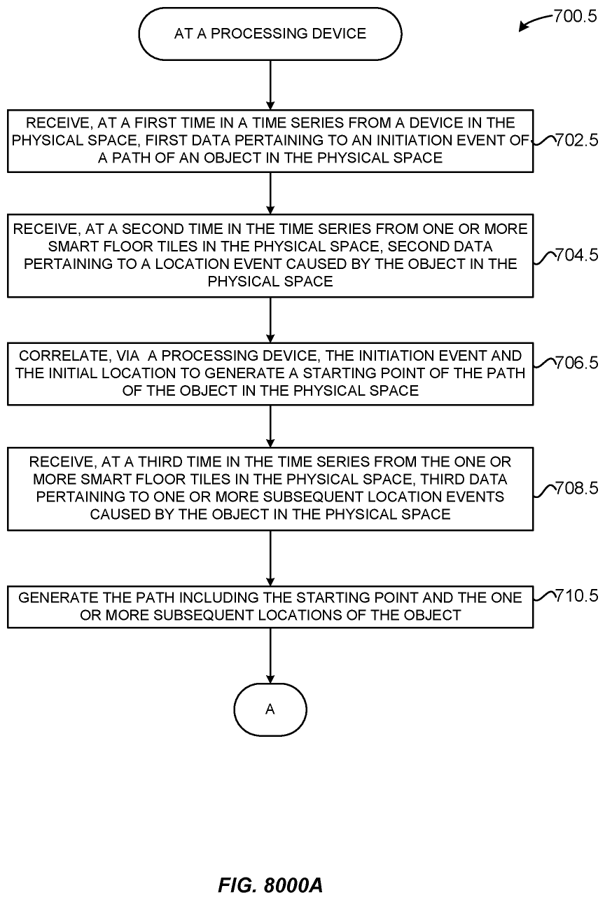

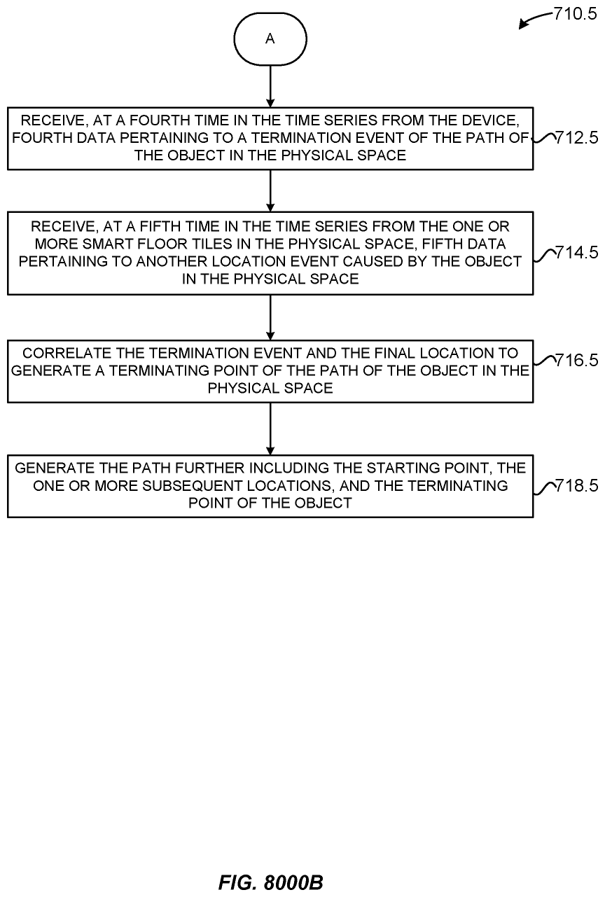

FIG. 8000A illustrate an example of a method 700.5 for generating a path of a person in a physical space using smart floor tiles 112.5 according to certain embodiments of this disclosure. The method 700.5 may be performed by processing logic that may include hardware (circuitry, dedicated logic, etc.), software, or a combination of both. The method 700.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000B) implementing the method 700.5. The method 700.5 may be implemented as computer instructions stored on a memory device and executable by the one or more processors. In certain implementations, the method 700.5 may be performed by a single processing thread. Alternatively, the method 700.5 may be performed by two or more processing threads, each thread implementing one or more individual functions, routines, subroutines, or operations of the methods.

At block 702.5, the processing device may receive, at a first time in a time series, from a device (e.g., camera 50.5, reader device, etc.) in a physical space (first room 21.5), first data pertaining to an initiation event of the path of the object (e.g., person 25.5) in the physical space. The first data may include an identity of the person, employment position of the person in an entity, a job title of the person, an entity identity that employs the person, a gender of the person, an age of the person, a timestamp of the data, and the like. The initiation event may correspond to the person checking in for an event being held in the physical space. In some embodiments, when the device is a camera 50.5, the processing device may perform facial recognition techniques using facial image data received from the camera 50.5 to determine an identity of the person. The processing device may obtain information pertaining to the person based on the identity of the person. The information may include an entity for which the person works, an employment position of the person within the entity, or some combination thereof.

At block 704.5, the processing device may receive, at a second time in the time series from one or more smart floor tiles 112.5 in the physical space, second data pertaining to a location event caused by the object in the physical space. The location event may include an initial location of the object in the physical space. The initial location may be generated by one or more detected forces at the one or more smart floor tiles 112.5. The second data may be impression tile data received when the person steps onto a first smart floor tile 112.5 in the physical space. In some embodiments, the person may be standing on the first smart floor tile 112.5 when the initiation event occurs. That is, the initiation event and the location event may occur contemporaneously at substantially the same time in the time series. In some embodiments, the first time and the second time may differ less than a threshold period of time, or the first time and the second time may be substantially the same. The location event may include data pertaining to the one or more smart tiles 112.5 the object pressed, such as an identifier of the one or more smart floor tiles 112.5, a timestamp of when the one or more smart floor tiles 112.5 changed from an idle state to an active state, a duration of being in the active state, and the like.

At block 706.5, the processing device may correlate the initiation event and the initial location to generate a starting point of a path of the object in the physical space. In some embodiments, the starting point may be overlaid on a virtual representation of the physical space and the path of the object may be generated and presented in real-time or near real-time as the object moves around the physical space.

At block 708.5, the processing device may receive, at a third time in the time series from the one or more smart floor tiles 112.5 in the physical space, third data pertaining to one or more subsequent location events caused by the object in the physical space. The one or more subsequent location events may include one or more subsequent locations of the object in the physical space. The one or more subsequent location events may include data pertaining to the one or more smart tiles 112.5 the object pressed, such as an identifier of the one or more smart floor tiles 112.5, a timestamp of when the one or more smart floor tiles 112.5 changed from an idle state to an active state, a duration of being in the active state, and the like.

At block 709.5, the processing device may generate the path of the object including the starting point and the one or more subsequent locations of the object.

FIG. 8000B illustrates an example of a method 710.5 continued from FIG. 8000A according to certain embodiments of this disclosure. The method 710.5 may be performed by processing logic that may include hardware (circuitry, dedicated logic, etc.), software, or a combination of both. The method 710.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 8000B) implementing the method 710.5. The method 710.5 may be implemented as computer instructions stored on a memory device and executable by the one or more processors. In certain implementations, the method 710.5 may be performed by a single processing thread. Alternatively, the method 710.5 may be performed by two or more processing threads, each thread implementing one or more individual functions, routines, subroutines, or operations of the methods.

At block 712.5, the processing device may receive, at a fourth time in the time series from a device (e.g., camera 50.5, reader, etc.), fourth data pertaining to a termination event of the path of the object in the physical space.

At block 714.5, the processing device may receive, at a fifth time in the time series from the one or more smart floor tiles 112.5 in the physical space, fifth data pertaining to another location event caused by the object in the physical space. The another location event may correspond to when the user leaves the physical space (e.g., by checking out with a badge or any electronic device). The another location event may include a final location of the object in the physical space. The another location event may include data pertaining to the one or more smart tiles 112.5 the object pressed, such as an identifier of the one or more smart floor tiles 112.5, a timestamp of when the one or more smart floor tiles 112.5 changed from an idle state to an active state, a duration of being in the active state, and the like.

At block 716.5, the processing device may correlate the termination event and the final location to generate a terminating point of the path of the object in the physical space.

At block 718.5, the processing device may generate the path using the starting point, the one or more subsequent locations, and the terminating point of the object. Block 718.5 may result in the full path of the object in the physical space. The full path may be presented on a user interface of a computing device.

In some embodiments, the processing device may generate a second path for a second person in the physical space. The processing device may generate an overlay image by overlaying the path of the first person with the second path of the second object in a virtual representation of the physical space. The different paths may be represented using different or the same visual elements (e.g., color, boldness, etc.). The processing device may cause the overlay image to be presented on a computing device.

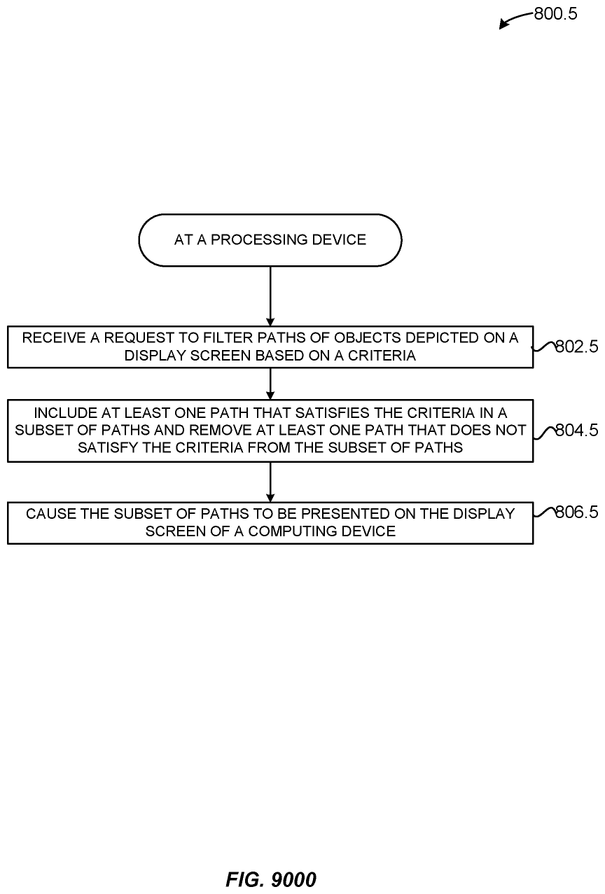

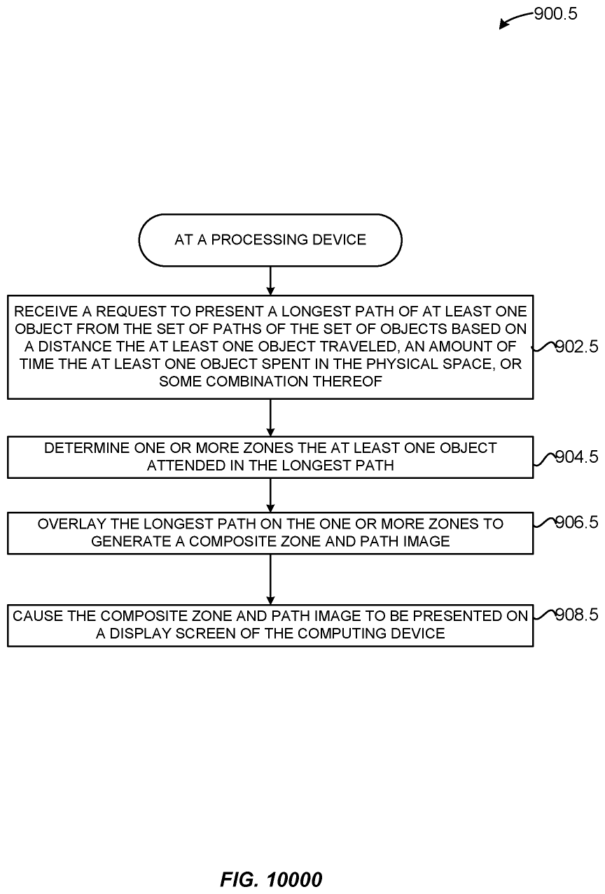

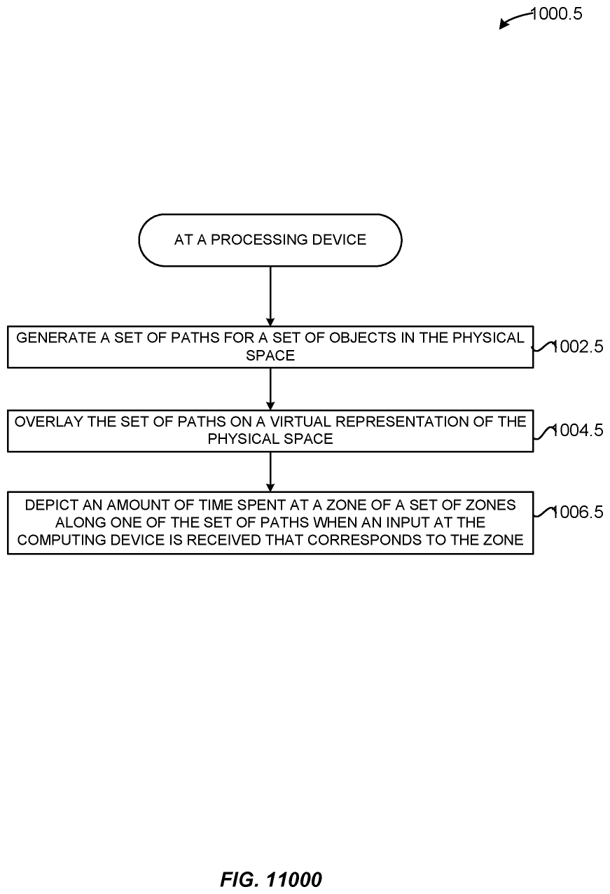

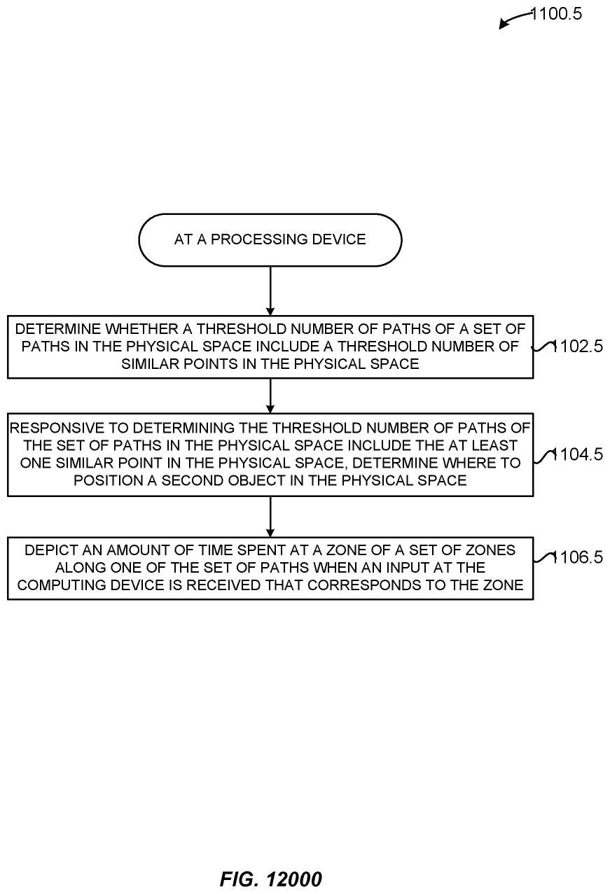

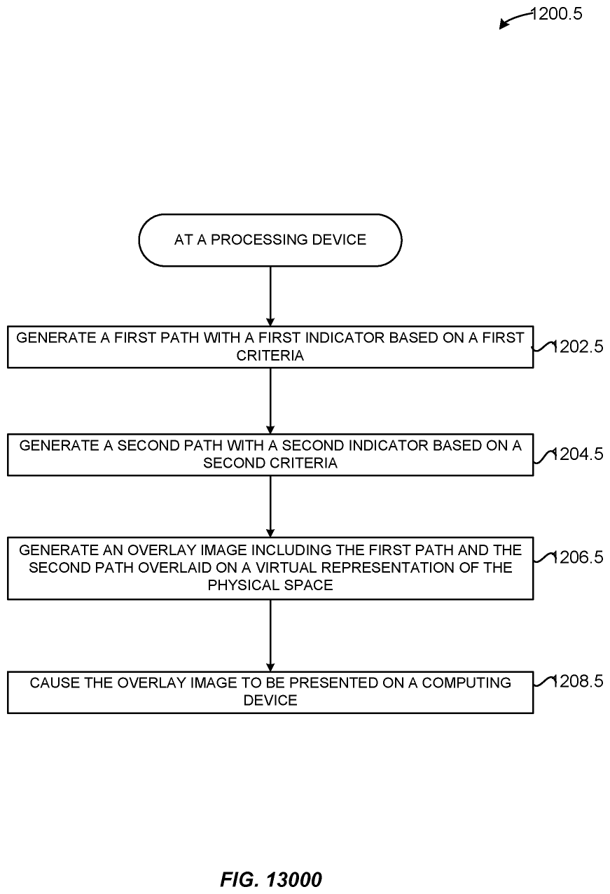

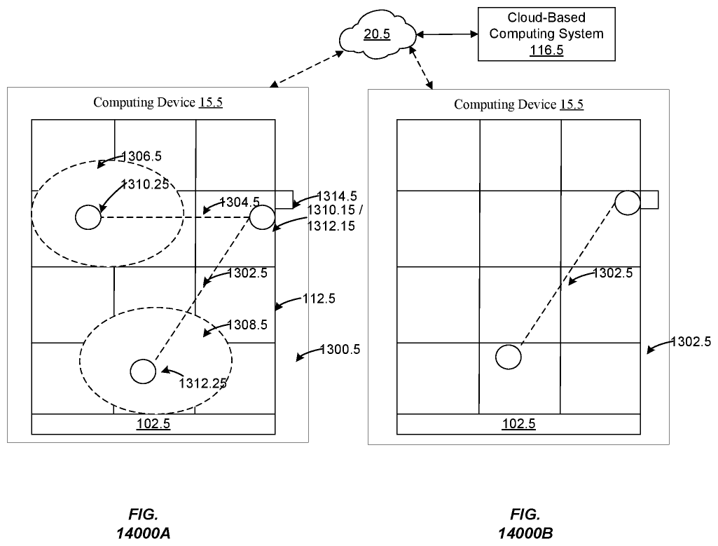

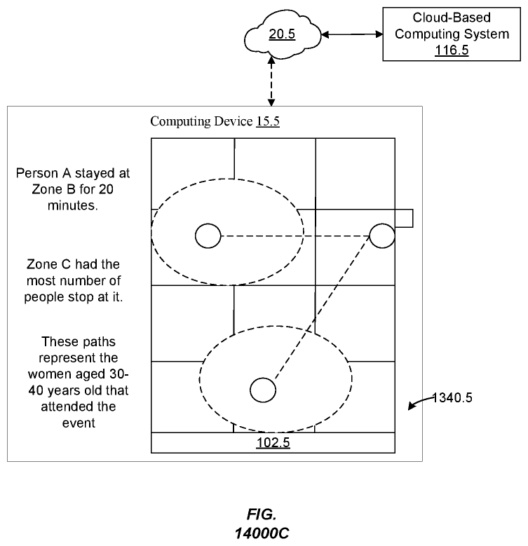

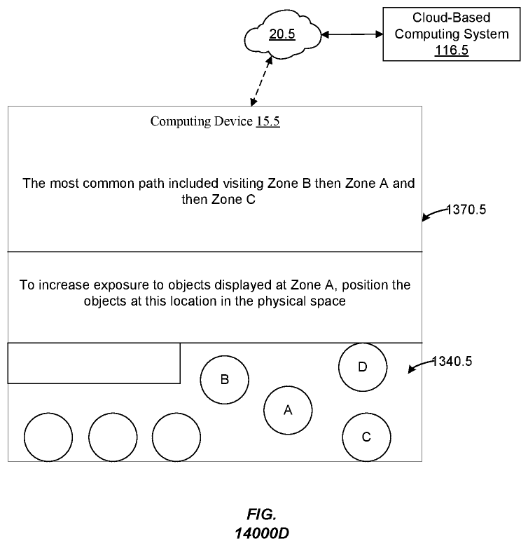

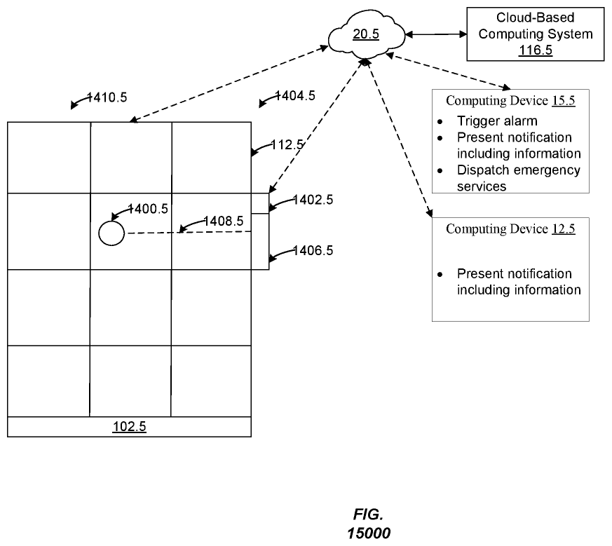

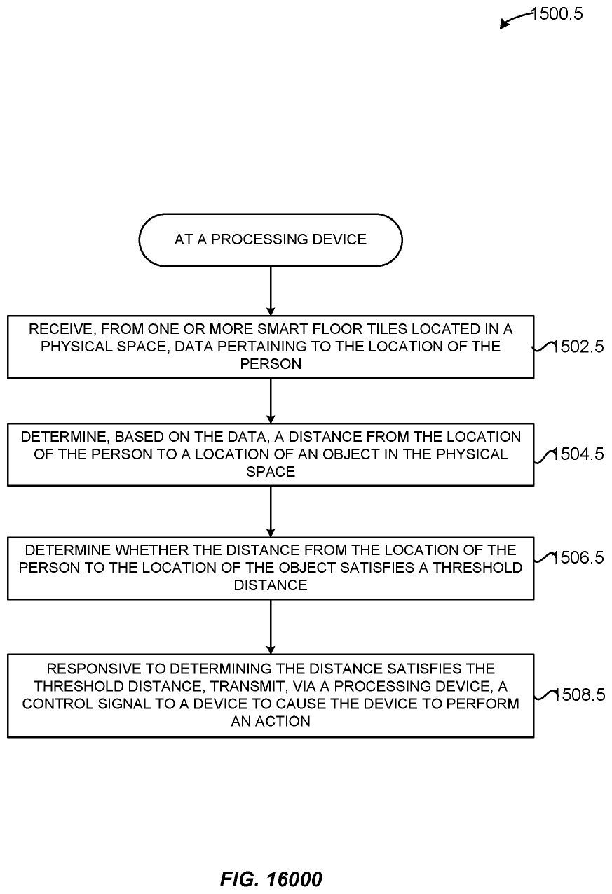

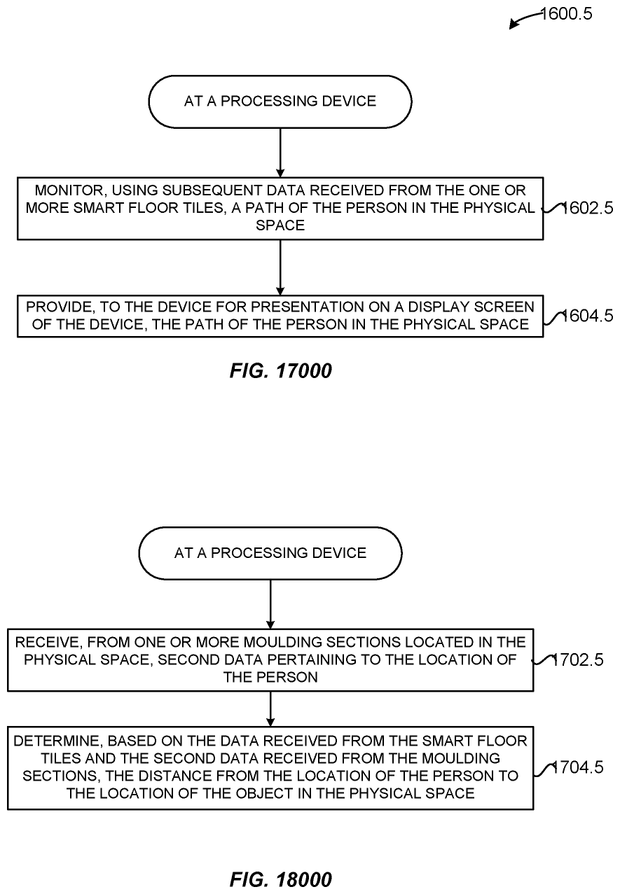

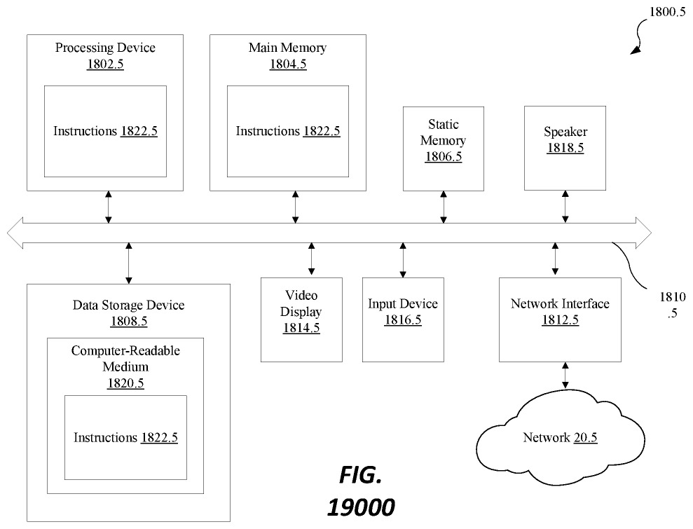

FIG. 9000 illustrates an example of a method 800.5 for filtering paths of objects presented on a display screen according to certain embodiments of this disclosure. The method 800.5 may be performed by processing logic that may include hardware (circuitry, dedicated logic, etc.), software, or a combination of both. The method 800.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000B) implementing the method 800.5. The method 800.5 may be implemented as computer instructions stored on a memory device and executable by the one or more processors. In certain implementations, the method 800.5 may be performed by a single processing thread. Alternatively, the method 800.5 may be performed by two or more processing threads, each thread implementing one or more individual functions, routines, subroutines, or operations of the methods.

At block 802.5, the processing device may receive a request to filter paths of objects depicted on a user interface of a display screen based on a criteria. The criteria may be employment position, job title, entity identity for which people work, gender, age, or some combination thereof.

At block 804.5, the processing device may include at least one path that satisfies the criteria in a subset of paths and remove at least one path that does not satisfy the criteria from the subset of paths. For example, if the user selects to view paths of people having a manager position, the processing device may include the paths of all manager positions and remove other paths of people that do not have the manager position.

At block 806.5, the processing device may cause the subset of paths to be presented on the display screen of a computing device. The subset of paths may provide an improved user interface that increases the user's experience using the computing device because it includes only the desired paths of people in the physical area. Further, computing resources may be reduced by generating the subset of paths because fewer paths may be generated based on the criteria. Also less data may be transmitted over the network to the computing device displaying the subset because there are fewer paths in the subset based on the criteria.

FIG. 10000 illustrates an example of a method 900.5 for presenting a longest path of an object in a physical space according to certain embodiments of this disclosure. The method 900.5 may be performed by processing logic that may include hardware (circuitry, dedicated logic, etc.), software, or a combination of both. The method 900.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000A) implementing the method 900.5. The method 900.5 may be implemented as computer instructions stored on a memory device and executable by the one or more processors. In certain implementations, the method 900.5 may be performed by a single processing thread. Alternatively, the method 900.5 may be performed by two or more processing threads, each thread implementing one or more individual functions, routines, subroutines, or operations of the methods.

At block 902.5, the processing device may receive a request to present a longest path of at least one object from the set of paths of the set of objects (e.g., people) based on a distance at least one object traveled, an amount of time the at least one object spent in the physical space, or some combination thereof.

At block 904.5, the processing device may determine one or more zones the at least one object attended in the longest path. The one or more zones may be determined using a virtual representation of the physical space and selecting the zones including smart floor tiles 112.5 through which the path of the at least one object traversed.

At block 906.5, the processing device may overlay the longest path of the at least one object on the one or more zones to generate a composite zone and path image.

At block 908.5, the processing device may cause the composite zone and path image to be presented on a display screen of the computing device. In some embodiments, the shortest path may also be selected and presented on the display screen. The longest path and the shortest path may be presented concurrently. In some embodiments, any suitable length of path in any combination may be selected and presented on a virtual representation of the physical space as desired.

FIG. 11000 illustrates an example of a method 1000.5 for presenting amount of times objects spent at certain zones in a physical space according to certain embodiments of this disclosure. The method 1000.5 may be performed by processing logic that may include hardware (circuitry, dedicated logic, etc.), software, or a combination of both. The method 1000.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000A) implementing the method 1000.5. The method 1000.5 may be implemented as computer instructions stored on a memory device and executable by the one or more processors. In certain implementations, the method 1000.5 may be performed by a single processing thread. Alternatively, the method 1000.5 may be performed by two or more processing threads, each thread implementing one or more individual functions, routines, subroutines, or operations of the methods.

At block 1002.5, the processing device may generate a set of paths for a set of objects in the physical space. At block 1004, the processing device may overlay the set of paths on a virtual representation of the physical space.

At block 1006.5, the processing device may depict an amount of time spent at a zone of a set of zones along one of the set of paths when an input at the computing device is received that corresponds to the zone. In some embodiments, the user may select any point on the path of any person to determine the amount of time that person spent at a location at the selected point. Granular location and duration details may be provided using the data obtained via the smart floor tiles 112.5.

FIG. 12000 illustrates an example of a method 1100.5 for determining where to place objects based on paths of people according to certain embodiments of this disclosure. The method 1100.5 may be performed by processing logic that may include hardware (circuitry, dedicated logic, etc.), software, or a combination of both. The method 1100.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000A) implementing the method 1100.5. The method 1100.5 may be implemented as computer instructions stored on a memory device and executable by the one or more processors. In certain implementations, the method 1100.5 may be performed by a single processing thread. Alternatively, the method 1100.5 may be performed by two or more processing threads, each thread implementing one or more individual functions, routines, subroutines, or operations of the methods.

At block 1102.5, the processing device may determine whether a threshold number of paths of a set of paths in the physical space include a threshold number of similar points in the physical space. At block 1104.5, responsive to determining the threshold number of paths of the set of paths in the physical space include the at least one similar point in the physical space, the processing device may determine where to position a second object in the physical space. At block 1106.5, the processing device may depict an amount of time spent at a zone of a set of zones along one of the set of paths when an input at the computing device is received that corresponds to the zone, a person, a path, a booth, or the like.

FIG. 13000 illustrates an example of a method 1200.5 for overlaying paths of objects based on criteria according to certain embodiments of this disclosure. The method 1200.5 may be performed by processing logic that may include hardware (circuitry, dedicated logic, etc.), software, or a combination of both. The method 1200.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000A) implementing the method 1200.5. The method 1200.5 may be implemented as computer instructions stored on a memory device and executable by the one or more processors. In certain implementations, the method 1200.5 may be performed by a single processing thread. Alternatively, the method 1200.5 may be performed by two or more processing threads, each thread implementing one or more individual functions, routines, subroutines, or operations of the methods.

At block 1202.5, the processing device may generate a first path with a first indicator based on a first criteria. The criteria may be job title, company name, age, gender, longest path, shortest path, etc. The first indicator may be a first color for the first path.

At block 1204.5, the processing device may generate a second path with a second indicator based on a second criteria. At block 1206.5, the processing device may generate an overlay image including the first path and the second path overlaid on a virtual representation of the physical space. At block 1208.5, the processing device may cause the overlay image to be presented on a computing device.

FIG. 14000A illustrates an example user interface 1300.5 presenting paths 1300.5 and 1304.5 of people in a physical space according to certain embodiments of this disclosure. More particularly, the user interface 1300.5 presents a virtual representation of the first room 21.5, for example, from an above perspective. The user interface 1300.5 presents the smart floor tiles 112.5 and/or moulding section 102.5 that are arranged in the physical space. The user interface 1300.5 may include a visual representation mapping various zones 1306.5 and 1308.5 including various booths in the physical space.

An entrance to the physical space may include a device 1314.5 at which the user checks in for the event being held in the physical space. The device 1314.5 may be a reader device and/or a camera 50.5. The device 1314.5 may send data to the cloud-based computing system 116.5 to perform the methods disclosed herein.

For example, the data may be included in an initiation event that is used to generate a starting point of the path of the person. When the person enters the physical space, the person may press one or more first smart floor tiles 112.5 that transmit measurement data to the cloud-based computing system 116.5. The measurement data may be included in a location event and may include an initial location of the person in the physical space. The initial location and the initiation event may be used to generate the starting position of the path of the person. The measurement data obtained by the smart floor tiles 112.5 and sent to the cloud-based computing system 116.5 may be used during later location events and a termination location event to generate a full path of the person.

As depicted, two starting points 1310.15 and 1312.15 are overlaid on a smart floor tile 112.5 in the user interface 1300.5. Starting point 1310.15 is included as part of path 1304.5 and starting point 1312.15 is included as part of path 1302.5. Termination points 1310.25 and 1312.25. The termination point 1310.25 ends in zone 1306.5 and termination point 1312.25 ends in zone 1308.5. If the user places the cursor or selects any portion of the path (e.g., using a touchscreen), additional details of the paths 1304.5 and 1302.5 may be presented. For example, a duration of time the person spent at any of the points in the paths 1304.5 may be presented.

FIG. 14000B illustrates an example user interface 1302.5 presenting a filtered path of a person in a physical space according to certain embodiments of this disclosure. In some embodiments, the paths presented in the user interface 1302.5 may be filtered based on any suitable criteria. For example, the user may select to view the paths of a person having a certain employment positon (e.g., a chief level position), and the user interface 1300.5 presents the path 1302.5 of the person having the certain employment position and removes the path 1304.5 of the person that does not have that employment position.

FIG. 14000C illustrates an example user interface 1304.5 presenting information pertaining to paths of people in a physical space according to certain embodiments of this disclosure. As depicted, the user interface 1340.5 presents “Person A stayed at Zone B for 20 minutes”, “Zone C had the most number of people stop at it”, and “These paths represent the women aged 30-40 years old that attended the event.” As may be appreciated, the improve user interface 1304.5 may greatly enhance the experience of a user using the computing device 15.5 as the analytics enabled and disclosed herein may be very beneficial. Any suitable subset of paths may be generated using any suitable criteria.

FIG. 14000D illustrates an example user interface 1370.5 presenting other information pertaining to a path of a person in a physical space and a recommendation where to place an object in the physical space based on path analytics according to certain embodiments of this disclosure. As depicted, the user interface 1370.5 presents “The most common path included visiting Zone B then Zone A and then Zone C”. The cloud-based computing system 116.5 may analyze the paths by comparing them to determine the most common path, the least common path, the durations spent at each zone, booth, or object in the physical space, and the like.

The user interface 1370.5 also presents “To increase exposure to objects displayed at Zone A, position the objects at this location in the physical space”. A visual representation 1372.5 presents the recommended location for objects in Zone A relative to other Zones B, C, and D. Accordingly, the cloud-based computing system 116.5 may determine the ideal locations for increasing traffic and/or attendance in zones and may recommend where to locate the zones, the booths in the zones, and/or the objects displayed at particular booths based on path analytics performed herein.

FIG. 15000 illustrates an example for performing, based on a location of a person 1400.5, one or more actions using one or more devices 1402.5 according to certain embodiments of this disclosure. As depicted, the person may be present in a physical space 1404.5 that includes installed smart floor tiles 112.5 and moulding sections 102.5. The physical space 1404.5 also includes an object 1406.5 (e.g., a door, window, or the like) that is located at an ingress and egress of the physical space 1404.5. The object also includes an installed device 1402.5.

As discussed herein, a location of the person 1400.5 may be determined by the cloud-based computing system 116.5 based on data received from the smart floor tiles 112.5 via the network 20.5. The cloud-based computing system 116.5 may determine a distance 1408.5 of the location of the person 1400.5 from a location of the object 1406.5. In some embodiments, the physical space may be represented as a virtual representation of the physical space 1404.5. For example, The virtual representation 1410.5 may depict a layout of the smart floor tiles 112.5 in a grid and may depict the object 1406.5, device 1402.5, and/or moulding sections. Further, the virtual representation 1410.5 may overlay a representation of the person 1400.5 on the virtual representation 1410.5. When the distance 1406.5 between the location of the person 1400.5 and the location of the object 1406.5 satisfies a threshold distance, then the cloud-based computing system 116.5 may transmit a control signal to the device 1402.5 to cause the device 1402.5 to perform an action. In some embodiments, the cloud-based computing system 116.5 may transmit the control signal to the computing device 15.5 and/or the computing device 12.5.

The device 1402.5 may be an actuation mechanism. In some embodiments, the device 1402.5 may be separate from the object 1406.5. For example, the device 1402.5 may be the electronic device 13.5.

In embodiments where the device is an actuation mechanism, the actuation mechanism may be an electronic lock. The electronic lock may include a processing device, a memory device, a network interface device, and/or a locking mechanism that are communicatively coupled. The cloud-based computing system 116.5 may be communicatively coupled to the electronic lock and may be capable of transmitting control signals to the network interface device of the electronic lock. The electronic lock may include any component described with reference to FIG. 19000. The network interface device of the electronic lock may transmit the control signal to the processing device, which may receive the control signal and execute one or more instructions stored in the memory device of the electronic lock to cause the locking mechanism to actuate. The locking mechanism may actuate by locking or unlocking.

In embodiments where the device is an actuation mechanism, the actuation mechanism may be an electromechanical arm. The electromechanical arm may include a processing device, a memory device, a network interface device, and/or a actuating arm mechanism that are communicatively coupled. The cloud-based computing system 116.5 may be communicatively coupled to the electromechanical arm and may be capable of transmitting control signals to the network interface device of the electromechanical arm. The electromechanical arm may include any component described with reference to FIG. 19000. The network interface device of the electromechanical arm may transmit the control signal to the processing device, which may receive the control signal and execute one or more instructions stored in the memory device of the electromechanical arm to cause the actuating arm mechanism to actuate. The actuating arm mechanism may actuate by extending or retracting. The extension of the actuating arm mechanism may cause the object to close (e.g., push a door or window shut), and/or the retraction of the actuating arm mechanism may cause the object to open (e.g., pull a door or window open), or vice versa. The actuating arm mechanism may include one or more hydraulic, pneumatic, spring-based, etc. components capable of causing the actuating arm mechanism to extend and/or retract in response to a control signal from sent from the processing device.

In some embodiments, where the device is the electronic device 13.5, a control signal received from the cloud-based computing system 116.5 may cause the electronic device to trigger an alarm, emit audio via a speaker, emit a light (e.g., strobe light, continuous light to a pathway, etc.), or the like.

In some embodiments, when the cloud-based computing system 116.5 transmits a control signal to the computing device 15.5 of the third-party (e.g., medical personnel, emergency responder, etc.), the computing device 15 may receive the control signal and perform an action. The action may include triggering an alarm on the computing device 15.5, the computing device 12.5, and/or the electronic device 13.5. In some embodiments, the action may include presenting a notification including information. The information may include details about the person 1400.5 (e.g., name, age, gender, medical conditions, etc.), the location of the person 1400.5, an instruction to track down the person 1400.5 and/or escort the person 1400.5 to another location, or the like. In some embodiments, the action may include dispatching emergency services.

In some embodiments, when the cloud-based computing system 116.5 transmits a control signal to the computing device 12.5 of the person 1400.5 (e.g., patient), the computing device 12.5 may receive the control signal and perform an action. The action may include presenting a notification including information. The information may include an instruction for the person 1400.5 to return to another location and/or to move away from the object 1406.5. In some embodiments, the instruction may instruct the person 1400.5 to exit through the object 1406.5 (e.g., in an emergency scenario, such as a fire in the physical space 1404.5). In some embodiments, in the instruction may instruct the user to contact emergency services (e.g., in an emergency scenario, such as a hostile person present in the physical space 1404.5).

FIG. 16000 illustrates an example of a method 1500.5 for performing an action based on a location of a person according to certain embodiments of this disclosure. The method 1500.5 may be performed by processing logic that may include hardware (circuitry, dedicated logic, etc.), software, or a combination of both. The method 1500.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000A) implementing the method 1500.5. The method 1500.5 may be implemented as computer instructions stored on a memory device and executable by the one or more processors. In certain implementations, the method 1500.5 may be performed by a single processing thread. Alternatively, the method 1500.5 may be performed by two or more processing threads, each thread implementing one or more individual functions, routines, subroutines, or operations of the methods.

At block 1502.5, the processing device may receive, from one or more smart floor tiles 112.5 located in a physical space, data pertaining to the location of a person. In some embodiments, the physical space is a nursing home, a hospital, a school, a movie theater, a theater, a stadium, an office, a house, an airport, a bus station, a train station, a port, an auditorium, a cafeteria, a restaurant, a building, a park, a parking garage, or some combination thereof.

In some embodiments, the data may pertain to a location of an animal (e.g., dog, cat, etc.), a robot, any suitable object capable of movement, etc. The one or more smart floor tiles 112.5 may include one or more sensing devices capable of obtaining one or more pressure measurements, and the data received by the processing device may include the one or more pressure measurements. The one or more pressure measurements may include an identity of the one or more smart floor tiles that detected the pressure, a value of the pressure, a timestamp of the pressure, and so forth. The one or more pressure measurements received from the smart floor tiles 112.5 may enable the processing device to accurately determine the location of the person in the physical space because the pressure measurements may be used to identify exactly which smart floor tiles the person is stepping on. A map, grid, data structure, table, or the like may be used to maintain a layout of the various smart floor tiles 112.5 located in the physical space. For example, the map, grid, data structure, table, or the like may store a unique identifier for each of the smart floor tiles 112.5 in the physical space, an identifier of the physical space, and so forth, such that when pressure measurements are received from the smart floor tiles, the processing device determines exactly which smart floor tiles in the physical space are being stepped on.

At block 1504.5, the processing device may determine, based on the data, a distance from the location of the person to a location of an object in the physical space. The processing device may maintain a representation of the physical space including dimensions of the physical space. In some embodiments, the processing device may maintain a virtual representation of the physical space that includes coordinates and objects placed on the coordinates to match their physical location in the physical space. The processing device may determine the distance from the location of the person to the location of a certain object by overlaying the person at the determined location the virtual representation of the physical space. Then, the processing device may measure, using the virtual representation, a distance between the location of the person and the object. The virtual representation may not be represented of the actual size of the physical space so a conversion function may be used to account for actual distance (e.g., the virtual representation may be represented at a lower scale than the actual size of the physical space). The conversion function may account for the size disparity by increasing the determined distance proportionally.

In some embodiments, the processing device may determine the distance between the location of the person and the location of the object by determining first coordinates associated with the location of the person using the virtual representation and second coordinates associated with the location of the object using the virtual representation. The processing device may use the first and second coordinates to determine the distance between the person and the object.

In some embodiments, prior to determining the distance from the location of the person to the location of the object, the processing device may determine an identity of the person based on second data. The second data may include an identifier of the physical space in which the one or more smart floor tiles are located (e.g., each room in a nursing home may be assigned an identifier and the identifier of the physical space may be correlated with an identifier of a patient assigned to that physical space; thus, knowing the identifier of the physical space may enable determining the identity of the patient), an identifier of the person associated with the identifier of the physical space (e.g., the identifier may be read from a scanning device (e.g., RFID), may be determined by searching a data store with the identifier of the physical space, etc.), a weight of the person determined based on the one or more pressure measurements, a time of day the data is received (e.g., there may be a schedule of events for patients in the physical space), an image of the person obtained via the camera in the physical space (e.g., the cloud-based computing system 116.5 may determine the identity using facial recognition on the image), a stored image of the person (e.g., the stored image may be compared to the image obtained by the camera to identify the person), or some combination thereof. The processing device may determine whether the identity of the person is included in a list. The list may be any suitable list of people of interest. The people of interest may be people having certain medical conditions, patients, people having certain criminal records, or any suitable criteria for being placed on a list of people to watch and/or monitor.

At block 1506.5, the processing device may determine whether the distance from the location of the person to the location of the object satisfies a threshold distance. The threshold distance may be configurable and may be any suitable distance (e.g., 1 foot, 5 feet, 10 feet, etc.). As previously discussed, in certain scenarios, it may be desirable to perform an action when a person is within a threshold distance from certain objects. For example, in a nursing home, it may be desirable to cause a door to lock when a person with a neurodegenerative disease walks within a threshold distance of the door to prevent the person from wandering outside of the physical space and potentially getting lost and/or hurt.

At block 1508.5, responsive to determine the distance satisfies the threshold distance, the processing device may transmit a control signal to a device to cause the device to perform an action. The device may be distal from the processing device. For example, the processing device may be operating in the cloud-based computing system 116.5 and the device may be physically present in the physical space distally from the processing device.

In some embodiments, the object is an ingress and/or egress to the physical space. In some embodiments, the object includes a door or a window, the device includes an actuation mechanism, and the action includes causing the actuation mechanism to actuate. For example, the actuation mechanism may be a lock and/or an electromechanical arm. Causing the actuation mechanism to actuate may include (i) actuating the actuation mechanism to lock or unlock, (ii) actuating the actuation mechanism to open or close the door or the window, or (iii) some combination thereof.

In some embodiments, the action performed by the device may include (i) actuating to open the object, to close the object, to lock the object, to unlock the object, or some combination thereof, (ii) presenting, on a display screen of the device, a notification including information pertaining to the person, the location of the person, the distance satisfying the threshold distance, or some combination thereof, (iii) triggering an alarm, (iv) enabling, via a speaker of the device, communicating with the person in the physical space, (v) dispatching an emergency service, or (v) some combination thereof.

FIG. 17000 illustrates an example of a method 1600.5 for monitoring a path of a person after determining their location relative to an object according to certain embodiments of this disclosure. The method 1600.5 may be performed by processing logic that may include hardware (circuitry, dedicated logic, etc.), software, or a combination of both. The method 1600.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000A) implementing the method 1600.5. The method 1600.5 may be implemented as computer instructions stored on a memory device and executable by the one or more processors. In certain implementations, the method 1600.5 may be performed by a single processing thread. Alternatively, the method 1600.5 may be performed by two or more processing threads, each thread implementing one or more individual functions, routines, subroutines, or operations of the methods.

At block 1602.5, the processing device may monitor, using data received from one or more smart floor tiles 112, a path of the person in the physical space. After the distance of the location of the person from the location of the object is determined, the smart floor tiles 112.5 may continuously or continually transmit obtained pressure measurements as the person walks around the physical space. The pressure measurements may include the identifiers of the smart floor tiles 112.5 and the times at which the smart floor tiles 112.5 obtain the pressure measurements, such that a path may be constructed by piecing together the pressure measurements from each smart floor tile 112.5 in a sequence over a time series.

Such a technique may enable determining if the person walks to another object where it is desirable to cause another action to be performed. For example, in a nursing home, a person having a neurodegenerative disease may walk near an exit door of the nursing home and the cloud-based computing system 116.5 may cause the door to lock. However, the person may continue to walk around the nursing home in search of another exit door. When the person is within a threshold distance of another door, as determined based on the data (e.g., pressure measurements) received from the smart floor tiles 112.5 near the another exit door, the cloud-based computing system 116.5 may transmit a control signal to the device to cause the device to perform an action. In this particular scenario, the control signal may include the path of the person.

At block 1604.5, the processing device may provide, to the device for presentation on a display screen of the device, the path of the person in the physical space. In some embodiments, the device may be the computing device 12.5 of the patient and/or the computing device 15.5 of a third-party (e.g., medical personnel, emergency responder, etc.). In some embodiments, a user interface displayed on the device may include graphical elements that enable the user to control other devices in the physical space. For example, the other devices may include actuation mechanisms, such as locks, alarms, electromechanical arms, and the like. The user may use the graphical elements to select to cause the actuation mechanisms to actuate. For example, in the scenario where a hostile person is present in the physical space, the user may cause the actuation mechanisms to close and/or lock, whether or not the hostile person is within a threshold distance to the objects (e.g., doors, windows) associated with the actuation mechanisms.

FIG. 18000 illustrates an example of a method for determining, based on data received from moulding section and smart floor tiles, a distance from a location of a person to a location of an object according to certain embodiments of this disclosure. The method 1700.5 may be performed by processing logic that may include hardware (circuitry, dedicated logic, etc.), software, or a combination of both. The method 1700.5 and/or each of their individual functions, subroutines, or operations may be performed by one or more processors of a computing device (e.g., any component (server 128.5, training engine 152.5, machine learning models 154.5, etc.) of cloud-based computing system 116.5 of FIG. 2000A) implementing the method 1700.5. The method 1700.5 may be implemented as computer instructions stored on a memory device and executable by the one or more processors. In certain implementations, the method 1700.5 may be performed by a single processing thread. Alternatively, the method 1700.5 may be performed by two or more processing threads, each thread implementing one or more individual functions, routines, subroutines, or operations of the methods.

At block 1702.5, the processing device may receive, from one or more moulding sections 102.5 located in the physical space, data pertaining to the location of the person. As previously discussed, the moulding sections may include one or more proximity sensors capable of detecting a presence of a person. In some embodiments, the proximity sensors may continuously or continually transmit the data pertaining to the presence of the person to the cloud-based computing system 116.5. In some embodiments, conserve power, the proximity sensors may be in a sleep mode when no movement is detected and transition to an active mode when movement is detected. Movement may be detected when an object (e.g., person) crosses a plane of a beam (e.g. laser, infrared, etc.) and/or vibration is detected as a person walks near the proximity sensor.

At block 1704.5, the processing device determine, based on the data received from the smart floor tiles 112.5 and the data 102.5 received from the moulding sections 102.5, the distance from the location of the person to the location of the object in the physical space. Using the data received from the moulding sections 102.5 and the data received from the smart floor tiles 112.5 may further increase the accuracy of determining a precise location of the person in the physical space than using one data source alone. Precisely determined location of the person may enable even more granular control of causing the device to perform an action (e.g., such that false positives pertaining to whether the person is within a threshold distance to the object may be avoided). As a result, processing resources may be saved because a control signal may not be transmitted in certain instances, thereby saving bandwidth of the network. If a control signal is not received by the device, the device may not perform an action, thereby saving processing resources of causing an actuation mechanism to actuate.

FIG. 19000 illustrates an example computer system 1800.5, which can perform any one or more of the methods described herein. In one example, computer system 1800.5 may include one or more components that correspond to the computing device 12.5, the computing device 15.5, one or more servers 128.5 of the cloud-based computing system 116.5, the electronic device 13.5, the camera 50.5, the moulding section 102.5, the smart floor tile 112.5, the device 1402.5, or one or more training engines 152.5 of the cloud-based computing system 116.5 of FIG. 2000A. The computer system 1800.5 may be connected (e.g., networked) to other computer systems in a LAN, an intranet, an extranet, or the Internet. The computer system 1800.5 may operate in the capacity of a server in a client-server network environment. The computer system 1800.5 may be a personal computer (PC), a tablet computer, a laptop, a wearable (e.g., wristband), a set-top box (STB), a personal Digital Assistant (PDA), a smartphone, a camera, a video camera, or any device capable of executing a set of instructions (sequential or otherwise) that specify actions to be taken by that device. Some or all of the components computer system 1800.5 may be included in the camera 50.5, the moulding section 102.5, and/or the smart floor tile 112.5. Further, while only a single computer system is illustrated, the term “computer” shall also be taken to include any collection of computers that individually or jointly execute a set (or multiple sets) of instructions to perform any one or more of the methods discussed herein.

The computer system 1800.5 includes a processing device 1802.5, a main memory 1804.5 (e.g., read-only memory (ROM), solid state drive (SSD), flash memory, dynamic random access memory (DRAM) such as synchronous DRAM (SDRAM)), a static memory 1806.5 (e.g., solid state drive (SSD), flash memory, static random access memory (SRAM)), and a data storage device 1808.5, which communicate with each other via a bus 1810.5.

Processing device 1802.5 represents one or more general-purpose processing devices such as a microprocessor, central processing unit, or the like. More particularly, the processing device 1802.5 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1802.5 may also be one or more special-purpose processing devices such as an application specific integrated circuit (ASIC), a field programmable gate array (FPGA), a digital signal processor (DSP), network processor, or the like. The processing device 1802.5 is configured to execute instructions for performing any of the operations and steps discussed herein.

The computer system 1800.5 may further include a network interface device 1812.5. The computer system 1800.5 also may include a video display 1814.5 (e.g., a liquid crystal display (LCD) or a cathode ray tube (CRT)), one or more input devices 1816.5 (e.g., a keyboard and/or a mouse), and one or more speakers 1818.5 (e.g., a speaker). In one illustrative example, the video display 1814.5 and the input device(s) 1816.5 may be combined into a single component or device (e.g., an LCD touch screen).

The data storage device 1816.5 may include a computer-readable medium 1820.5 on which the instructions 1822.5 embodying any one or more of the methodologies or functions described herein are stored. The instructions 1822.5 may also reside, completely or at least partially, within the main memory 1804.5 and/or within the processing device 1802.5 during execution thereof by the computer system 1800.5. As such, the main memory 1804.5 and the processing device 1802.5 also constitute computer-readable media. The instructions 1822.5 may further be transmitted or received over a network via the network interface device 1812.

While the computer-readable storage medium 1820.5 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The various aspects, embodiments, implementations or features of the described embodiments can be used separately or in any combination. The embodiments disclosed herein are modular in nature and can be used in conjunction with or coupled to other embodiments, including both statically-based and dynamically-based equipment. In addition, the embodiments disclosed herein can employ selected equipment such that they can identify individual users and auto-calibrate threshold multiple-of-body-weight targets, as well as other individualized parameters, for individual users.

Consistent with the above disclosure, the examples of systems and method enumerated in the following clauses are specifically contemplated and are intended as a non-limiting set of examples.

Clause 1. A method for correlating interaction effectiveness to contact time, the method comprising:

receiving, from a first set of one or more smart floor tiles, first data pertaining to one or more first time and location events caused by a first object in a first physical space, wherein the one or more first time and location events comprise one or more first times and one or more first locations of the first object in the first physical space;

receiving, from the first set of one or more smart floor tiles, second data pertaining to one or more second time and location events caused by a second object in the first physical space, wherein the one or more second time and location events comprise one or more second times and one or more second locations of the second object in the first physical space;

based on the first data and the second data, determining a first interaction time between the first object and the second object;

receiving first interaction effectiveness data pertaining to interaction effectiveness; and

generating a first time-effectiveness data point by associating the first interaction effectiveness data with the first interaction time.

Clause 2. The method of the preceding claim, further comprising:

receiving, from a second set of one or more smart floor tiles, third data pertaining to one or more third time and location events caused by a third object in a second physical space, wherein the one or more third time and location events comprise one or more third times and one or more third locations of the third object in the second physical space;

receiving, from the second set of one or more smart floor tiles, fourth data pertaining to one or more fourth time and location events caused by a fourth object in the second physical space, wherein the one or more fourth time and location events comprise one or more fourth times and one or more fourth locations of the fourth object in the second physical space;

based on the third data and the fourth data, determining a second interaction time between the third object and the fourth object;

receiving second interaction effectiveness data pertaining to interaction effectiveness; and

generating a second time-effectiveness data point by associating the second interaction effectiveness data with the second interaction time.

Clause 3. The method of any preceding clause, further comprising:

correlating the first time-effectiveness data point with the second time-effectiveness data point.

Clause 4. The method of any preceding clause, wherein the first object is a patient.

Clause 5. The method of any preceding clause, wherein the second object is a practitioner.

Clause 6. The method of any preceding clause, wherein the first object is a patient, the second object is a practitioner, and the first interaction time is a patient-to-practitioner contact time.

Clause 7. The method of any preceding clause, wherein the interaction effectiveness is a treatment effectiveness.

Clause 8. A system comprising:

a memory device storing instructions; and
a processing device communicatively coupled to the memory device, the processing device executes the instructions to:
receive, from a first set of one or more smart floor tiles, first data pertaining to one or more first time and location events caused by a first object in a first physical space, wherein the one or more first time and location events comprise one or more first times and one or more first locations of the first object in the first physical space;
receive, from the first set of one or more smart floor tiles, second data pertaining to one or more second time and location events caused by a second object in the first physical space, wherein the one or more second time and location events comprise one or more second times and one or more second locations of the second object in the first physical space;
based on the first data and the second data, determine a first interaction time between the first object and the second object;
receive first interaction effectiveness data pertaining to interaction effectiveness; and
generate a first time-effectiveness data point by associating the first interaction effectiveness data with the first interaction time.

Clause 9. The system of any preceding clause, wherein the instructions further cause the processing device to:

receive, from a second set of one or more smart floor tiles, third data pertaining to one or more third time and location events caused by a third object in a second physical space, wherein the one or more third time and location events comprise one or more third times and one or more third locations of the third object in the second physical space;

receive, from the second set of one or more smart floor tiles, fourth data pertaining to one or more fourth time and location events caused by a fourth object in the second physical space, wherein the one or more fourth time and location events comprise one or more fourth times and one or more fourth locations of the fourth object in the second physical space;

based on the third data and the fourth data, determine a second interaction time between the third object and the fourth object;

receive second interaction effectiveness data pertaining to interaction effectiveness; and

generate a second time-effectiveness data point by associating the second interaction effectiveness data with the second interaction time.

Clause 10. The system of any preceding clause, wherein the instructions further cause the processing device to:

correlate the first time-effectiveness data point with the second time-effectiveness data point.

Clause 11. The system of any preceding clause, wherein the first object is a patient.

Clause 12. The system of any preceding clause, wherein the second object is a practitioner.

Clause 13. The system of any preceding clause, wherein the first object is a patient, the second object is a practitioner, and the first interaction time is a patient-to-practitioner contact time.

Clause 14. The system of any preceding clause, wherein the interaction effectiveness is a treatment effectiveness.

Clause 15. A tangible, non-transitory computer-readable medium storing instructions that, when executed, cause a processing device to:

receive, from a first set of one or more smart floor tiles, first data pertaining to one or more first time and location events caused by a first object in a first physical space, wherein the one or more first time and location events comprise one or more first times and one or more first locations of the first object in the first physical space;

receive, from the first set of one or more smart floor tiles, second data pertaining to one or more second time and location events caused by a second object in the first physical space, wherein the one or more second time and location events comprise one or more second times and one or more second locations of the second object in the first physical space;

based on the first data and the second data, determine a first interaction time between the first object and the second object;

receive first interaction effectiveness data pertaining to interaction effectiveness; and

generate a first time-effectiveness data point by associating the first interaction effectiveness data with the first interaction time.

Clause 16. The tangible, non-transitory computer-readable medium of any preceding clause, wherein the instructions further cause the processing device to:

receive, from a second set of one or more smart floor tiles, third data pertaining to one or more third time and location events caused by a third object in a second physical space, wherein the one or more third time and location events comprise one or more third times and one or more third locations of the third object in the second physical space;

receive, from the second set of one or more smart floor tiles, fourth data pertaining to one or more fourth time and location events caused by a fourth object in the second physical space, wherein the one or more fourth time and location events comprise one or more fourth times and one or more fourth locations of the fourth object in the second physical space;

based on the third data and the fourth data, determine a second interaction time between the third object and the fourth object;

receive second interaction effectiveness data pertaining to interaction effectiveness; and

generate a second time-effectiveness data point by associating the second interaction effectiveness data with the second interaction time.

Clause 17. The tangible, non-transitory computer-readable medium of any preceding clause, wherein the instructions further cause the processing device to:

correlate the first time-effectiveness data point with the second time-effectiveness data point.

Clause 18. The tangible, non-transitory computer-readable medium of any preceding clause, wherein the first object is a patient.

Clause 19. The tangible, non-transitory computer-readable medium of any preceding clause, wherein the second object is a practitioner.

Clause 20. The tangible, non-transitory computer-readable medium of any preceding clause, wherein the first object is a patient, the second object is a practitioner, and the first interaction time is a patient-to-practitioner contact time.

Clause 21. The tangible, non-transitory computer-readable medium of any preceding clause, wherein the interaction effectiveness is a treatment effectiveness.

### Environment Control Using Moulding Sections

1.1 A method for environment control using a moulding section, the method comprising:

receiving data from a sensor in the moulding section;

determining, based on the data, whether a person is near the sensor;

determining an operating state of a device included in the moulding section, wherein the device performs the environment control of a physical space in which the moulding section is located; and

responsive to determining that the person is near the sensor and the operating state of the device, changing the device to operate in a second operating state to change a temperature of the physical space in which the moulding section is located.

2.1. The method of any preceding clause, further comprising:

receiving second data from a second sensor in the moulding section;

determining, based on the second data, the temperature of the environment in which the moulding section is located;

determining whether the temperature satisfies a threshold temperature condition; and

responsive to determining the temperature satisfies the threshold temperature condition, changing the operating state of the device to change the temperature of the physical space in which the moulding section is located.

3.1. The method of any preceding clause, further comprising:

receiving second data from the sensor in the moulding section;

determining, based on the second data, that the person is not near the sensor;

determining the second operating state of the device included in the moulding section; and

responsive to determining that the person is not near the sensor and the second operating state of the device, changing the device to operate in the operating state to change a temperature of the physical space in which the moulding section is located.

4.1. The method of any preceding clause, wherein the device is a fan.

5.1. The method of any preceding clause, wherein the sensor is a proximity sensor.

6.1. The method of any preceding clause, wherein the operating state is inactive and the second operating state is active.

7.1. The method of any preceding clause, further comprising:

receiving an instruction sent from a computing device external to the moulding section;

changing, based on the instruction, the device to operate in a third operating state to change the temperature of the physical space in which the moulding section is located.

8.1. The method of any preceding clause, further comprising:

determining whether the device is operating in the second operating state for a threshold period of time; and

responsive to determining the device is operating in the second operating state for the threshold period of time, changing the device to operate in the operating state.

9.1. A tangible, non-transitory computer-readable medium storing instructions that, when executed, cause a processing device to:

receive data from a sensor in a smart floor tile;

determine, based on the data, whether a person is present in a physical space including the smart floor tile;

determine an operating state of a device included in a moulding section, wherein the device performs environment control of the physical space in which the moulding section is located; and

responsive to determining that the person is present in the physical space and the operating state of the device, changing the device to operate in a second operating state to change a temperature of the physical space.

10.1 The computer-readable medium of any preceding clause, wherein the processing device is further to:

receive second data from a second sensor in the moulding section;

determine, based on the second data, the temperature of the environment in which the moulding section is located;

determine whether the temperature satisfies a threshold temperature condition; and

responsive to determining the temperature satisfies the threshold temperature condition, change the operating state of the device to change the temperature of the physical space in which the moulding section is located.

11.1 The computer-readable medium of any preceding clause, wherein the processing device is further to:

receive second data from the sensor;

determine, based on the second data, that the person is not present in the physical space;

determine the second operating state of the device included in the moulding section;

and

responsive to determining that the person is not present in the physical space and the second operating state of the device, change the device to operate in the operating state to change a temperature of the physical space in which the moulding section is located.

12.1. The computer-readable medium of any preceding clause, wherein the device is a fan.

13.1. The computer-readable medium of any preceding clause, wherein the sensor is a pressure sensor.

14.1. The computer-readable medium of any preceding clause, wherein the operating state is inactive and the second operating state is active.

15.1. The computer-readable medium of any preceding clause, wherein the processing device is further to:

receive an instruction sent from a computing device; and

change, based on the instruction, the device to operate in a third operating state to change the temperature of the physical space in which the moulding section is located.

16.1. The computer-readable medium of any preceding clause, wherein the processing device is further to:

determine whether the device is operating in the second operating state for a threshold period of time; and

responsive to determining the device is operating in the second operating state for the threshold period of time, change the device to operate in the operating state.

17.1. A moulding section comprising:

a memory device storing instructions;

a sensor;

an environment control device; and

a processing device communicatively coupled to the sensor, the environment control device, and the memory device, wherein the processing device executes the instructions to:

receive data from the sensor;

determine, based on the data, whether a person is near the sensor;

determine an operating state of the environment control device, wherein the environment control device performs environment control of a physical space in which the moulding section is located; and

responsive to determining that the person is near the sensor and the operating state of the environment control device, changing the environment control device to operate in a second operating state to change a temperature of the physical space in which the moulding section is located.

18.1. The moulding section of any preceding clause, wherein the processing device is further to:

receive second data from a second sensor in the moulding section;

determine, based on the second data, the temperature of the environment in which the moulding section is located;

determine whether the temperature satisfies a threshold temperature condition; and

responsive to determining the temperature satisfies the threshold temperature condition, change the operating state of the environment control device to change the temperature of the physical space in which the moulding section is located.

19.1. The moulding section of any preceding clause, wherein the processing device is further to:

receive second data from the sensor in the moulding section;

determine, based on the second data, that the person is not near the sensor;

determine the second operating state of the environment control device included in the moulding section; and

responsive to determining that the person is not near the sensor and the second operating state of the environment control device, change the environment control device to operate in the operating state to change a temperature of the physical space in which the moulding section is located.

20.1 The moulding section of any preceding clause, wherein the environment control device is a fan, the sensor is a proximity sensor, the operating state is inactive, and the second operating state is active.

### Security System Implemented in a Physical Space Using Smart Floor Tiles

1.2. A method for performing an action based on a location of a person in a physical space, the method comprising:

receiving, from one or more smart floor tiles located in the physical space, data pertaining to the location of the person, wherein the one or more smart floor tiles comprise one or more sensing devices capable of obtaining one or more pressure measurements, and the data comprises the one or more pressure measurements;

determining, based on the data, a distance from the location of the person to a location of an object in the physical space;

determining whether the distance from the location of the person to the location of the object satisfies a threshold distance; and

responsive to determining the distance satisfies the threshold distance, transmitting, via a processing device, a control signal to a device to cause the device to perform an action, wherein the device is distal from the processing device.

2.2. The method of any preceding clause, further comprising, prior to determining the distance from the location of the person to the location of the object:

determining an identity of the person based on second data, wherein the second data comprises:

an identifier of the physical space in which the one or more smart floor tiles are located,

an identifier of the person associated with the identifier of the physical space,

a weight of the person determined based on the one or more pressure measurements,

a time of day the data is received,

an image of the person obtained via a camera in the physical space,

a stored image of the person, or

some combination thereof; and

determining whether the identity of the person is included in a list.

3.2. The method of any preceding clause, wherein the object is an ingress or egress to the physical space.

4.2. The method of any preceding clause, wherein the object is a door or a window, the device comprises an actuation mechanism, and the action comprises causing the actuation mechanism to actuate.

5.2. The method of any preceding clause, wherein causing the actuation mechanism to actuate further comprises:

actuating the actuation mechanism to lock or unlock,

actuating the actuation mechanism to open or close the door or the window, or

some combination thereof.

6.2. The method of any preceding clause wherein the action comprises:

(i) actuating to open the object, to close the object, to lock the object, to unlock the object, or some combination thereof,

(ii) presenting, on a display screen of the device, a notification including information pertaining to the person, the location of the person, the distance satisfying the threshold distance, or some combination thereof,

(iii) triggering an alarm,

(iv) enabling, via a speaker of the device, communicating with the person in the physical space,

(v) dispatching an emergency service, or

(v) some combination thereof.

7.2. The method of any preceding clause, further comprising:

monitoring, using subsequent data received from the one or more smart floor tiles, a path of the person in the physical space; and

providing, to the device for presentation on a display screen of the device, the path of the person in the physical space.

8.2. The method of any preceding clause, further comprising:

receiving, from one or more moulding sections located in the physical space, second data pertaining to the location of the person, wherein the one or more moulding sections comprise one or more proximity sensors capable of obtaining the second data pertaining to the location of the person; and

determining, based on the data and the second data, the distance from the location of the person to the location of the object in the physical space.

9.2. The method of any preceding clause, wherein the physical space is a nursing home, a hospital, a school, a movie theater, a theater, a stadium, an office, a house, an airport, a bus station, a train station, a port, an auditorium, a cafeteria, a restaurant, a building, a park, a parking garage, or some combination thereof.

10.2. A tangible, non-transitory computer-readable medium storing instructions that, when executed, cause a processing device to:

receive, from one or more smart floor tiles located in a physical space, data pertaining to a location of a person, wherein the one or more smart floor tiles comprise one or more sensing devices capable of obtaining one or more pressure measurements, and the data comprises the one or more pressure measurements;

determine, based on the data, a distance from the location of the person to a location of an object in the physical space;

determine whether the distance from the location of the person to the location of the object satisfies a threshold distance; and

responsive to determining the distance satisfies the threshold distance, transmit, via a processing device, a control signal to a device to cause the device to perform an action, wherein the device is distal from the processing device.

11.2. The computer-readable medium of any preceding clause, wherein the processing device is further to, prior to determining the distance from the location of the person to the location of the object:

determine an identity of the person based on second data, wherein the second data comprises:

an identifier of the physical space in which the one or more smart floor tiles are located,

an identifier of the person associated with the identifier of the physical space,

a weight of the person determined based on the one or more pressure measurements,

a time of day the data is received,

an image of the person obtained via a camera in the physical space,

a stored image of the person, or

some combination thereof; and

determine whether the identity of the person is included in a list.

12.2. The computer-readable medium of any preceding clause, wherein the object is an ingress or egress to the physical space.

13.2. The computer-readable medium of any preceding clause, wherein the object is a door or a window, the device comprises an actuation mechanism, and the action comprises causing the actuation mechanism to actuate.

14.2 The computer-readable medium of any preceding clause, wherein causing the actuation mechanism to actuate further comprises:

actuating the actuation mechanism to lock or unlock,

actuating the actuation mechanism to open or close the door or the window, or

some combination thereof.

15.2. The computer-readable medium of any preceding clause, wherein the action comprises:

(i) actuating to open the object, to close the object, to lock the object, to unlock the object, or some combination thereof,

(ii) presenting, on a display screen of the device, a notification including information pertaining to the person, the location of the person, the distance satisfying the threshold distance, or some combination thereof,

(iii) triggering an alarm,

(iv) enabling, via a speaker of the device, communicating with the person in the physical space,

(v) dispatching an emergency service, or

(v) some combination thereof.

16.2. The computer-readable medium of any preceding clause, wherein the processing device is further to:

monitor, using subsequent data received from the one or more smart floor tiles, a path of the person in the physical space; and

provide, to the device for presentation on a display screen of the device, the path of the person in the physical space.

17.2. The computer-readable medium of any preceding clause, wherein the processing device is further to:

receive, from one or more moulding sections located in the physical space, second data pertaining to the location of the person, wherein the one or more moulding sections comprise one or more proximity sensors capable of obtaining the second data pertaining to the location of the person; and

determine, based on the data and the second data, the distance from the location of the person to the location of the object in the physical space.

18.2. The computer-readable medium of any preceding clause, wherein the physical space is a nursing home, a hospital, a school, a movie theater, a theater, a stadium, an office, a house, an airport, a bus station, a train station, a port, an auditorium, a cafeteria, a restaurant, a building, a park, a parking garage, or some combination thereof.

19.2. A system comprising:

a memory device storing instructions;

a processing device communicatively coupled to the memory device, the processing device executes the instructions to:

receive, from one or more smart floor tiles located in a physical space, data pertaining to a location of a person, wherein the one or more smart floor tiles comprise one or more sensing devices capable of obtaining one or more pressure measurements, and the data comprises the one or more pressure measurements;

determine, based on the data, a distance from the location of the person to a location of an object in the physical space;

determine whether the distance from the location of the person to the location of the object satisfies a threshold distance; and

responsive to determining the distance satisfies the threshold distance, transmit, via a processing device, a control signal to a device to cause the device to perform an action, wherein the device is distal from the processing device.

20.2 The system of any preceding clause, wherein the processing device is further to, prior to determining the distance from the location of the person to the location of the object:

determine an identity of the person based on second data, wherein the second data comprises:

an identifier of the physical space in which the one or more smart floor tiles are located,

an identifier of the person associated with the identifier of the physical space,

a weight of the person determined based on the one or more pressure measurements,

a time of day the data is received,

an image of the person obtained via a camera in the physical space,

a stored image of the person, or

some combination thereof; and

determine whether the identity of the person is included in a list.

The various aspects, embodiments, implementations or features of the described embodiments can be used separately or in any combination. The embodiments disclosed herein are modular in nature and can be used in conjunction with or coupled to other embodiments, including both statically-based and dynamically-based equipment. In addition, the embodiments disclosed herein can employ selected equipment such that they can identify individual users and auto-calibrate threshold multiple-of-body-weight targets, as well as other individualized parameters, for individual users.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

1. A method for correlating interaction effectiveness to contact time, the method comprising:
receiving, from a first set of one or more smart floor tiles, first data pertaining to one or more first time and location events caused by a first object in a first physical space, wherein the one or more first time and location events comprise one or more first times and one or more first locations of the first object in the first physical space;
receiving, from the first set of one or more smart floor tiles, second data pertaining to one or more second time and location events caused by a second object in the first physical space, wherein the one or more second time and location events comprise one or more second times and one or more second locations of the second object in the first physical space;
based on the first data and the second data, determining a first interaction time between the first object and the second object;
receiving first interaction effectiveness data pertaining to interaction effectiveness; and
generating a first time-effectiveness data point by associating the first interaction effectiveness data with the first interaction time.

2. The method of claim 1, further comprising:
receiving, from a second set of one or more smart floor tiles, third data pertaining to one or more third time and location events caused by a third object in a second physical space, wherein the one or more third time and location events comprise one or more third times and one or more third locations of the third object in the second physical space;
receiving, from the second set of one or more smart floor tiles, fourth data pertaining to one or more fourth time and location events caused by a fourth object in the second physical space, wherein the one or more fourth time and location events comprise one or more fourth times and one or more fourth locations of the fourth object in the second physical space;
based on the third data and the fourth data, determining a second interaction time between the third object and the fourth object;
receiving second interaction effectiveness data pertaining to interaction effectiveness; and
generating a second time-effectiveness data point by associating the second interaction effectiveness data with the second interaction time.

3. The method of claim 2, further comprising:
correlating the first time-effectiveness data point with the second time-effectiveness data point.

4. The method of claim 1, wherein the first object is a patient.

5. The method of claim 1, wherein the second object is a practitioner.

6. The method of claim 1, wherein the first object is a patient, the second object is a practitioner, and the first interaction time is a patient-to-practitioner contact time.

7. The method of claim 1, wherein the interaction effectiveness is a treatment effectiveness.

8. A system comprising:
a memory device storing instructions; and
a processing device communicatively coupled to the memory device, the processing device executes the instructions to:
receive, from a first set of one or more smart floor tiles, first data pertaining to one or more first time and location events caused by a first object in a first physical space, wherein the one or more first time and location events comprise one or more first times and one or more first locations of the first object in the first physical space;
receive, from the first set of one or more smart floor tiles, second data pertaining to one or more second time and location events caused by a second object in the first physical space, wherein the one or more second time and location events comprise one or more second times and one or more second locations of the second object in the first physical space;
based on the first data and the second data, determine a first interaction time between the first object and the second object;
receive first interaction effectiveness data pertaining to interaction effectiveness; and
generate a first time-effectiveness data point by associating the first interaction effectiveness data with the first interaction time.

9. The system of claim 8, wherein the instructions further cause the processing device to:
receive, from a second set of one or more smart floor tiles, third data pertaining to one or more third time and location events caused by a third object in a second physical space, wherein the one or more third time and location events comprise one or more third times and one or more third locations of the third object in the second physical space;
receive, from the second set of one or more smart floor tiles, fourth data pertaining to one or more fourth time and location events caused by a fourth object in the second physical space, wherein the one or more fourth time and location events comprise one or more fourth times and one or more fourth locations of the fourth object in the second physical space;
based on the third data and the fourth data, determine a second interaction time between the third object and the fourth object;
receive second interaction effectiveness data pertaining to interaction effectiveness; and
generate a second time-effectiveness data point by associating the second interaction effectiveness data with the second interaction time.

10. The system of claim 9, wherein the instructions further cause the processing device to:
correlate the first time-effectiveness data point with the second time-effectiveness data point.

11. The system of claim 8, wherein the first object is a patient.

12. The system of claim 8, wherein the second object is a practitioner.

13. The system of claim 8, wherein the first object is a patient, the second object is a practitioner, and the first interaction time is a patient-to-practitioner contact time.

14. The system of claim 8, wherein the interaction effectiveness is a treatment effectiveness.

15. A tangible, non-transitory computer-readable medium storing instructions that, when executed, cause a processing device to:
receive, from a first set of one or more smart floor tiles, first data pertaining to one or more first time and location events caused by a first object in a first physical space, wherein the one or more first time and location events comprise one or more first times and one or more first locations of the first object in the first physical space;
receive, from the first set of one or more smart floor tiles, second data pertaining to one or more second time and location events caused by a second object in the first physical space, wherein the one or more second time and location events comprise one or more second times and one or more second locations of the second object in the first physical space;
based on the first data and the second data, determine a first interaction time between the first object and the second object;
receive first interaction effectiveness data pertaining to interaction effectiveness; and
generate a first time-effectiveness data point by associating the first interaction effectiveness data with the first interaction time.

16. The tangible, non-transitory computer-readable medium of claim 15, wherein the instructions further cause the processing device to:
receive, from a second set of one or more smart floor tiles, third data pertaining to one or more third time and location events caused by a third object in a second physical space, wherein the one or more third time and location events comprise one or more third times and one or more third locations of the third object in the second physical space;
receive, from the second set of one or more smart floor tiles, fourth data pertaining to one or more fourth time and location events caused by a fourth object in the second physical space, wherein the one or more fourth time and location events comprise one or more fourth times and one or more fourth locations of the fourth object in the second physical space;
based on the third data and the fourth data, determine a second interaction time between the third object and the fourth object;
receive second interaction effectiveness data pertaining to interaction effectiveness; and
generate a second time-effectiveness data point by associating the second interaction effectiveness data with the second interaction time.

17. The tangible, non-transitory computer-readable medium of claim 16, wherein the instructions further cause the processing device to:
correlate the first time-effectiveness data point with the second time-effectiveness data point.

18. The tangible, non-transitory computer-readable medium of claim 15, wherein the first object is a patient.

19. The tangible, non-transitory computer-readable medium of claim 15, wherein the second object is a practitioner.

20. The tangible, non-transitory computer-readable medium of claim 15, wherein the first object is a patient, the second object is a practitioner, and the first interaction time is a patient-to-practitioner contact time.
